(19) United States
(12) Patent Application Publication
(10) Pub. No.: US 2008/0255252 A1
(43) Pub. Date: Oct. 16, 2008

(54) Infectious Aetiology of Prostatic Disease and Methods to Identify Causative Agents

(75) Inventors: Ronald Joseph Cohen, Dianella (AU); Kerryn Lee Garrett, Shenton Park (AU); Beverly Anne Shannon, Dianella (AU)

Correspondence Address: MORRISON & FOERSTER LLP, 755 PAGE MILL RD, PALO ALTO, CA, 94304-1018, US

(73) Assignee: Tissugen Pty. Ltd., Western Austalia (AU)

(21) Appl. No.: 10/592,579

(22) PCT Filed: Mar. 15, 2005
(86) PCT No.: PCT/AU05/00355

§ 371 Date: Jan. 28, 2008

Related U.S. Application Data
(60) Provisional application No. 60/612,688, filed on Sep. 24, 2004.

(30) Foreign Application Priority Data
Mar. 15, 2004 (AU) 2004901359

(51) Int. Cl. A61K 45/00; C12Q 1/68; C12N 15/31; C12N 1/20; A61P 31/04; A61P 13/08

(52) U.S. Cl. 514/789; 536/23.7; 536/24.33; 536/24.32; 435/6; 435/252.1

(57) ABSTRACT

The present invention relates to a method of diagnosing, or predicting risk of, prostate disease in a subject. More particularly, the invention relates to a method of diagnosing the presence of, or the predisposition to develop, prostate disease in a subject, the method comprising analysing a test sample from the subject for the presence of P. acnes infection of the prostate gland. The present invention further relates to reagents for use in this method and to methods of prevention or treatment of prostate disease.

### TECHNICAL FIELD

The present invention relates to a method of diagnosing, or predicting risk of, prostate disease in a subject. The present invention further relates to reagents for use in this method and to methods of prevention or treatment of prostate disease.

### BACKGROUND OF THE INVENTION

Diseases of the prostate include prostate cancer, which is the most common form of cancer occurring in males. Therefore, methods for detecting a predisposition or relative risk of cancer developing are of clinical importance to allow preventative treatment regimes to be implemented. Such methods could be based upon detection of various disease markers in lest samples such as blood, serum, plasma, urine or prostatic secretions. Procedures would provide a reduction in the development of prostate cancer in many men.

Prostatic inflammation and its subsequent sequelae, post-inflammatory atrophy, is a pathological condition seen in most men with prostate disease. Chronic inflammation, as manifest by infiltration of lymphocytes and macrophages into the prostatic stroma surrounding glands, is reported in 98-100% of cases (Kohnen 1979, McClinton 1990, Blumenfeld 1992, Irani 1999, De Marzo 1999, Gerstenbluth 2002). Acute (active) inflammation involving infiltration of polymorphonuclear leukocytes into the glandular epithelium, often with associated intraluminal microabscesses, is described in 20-85% of cases (Kohnen 1979, Irani 1999, De Marzo 1999). This inflammation cannot be explained simply as an immune response to carcinoma as it is also commonly found in prostate glands devoid of malignancy (Kohnen 1979, McClinton 1990, Irani 1997). Furthermore, even in prostate specimens involved by tumour, inflammation is focal and scattered throughout the entire organ rather than confined to the tumour or its periphery (Blumenfeld 1992, Irani 1999). The targeting of various factors of the inflammatory process for chemoprevention of prostate cancer has also been proposed (Lucia and Torkko 2004).

The aetiology of prostatic inflammation is currently unknown, however similar findings of inflammation, atrophy, dysplasia and malignancy are seen in other organ systems including the association of atrophic gastritis, gastric dysplasia, carcinoma and Helicobacter pylori infection (Ucmura 2001, Faraji 2002). Similarly, the possibility of an infectious origin for prostate cancer has been suggested (Dudgeon 1904, Rosen 1918, Keay 1999, Krieger 2000, Dennis 2001, Strickler 2001). Focal post-inflammatory glandular atrophy was proposed as a possible precursor of prostate carcinoma by Franks in 1954, while later studies (Smith 1987, Platz 1998) implicated chronic prostatic inflammation in the development of malignancy. Recent work (De Marzo, 1999; Platz and De Marzo 2004) integrates these theories, suggesting that prostate carcinoma originates from lesions of proliferative inflammatory atrophy, which are frequently seen to merge with regions of high-grade dysplasia (prostatic intra-epithelial neoplasia); a widely accepted precursor lesion for prostate carcinoma (McNeal 1969).

There also exists epidemiological evidence linking prostate cancer to infectious agents such as those associated with sexually transmitted diseases particularly among African-Americans, a racial group with one of the highest rates of prostate cancer (Dennis 2001, Strickler 2001). A second link is suggested by the recent finding that mutations in two genes involved in the immune response to infectious agents are associated with development of prostate cancer (reviewed in DeMarzo 2003). One particular variation of the RNasel gene, which mediates apoptosis of virally infected cells, has been implicated in development of up to 13% of unselected prostate cancer cases (Silverman 2003). Further, mutations of the MSRI gene, coding for a macrophage receptor involved in phagocytosis of bacterial pathogens, have been associated with increased risk of prostate cancer, particularly in African-American men (Xu 2002). However, the role of bacterial agents in diseases of the prostate has been poorly investigated.

Propionibacterium acnes is a ubiquitous organism that is microaerophilic and found predominantly in skin appendages (sweat and sebaceous glands) where it represents a commensal organism. However, this organism is responsible for the metabolism of lipids and production of irritant compounds, which are thought to be responsible for androgen-induced acne vulgaris (Webster 1995). Further, inoculation of this organism into wounds can result in chronic infections that may persist for decades (Sabel 1999; Brook 1991). Recently this organism has been identified in tissues involved in chronic inflammatory diseases such as sarcoidosis (Yamada 2002) and sciatica (Stirling 2001). Genomic DNA sequences of skin associated P. acnes have been analyzed and described in WO 01/81581 and WO 03/033515.

The present inventors have consistently identified Propionibacterium acnes in prostate tissue removed as part of the treatment for prostate cancer. The identification of P. acnes correlated strongly with the presence of inflammation, both acute and chronic, observed microscopically within the prostate gland. Therefore, the presence of P. acnes in prostate tissue represents a potential aetiological and/or risk factor in the development of prostate cancer due to its ability to initiate an inflammatory process.

### SUMMARY OF THE INVENTION

The prostate gland is typically believed to be sterile and has no normal flora. The present inventors have, however, consistently isolated and cultured Propionibacterium acnes from prostate tissue of patients with prostate disease. Further, the present inventors have determined that the P. acnes typically isolated from diseased prostate tissue differ from the common skin isolates of P. acnes. The present inventors identified that P. acnes, more commonly Group 2 and/or Group 3 defined herein, are associated with prostate diseases (such as prostatitis, dysplasia, and prostate cancer), and that P. acnes, more commonly Group 2 and/or Group 3 are present in the prostate tissue of patients having prostate diseases.

Accordingly, in a first aspect of the present invention there is provided a method of diagnosing the presence of, or the predisposition to develop, prostate disease in a subject, the method comprising analysing a lest sample from the subject for the presence of P. acnes infection of the prostate gland.

In a second aspect of the present invention there is provided an isolated polynucleotide, wherein the isolated polynucleotide has a sequence selected from the group consisting of: SEQ ID NO:1;

a sequence at least 99% identical to SEQ ID NO:1;
a fragment of at least about 10, at least about 15, at least about 20, at least about 25, at least about 30, at least about 50, at least about 100, at least about 150, at least about 200 or more contiguous nucleotides of SEQ ID NO:1, wherein the sequence comprises a sequence that is not present in P. acnes Group 1 or that is specific for P. acnes Group 3; a sequence which hybridizes to SEQ ID NO:1 under conditions of high stringency;
a sequence complementary to SEQ ID NO:1 or a fragment of SEQ ID NO:1 described above. SEQ ID NO:1;

### SEQ ID NO:2;

a sequence at least 99% identical to SEQ ID NO:2;
a fragment of at least about 10, at least about 15, at least about 20, at least about 25, at least about 30, at least about 50, at least about 100, at least about 150, at least about 200or more contiguous nucleotides of SEQ ID NO:2, wherein the sequence comprises a sequence that is not present in P. acnes Group 3 or that is specific for P. acnes Group 1 or 2;
a sequence which hybridizes to SEQ ID NO:2 under conditions of high stringency;
a sequence complementary to SEQ ID NO:2 or a fragment of SEQ ID NO:2 described above,

### SEQ ID NO:6;

a sequence at least 95%, preferably at least 96%, more preferably at least 97%, even more preferably at least 98%, and more preferably at least 99% identical to SEQ ID NO:6;
a fragment of at least about 10, at least about 15, at least about 20, at least about 25, at least about 30, at least about 50, at least about 100, at least about 150, at least about 200or more contiguous nucleotides of SEQ ID NO:6, wherein the sequence comprises a sequence that is not present in P. acnes Group 1 or that is specific for P. acnes Group 2 and/or Group 3;
a sequence which hybridizes to SEQ ID NO:6 under conditions of high stringency;
a sequence complementary to SEQ ID NO:6 or a fragment of SEQ ID NO:6 described above;

### SEQ ID NO:11;

a sequence at least 95%, preferably at least 96%, more preferably at least 97%, even more preferably at least 98%, and more preferably at least 99% identical to SEQ ID NO:11;
a fragment of at least about 10, at least about 15, at least about 20, at least about 25, at least about 30, at least about 50, at least about 100, at least about 150, at least about 200or more contiguous nucleotides of SEQ ID NO:11, wherein the fragment comprises a sequence that is not present in P. acnes Group 1 or that is specific for P. acnes Group 2 and/or Group 3;
a sequence which hybridizes to SEQ ID NO:11 under conditions of high stringency;
a sequence complementary to SEQ ID NO:11 or a fragment of SEQ ID NO:11 described above;

### SEQ ID NO:15;

a sequence at least 95%, preferably at least 96%, more preferably at least 97%, even more preferably at least 98%, and more preferably at least 99% identical to SEQ ID NO:15;
a fragment of at least 10, at least 15, at least 20, at least 25, at least 30, at least 50, at least 100, at least 150, at least 200 or more contiguous nucleotides of SEQ ID NO:15, wherein the sequence comprises a sequence that is not present in P. acnes Group 2 and/or Group 3or that is specific for P. acnes Group 1;
a sequence which hybridizes to SEQ ID NO:15 under conditions of high stringency;
a sequence complementary to SEQ ID NO:15 or a fragment of SEQ ID NO:15 described above;

### SEQ ID NO:16;

a sequence at least 95%, preferably at least 96%, more preferably at least 97%, even more preferably at least 98%, and more preferably at least 99% identical to SEQ ID NO:16;
a fragment of at least 10, at least 15, at least 20, at least 25, at least 30, at least 50, at least 100, at least 150, at least 200 or more contiguous nucleotides of SEQ ID NO:16, wherein the sequence comprises a sequence that is not present in P. acnes Group 1 and/or Group 3or that is specific for P. acnes Group 2;
a sequence which hybridizes to SEQ ID NO:16 under conditions of high stringency;
a sequence complementary to SEQ ID NO:16 or a fragment of SEQ ID NO:16 described above;

### SEQ ID NO:17;

a sequence at least 95%, preferably at least 96%, more preferably at least 97%, even more preferably at least 98%, and more preferably at least 99% identical to SEQ ID NO:17;
a fragment of at least 10, at least 15, at least 20, at least 25, at least 30, at least 50, at least 100, at least 150, at least 200 or more contiguous nucleotides of SEQ ID NO:17, wherein the sequence comprises a sequence that is not present in P. acnes Group 1 and/or Group 2 or that is specific for P. acnes Group 3;
a sequence which hybridizes to SEQ ID NO:17 under conditions of high stringency;
a sequence complementary to SEQ ID NO:17 or a fragment of SEQ ID NO:17 described above;

### SEQ ID NO:20;

a sequence at least 95%, preferably at least 96%, more preferably at least 97%, even more preferably at least 98%, and more preferably at least 99% identical to SEQ ID NO:20;
a fragment of at least 10, at least 15, at least 20, at least 25, at least 30, at least 50, at least 100, at least 150, at least 200 or more contiguous nucleotides of SEQ ID NO:20, wherein the sequence comprises a sequence that is not present in P. acnes Group 2 and/or Group 3or that is specific for P. acnes Group 1;
a sequence which hybridizes to SEQ ID NO:20 under conditions of high stringency;
a sequence complementary to SEQ ID NO:20 or a fragment of SEQ ID NO:20 described above;

### SEQ ID NO:23;

a sequence at least 95%, preferably at least 96%, more preferably at least 97%, even more preferably at least 98%, and more preferably at least 99% identical to SEQ ID NO:23;
a fragment of at least 10, at least 15, at least 20, at least 25, at least 30, at least 50, at least 100, at least 150, at least 200 or more contiguous nucleotides of SEQ ID NO:23, wherein the sequence comprises a sequence that is not present in P. acnes Group 2 and/or Group 3or that is specific for P. acnes Group 1;
a sequence which hybridizes to SEQ ID NO:23 under conditions of high stringency;
a sequence complementary to SEQ ID NO:23 or a fragment of SEQ ID NO:23 described above;

### SEQ ID NO:26;

a sequence at least 95%, preferably at least 96%, more preferably at least 97%, even more preferably at least 98%, and more preferably at least 99% identical to SEQ ID NO:26;
a fragment of at least 10, at least 15, at least 20, at least 25, at least 30, at least 50, at least 100, at least 150, at least 200 or more contiguous nucleotides of SEQ ID NO:26, wherein the sequence comprises a sequence that is not present in P. acnes Group 2 and/or Group 3or that is specific for P. acnes Group 1;
a sequence which hybridizes to SEQ ID NO:26 under conditions of high stringency;
a sequence complementary to SEQ ID NO:23 or a fragment of SEQ ID NO:23 described above;

### SEQ ID NO:27;

a sequence at least 95%, preferably at least 96%, more preferably at least 97%, even more preferably at least 98%, and more preferably at least 99% identical to SEQ ID NO:27;
a fragment of at least 10, at least 15, at least 20, at least 25, at least 30, at least 50, at least 100, at least 150, at least 200 or more contiguous nucleotides of SEQ ID NO:27, wherein the sequence comprises a sequence that is not present in P. acnes Group 1 and/or Group 3or that is specific for P. acnes Group 2;
a sequence which hybridizes to SEQ ID NO:27 under conditions of high stringency;
a sequence complementary to SEQ ID NO:27 or a fragment of SEQ ID NO:27 described above;

### SEQ ID NO:28;

a sequence at least 95%, preferably at least 96%, more preferably at least 97%, even more preferably at least 98%, and more preferably at least 99% identical to SEQ ID NO:28;
a fragment of at least 10, at least 15, at least 20, at least 25, at least 30, at least 50, at least 100, at least 150, at least 200 or more contiguous nucleotides of SEQ ID NO:28, wherein the sequence comprises a sequence that is not present in P. acnes Group 1 and/or Group 2 or that is specific for P. acnes Group 3;
a sequence which hybridizes to SEQ ID NO:28 under conditions of high stringency;
a sequence complementary to SEQ ID NO:28 or a fragment of SEQ ID NO:28 described above;

### SEQ ID NO:31;

a sequence at least 95%, preferably at least 96%, more preferably at least 97%, even more preferably at least 98%, and more preferably at least 99% identical to SEQ ID NO:31;
a fragment of at least 10, at least 15, at least 20, at least 25, at least 30, at least 50, at least 100, at least 150, at least 200 or more contiguous nucleotides of SEQ ID NO:31, wherein the sequence comprises a sequence that is not present in P. acnes Group 2 and/or Group 3or that is specific for P. acnes Group 1;
a sequence which hybridizes to SEQ ID NO:31 under conditions of high stringency;
a sequence complementary to SEQ ID NO:31 or a fragment of SEQ ID NO:31 described above;

### SEQ ID NO:32;

a sequence at least 95%, preferably at least 96%, more preferably at least 97%, even more preferably at least 98%, and more preferably at least 99% identical to SEQ ID NO:32;
a fragment of at least 10, at least 15, at least 20, at least 25, at least 30, at least 50, at least 100, at least 150, at least 200 or more contiguous nucleotides of SEQ ID NO:32, wherein the sequence comprises a sequence that is not present in P. acnes Group 1 and/or Group 3or that is specific for P. acnes Group 2;
a sequence which hybridizes to SEQ ID NO:32 under conditions of high stringency;
a sequence complementary to SEQ ID NO:32 or a fragment of SEQ ID NO:32 described above;

### SEQ ID NO:35;

a sequence at least 95%, preferably at least 96%, more preferably at least 97%, even more preferably at least 98%, and more preferably at least 99% identical to SEQ ID NO:35;
a fragment of at least 10, at least 15, at least 20, at least 25, at least 30, at least 50, at least 100, at least 150, at least 200 or more contiguous nucleotides of SEQ ID NO:35, wherein the sequence comprises a sequence that is not present in P. acnes Group 2 and/or Group 3or that is specific for P. acnes Group 1;
a sequence which hybridizes to SEQ ID NO:35 under conditions of high stringency;
a sequence complementary to SEQ ID NO:35 or a fragment of SEQ ID NO:35 described above;

### SEQ ID NO:36;

a sequence at least 95%, preferably at least 96%, more preferably at least 97%, even more preferably at least 98%, and more preferably at least 99% identical to SEQ ID NO:36;
a fragment of at least 10, at least 15, at least 20, at least 25, at least 30, at least 50, at least 100, at least 150, at least 200 or more contiguous nucleotides of SEQ ID NO:36, wherein the sequence comprises a sequence that is not present in P. acnes Group 1 and/or Group 3or that is specific for P. acnes Group 2;
a sequence which, hybridises to SEQ ID NO:36 under conditions of high stringency;
a sequence complementary to SEQ ID NO:36 or a fragment of SEQ ID NO:36 described above;

### SEQ ID NO:39;

a sequence at least 95%, preferably at least 96%, more preferably at least 97%, even more preferably at least 98%, and more preferably at least 99% identical to SEQ ID NO:39;
a fragment of at least 10, at least 15, at least 20, at least 25, at least 30, at least 50, at least 100, at least 150, at least 200 or more contiguous nucleotides of SEQ ID NO:39, wherein the sequence comprises a sequence that is not present in P. acnes Group 1 and/or Group 3or that is specific for P. acnes Group 2;
a sequence which hybridizes to SEQ ID NO:39 under conditions of high stringency;
a sequence complementary to SEQ ID NO:39 or a fragment of SEQ ID NO:39 described above;

### SEQ ID NO:42;

a sequence at least 95%, preferably at least 96%, more preferably at least 97%, even more preferably at least 98%, and more preferably at least 99% identical to SEQ ID NO:42;
a fragment of at least 10, at least 15, at least 20, at least 25, at least 30, at least 50, at least 100, at least 150, at least 200 or more contiguous nucleotides of SEQ ID NO:42, wherein the sequence comprises a sequence that is not present in P. acnes Group 1 and/or Group 3or that is specific for P. acnes Group 2;
a sequence which hybridizes to SEQ ID NO:42 under conditions of high stringency;
a sequence complementary to SEQ ID NO:42 or a fragment of SEQ ID NO:42 described above;

### SEQ ID NO:45;

a sequence at least 95%, preferably at least 96%, more preferably at least 97%, even more preferably at least 98%, and more preferably at least 99% identical to SEQ ID NO:45;
a fragment of at least 10, at least 15, at least 20, at least 25, at least 30, at least 50, at least 100, at least 150, at least 200 or more contiguous nucleotides of SEQ ID NO:45, wherein the sequence comprises a sequence that is not present in P. acnes Group 3 that is specific for P. acnes Group 1 or Group 2;
a sequence which hybridizes to SEQ ID NO:45 under conditions of high stringency;
a sequence complementary to SEQ ID NO:45 or a fragment of SEQ ID NO:45 described above;

### SEQ ID NO:46;

a sequence at least 95%, preferably at least 96%, more preferably at least 97%, even more preferably at least 98%, and more preferably at least 99% identical to SEQ ID NO:46;
a fragment of at least 10, at least 15, at least 20, at least 25, at least 30, at least 50, at least 100, at least 150, at least 200 or more contiguous nucleotides of SEQ ID NO:46, wherein the sequence comprises a sequence that is not present in P. acnes Group 1 or Group 2 that is specific for P. acnes Group 3;
a sequence which hybridizes to SEQ ID NO:46 under conditions of high stringency;
a sequence complementary to SEQ ID NO:46 or a fragment of SEQ ID NO:46 described above.

In a third aspect, the present invention provides a primer wherein the primer binds specifically to a polynucleotide according to the second aspect of die invention.

In a fourth aspect, the present invention provides a primer sequence that distinguishes between Group 1, Group 2 and Group 3 P. acnes as herein defined.

In a preferred embodiment the primer sequence according to the fourth aspect specifically binds to SEQ ID NO. 1, SEQ ID NO. 2, SEQ ID NO. 6, SEQ ID NO. 11, SEQ ID NO. 15, SEQ ID NO. 16, SEQ ID NO. 17, SEQ ID NO. 20, SEQ ID NO. 23, SEQ ID NO. 26, SEQ ID NO. 27, SEQ ID NO. 28, SEQ ID NO. 31, SEQ ID NO. 32, SEQ ID NO. 35, SEQ ID NO. 36, SEQ ID NO. 39, SEQ ID NO. 42, SEQ ID NO. 45 and SEQ ID NO. 46.

In a fifth aspect, the present invention provides a probe specific for P. acnes, wherein the probe detects or or localizes a P. acnes nucleic acid sequence or antigen.

In a sixth aspect the present invention provides a kit for diagnosing the presence of, or the predisposition to develop, prostate disease in a subject, the kit comprising at least one P. acnes specific probe.

In a seventh aspect, the present invention also provides methods of screening for an agent that has inhibitory effect on P. acnes, wherein the methods comprise incubating P. acnes in the presence of an agent and detecting inhibitory effect of the agent on P. acnes.

In an eighth aspect of the present invention there is provided a method of preventing or treating a prostate disease in a subject, the method comprising administering to a subject in need thereof an effective amount of a P. acnes inhibitory composition.

In a ninth aspect of the present invention there is provided an isolated subtype of P. acnes Group 2 or Group 3 as hereinafter defined.

### BRIEF DESCRIPTION OF THE FIGURES

FIG. 1: shows a section of a whole mount prostatectomy specimen (hematoxylin and eosin (H&E), original magnification ×10) showing a background of glandular atrophy associated with stromal aggregates of lymphoid cells and histiocytes (Grade 1—chronic inflammation). Inserts (original magnification ×100) show acute (active) inflammation Grade 2, with migration of neutrophils through the gland wall (top right); concurrent high-grade dysplasia (PIN) (top left) and invasive carcinoma (bottom left).

FIG. 2: shows differences in growth properties and cell surface hydrophobicity for P. acnes of Group 1 compared to Group 2. Salt Aggregation Tests (left) show droplets of bacterial culture in increasing salt concentrations of 0.25M to 4M as well as a no-salt negative control (lop). Group 1 isolate (R3) has complete aggregation in 1M salt, indicating a hydrophobic cell surface compared to Group 2 isolate (2937) with a slight degree of aggregation in 2 and 4M salt. The cultures grown in liquid medium without agitation (right) show Group 1 isolate (R3) as a granular sediment with clear supernatant compared to Group 2 isolate (2937) which has a fine sediment with a turbid supernatant.

FIG. 3: shows a pulsed-field gel analysis of P. acnes isolates. Top panel (A), DNA banding patterns following digestion with SpeI; Bottom panel (B), DNA banding patterns following digestion with NotI. Groupings are based on similarity of DNA banding patterns, with Group 1 containing all cutaneous isolates (A1-R5) and two prostatic isolates (2703 and 2972). The remaining 10 prostatic P. acnes isolates form Groups 2 to 4.

FIG. 4: shows sequence variants of the P. acnes MMCoA gene. Sequences B (SEQ ID NO:1) and Sequence A (SEQ ID NO:2) differ at 21 specific single-base positions (each marked by an asterisk). Sequence B (SEQ ID NO:1) is polymorphic at two base positions (arrows) where either C or T may be present. Primers MMF (SEQ ID NO:3) and MMR (SEQ ID NO:4) amplify the region between nt 308 and nt 938.

FIG. 5; shows an example of DNA banding patterns obtained by RAPD-PCR of genomic DNA from P. acnes of Groups 1, 2 and 3 (two different isolates from each group are shown) using one RAPD primer. Panel A shows a band produced exclusively in Group 1 P. acnes (arrow), indicating the presence of DNA variations between Group 1 and Groups 2/3. Panel B shows a Southern Blot of this same region of DNA (SEQ ID NO:7 and SEQ ID NO:8) using a probe based on the DNA sequence from Group 1 P. acnes (nt 2165 to 2691 of SEQ ID NO:7). This region of DNA is shown to be missing from the genome of P. acnes Groups 2 and 3, indicating that the sequence variation is a large deletion.

FIG. 6: shows DNA sequence variations in the region of the Group 1 RAPD-PCR band containing the downstream end of a beta-lactamase gene (SEQ ID NO:13) compared to die corresponding sequence from Groups 2 and 3 (SEQ ID NO:14). Sequence differences are indicated by an asterisk. This region of DNA was used to design primers G2/3F1 (SEQ ID NO:15), G2/3F2 (SEQ ID NO:16) and G2/3R (SEQ ID NO; 17) that selectively amplify the region between nt 487 to nt 690 from P. acnes Groups 2 and/or 3.

FIG. 7: Organization of open reading frames (ORFs) within and adjacent to the 8692 nucleotide DNA region that is deleted from the genome of P. acnes Groups 2 and 3 (SEQ ID NO:7). Genomic regions within the box comprise the deleted region. Each ORF is represented by an arrow, with the arrow direction indicating the 5′ to 3′ direction of its coding sequence. The coding sequences of ORF X and ORF 1 overlap by 41 nucleotides. ORF X= putative Endo-beta-mannanase gene; ORF 1=N-acetyl-beta-hexosaminidase; ORF 2=ABC peptide transporter, permease component 1; ORF 3=ABC peptide transporter, permease component 2; ORF 4=ABC peptide transporter, ATP-binding component 1; ORF 5=ABC peptide transporter, ATP-binding component 2; ORF 6=ABC peptide transporter, solute-binding protein; ORF 7= Chitinase; ORF Y=Phosphopantetheine adenylyltransferase.

### DETAILED DESCRIPTION OF THE INVENTION

Accordingly, in a first aspect of the present invention there is provided a method of diagnosing the presence of, or the predisposition to develop, prostate disease in a subject, the method comprising analysing a test sample from the subject for the presence of P. acnes infection of the prostate gland.

Preferably, the step of analyzing a test sample for the presence of P. acnes is selected from the group consisting of bacterial nucleic acid (including DNA and RNA) analysis, protein analysts, culture analysis, antibody detection analysis, detection of P. acnes metabolic products and combinations thereof.

The prostate disease is preferably selected from the group consisting of prostatitis, dysplasia (pre-cancer) and prostate cancer.

In one embodiment, the presence of P. acnes that comprises the DNA sequence of SEQ ID NO:1, SEQ ID NO. 2, SEQ ID NO. 6, SEQ ID NO. 11, SEQ ID NO. 15, SEQ ID NO. 16, SEQ ID NO. 17, SEQ ID NO. 20, SEQ ID NO. 23, SEQ ID NO. 26, SEQ ID NO. 27, SEQ ID NO. 28, SEQ ID NO. 31, SEQ ID NO. 32, SEQ ID NO. 35, SEQ ID NO. 36, SEQ ID NO. 39, SEQ ID NO. 42, SEQ ID NO. 45 or SEQ ID NO. 46 is analyzed.

In another embodiment, the presence of P. acnes Group 2 and/or 3 is analysed by detecting a hydrophilic surface on the isolated bacterium in combination with SEQ ID NO:1, SEQ ID NO:6, SEQ ID NO:11, SEQ ID NO:16, SEQ ID NO:17, SEQ ID NO:27, SEQ ID NO:32, SEQ ID NO:36, SEQ ID NO:39, SEQ ID NO:42 or SEQ ID NO:46 but not including SEQ ID NO:10, SEQ ID NO:15, SEQ ID NO:20, SEQ ID NO:23, SEQ ID NO:31 or SEQ ID NO:35.

The term “test sample” used herein refers to a component of a subject's body from which the presence of P. acnes infection of the prostate can be detected. The test samples is preferably whole blood, serum, plasma, urine, semen, prostatic secretions or prostate tissue. Preferably, the test sample is urine.

The term “subject” used herein includes a mammal, preferably a human, more preferably a male. Mammals includes, but are not limited to, farm animals (such as cows), sport animals, pets (such as cats, dogs, horses), primates, mice and rats.

The presence of P. acnes Group 1 and/or Group 2 and/or Group 3 can be detected using any methods described herein and methods known in the art. For example, the presence of P. acnes can be detected by culturing methods (such as testing cell surface hydrophobicity by observing liquid growth characteristics and performing salt aggregation tests); detecting a DNA sequence that distinguishes P. acnes Group 1 and/or 2 and/or 3; detecting differential expression of RNAs and proteins that distinguish P. acnes Group 1 and/or 2 and/or 3; detecting the presence and/or absence of antigens using antibodies that distinguish P. acnes Group 1 and/or 2 and/or 3; detecting metabolic products that distinguish P. acnes Group 1 and/or 2 and/or 3; or by any combination of these methods.

In one embodiment, the analysis involves extraction of DNA from the test sample and amplification of P. acnes DNA present therein. It is presently preferred that the amplification of P. acnes DNA is achieved by PCR using primers specific to P. acnes, preferably P. acnes groups 2 and 3. It will, however, be recognized by persons skilled in the art that various other DNA amplification methods can equally be used in the present invention. For instance, DNA amplification methods can include, but are not limited to, ligation-based thermocycling approaches, real-time PCR, non-PCR isothermal DNA amplification techniques, for example: real-time strand displacement amplification (SDA), rolling-circle amplification (RCA) and multiple-displacement amplification (MDA).

In a further embodiment, the presence of P. acnes infection in prostate-gland is analyzed by detecting a DNA sequence that is specific for P. acnes, preferably a DNA sequence that distinguishes P. acnes Group 2 and/or Group 3 from Group 1. For example, the presence of P. acnes Group 2 and/or Group 3 can be demonstrated by detecting the presence of the DNA sequence set forth in SEQ ID NO:1, SEQ ID NO:6, SEQ ID NO:11, SEQ ID NO:16, SEQ ID NO:17, SEQ ID NO:27, SEQ ID NO:32, SEQ ID NO:36, SEQ ID NO:39, SEQ ID NO:42 and/or SEQ ID NO:46. The presence of P. acnes Group 2 and/or Group 3 can also be determined by any combination of these methods. Any primers and probes described herein may be used for detecting the presence, and/or absence of these sequences.

Other DNA sequences that distinguish P. acnes Group 2 and/or Group 3 from Group 1 may be identified using any methods known in the art. For example, random amplified polymorphic DNA (RAPD)-PCR, amplified fragment length polymorphism (AFLP), representational difference analysis (RDA). See, e.g., Vancanneyt (2002); Torriani (1999); Hou (1996); Lisitsyn (1995a); Lisitsyn (1995b). RAPD-PCR and RDA are also described in detail in Examples 5 and 10. DNA sequences specific for P. acnes Group 2 and/or Group 3 may be used to design PCR primers specific for Group 2 and/or Group 3. Examples of PCR primers suitable to distinguish Groups 1, 2 and 3 are outlined in Table 1.

In some embodiments, the presence of a DNA sequence that is not present in P. acnes Group 1 or that is specific for P. acnes Group 2 and/or Group 3 may be detected using non-PCR methods. For example, isothermal linear nucleic acid amplification methods for generating multiple copies of nucleic acid sequences of interest described in PCT WO 01/020035 and WO 2002/048402, U.S. Pat. No. 6,251,639, and U.S. Pat. No. 6,692,918 (which are hereby incorporated by reference in their entirety) may be used. A composite primer comprising an RNA portion and a 3′ DNA portion can be hybridized to a single stranded DNA template contacting the target sequence, and then the composite primer is extended with a DNA polymerase. Optionally, a termination polynucleotide sequence to a region of the template which is 5′ with respect hybridization of the composite primer to the template may be used. After the DNA polymerase extension, the RNA portion of the annealed composite primer is cleaved by an enzyme that cleaves RNA from an RNA/DNA hybrid such that another composite primer can hybridize to the template and repeat extension by strand displacement to generate multiple copies of the complementary sequence of the target sequence. The produced multiple copies of the complementary sequence of the target sequence may be hybridized to a sequence specific probe for detecting the presence of such specific sequence. The probe may be immobilized on a surface of a microarray. The composite primer may be derived from a sequence specific for P. acnes Group 2 and/or Group 3, or a sequence common to P. acnes but adjacent to the target sequence that is specific for P. acnes Group 2 and/or Group 3. For example, the composite primer may be derived regions of SEQ ID NO:11 or other sequences specific to P. acnes Group 2 and/or Group 3 that are not present in Group 1 P. acnes.

Other DNA detection technologies may also be used. For example, bio-bar-code (BCA) DNA detection, which provides high selectivity with a sensitivity that is comparable to PCR-based approaches without the need for enzymatic amplification may be used (Nam et al 2004). This method relics on two-component oligonucleotide-modified gold nanoparticles (NPs) and single-component oligonucleotide-modified magnetic microparticles (MMPs), and subsequent detection of amplified target DNA in the form of bar-code DNA using a chip-based detection method. The gold NPs are modified with oligonucleotides that are complementary to a region of a target sequence of interest and oligonucleotides complementary to a bar-code sequence that is a unique identification tag for the target sequence. The MMPs are functionalized with oligonucleotides that are complementary to a region of the target sequence different from the NPs. When the target is present in the system, the target can be sandwiched with MMP and NP probes to form a complex. Magnetic separation of the complex followed by identification of the bar-code DNA dehybridized allows determination of the presence of the target sequence. Because the NP probe carries with it a large number of bar-code complementary oligonucleotides per target sequence complementary oligonucleotides, there is substantial amplification of the signal.

In another embodiment, the presence of P. acnes is analyzed by detecting gene expression specific for P. acnes or specifically expressed in P. acnes. Preferably, the presence of P. acnes Group 2 and/or Group 3 is analyzed, e.g., by detecting differential expression of a gene mat distinguishes P. acnes Group 2 and/or Group 3 from Group 1.

In other embodiments, the presence of P. acnes Group 1 and/or 2 and/or 3 is analyzed by detecting differential expression of an RNA that distinguishes P. acnes Group 1 and/or 2 and/or 3.

In other embodiments, the presence of P. acnes Group 1 and/or 2 and/or 3 is analyzed by detecting differential expression of a protein that distinguishes P. acnes Group 1 and/or 2) and/or 3. In some embodiments, the protein detected is a cell surface protein.

In other embodiments, the presence of P. acnes is analyzed by detecting differential expression of an antigen that distinguishes P. acnes Group 1 and/or 2 and/or 3. In some embodiments, differential expression of the antigen that distinguishes P. acnes Group 1 and/or 2 and/or 3 is detected using an antibody that specifically binds to P. acnes Group 1 and/or 2 and/or 3. In some embodiments, the antigen is a cell surface antigen.

In some embodiments, the presence of P. acnes Group 1 and/or 2 and/or 3 is analyzed by any combination of the methods described herein.

“Differential expression” used herein, refers to both qualitative as well as quantitative differences in the temporal and/or cellular expression patters of genes, within and among the cells. Expression profile of a specific group of P. acnes at a particular stage or growth cycle may be established. Thus, a differentially expressed gene can qualitatively have its expression altered, including an activation or inactivation, in, for example, in P. acnes Group 2 and/or Group 3 versus in Group 1. As is apparent to the skilled artisan, any comparison can be made. Such a qualitatively regulated gene will exhibit an expression pattern within one group, which is detectable by standard techniques in one such group, but is not detectable in the other group. Alternatively, the determination is quantitative in that expression is increased or decreased; that is, the expression of the gene is either up-regulated, resulting in an increased amount of transcript, or down-regulated, resulting in a decreased amount of transcript. The degree to which expression differs need only be large enough to quantify via standard characterization techniques as outlined below, such as by use of Affymetrix GeneChip® expression arrays, Lockhart (1996), hereby expressly incorporated by reference. Other techniques include, but are not limited to, quantitative reverse transcriptase PCR, Northern analysis and RNase protection. As outlined above, preferably the change in expression (i.e. upregulation or downregulation) is at least about 50%, more preferably at least about 100%, more preferably at least about 150%, more preferably, at least about 200%, with from 300 to at least 1000% being especially preferred.

As will be appreciated by those in the art, this may be done by evaluation at either the gene transcript, or the protein level; that is, the amount of gene expression may be monitored using nucleic acid probes to the DNA or RNA equivalent of the gene transcript, and the quantification of gene expression levels, or, alternatively, the final gene product itself (protein) can be monitored, for example through the use of antibodies to the P. acnes protein and standard immunoassays (ELISAs, etc.) or other techniques, including mass spectroscopy assays, 2D get electrophoresis assays, etc.

In another embodiment, the presence of P. acnes is analyzed by culture analysis. The sample is added to culture media and incubated. The culture media is then analyzed for the presence of P. acnes, preferably P. acnes Groups 2 and/or 3. The presence of P. acnes having hydrophilic surface (Group 2 and Group 3), comparing to hydrophobic surface (Group 1) can be analyzed. The bacterial cell surface hydrophobicity may be tested using any methods known in the art, such as observing growth characteristics and salt aggregation tests (described in detail in Example 2). Culture media that can be used for culturing P. acnes are known in the art. An example of such culture media and culture condition is described in detail in Example 8.

In a further preferred embodiment the sample is analyzed for the presence of antibodies specific to P. acnes, preferably P. acnes Groups 2 and/or 3. Any immunoassays known in the art may be used.

In some embodiments of the present invention, the analysis comprises

(a) contacting the test sample with at least one P. acnes antibody which specifically binds to at least one epitope of the P. acnes antigen for a time and under conditions sufficient for the formation of antibody/antigen complexes; and
(b) detecting the presence of the antibody/antigen complex as an indication of the presence of P. acnes antigen in the test sample.

Preferably the antibody binds specifically to P. acnes Groups 2 and/or 3.

In a second aspect of the present invention there is provided an isolated polynucleotide, wherein the isolated polynucleotide has a sequence selected from the group consisting of:

### SEQ ID NO:1;

a sequence at least 99% identical to SEQ ID NO:1;
a fragment of at least about 10, at least about 15, at least about 20, at least about 25, at least about 30, at least about 50, at least about 100, at least about 150, at least about 200or more contiguous nucleotides of SEQ ID NO:1, wherein the sequence comprises a sequence that is not present in P. acnes Group 1 or that is specific for P. acnes Group 3;
a sequence which hybridizes to SEQ ID NO:1 under conditions of high stringency;
a sequence complementary to SEQ ID NO:1 or a fragment of SEQ ID NO:1 described above. SEQ ID NO:1;

### SEQ ID NO:2;

a sequence at least 99% identical to SEQ ID NO:2;
a fragment of at least about 10, at least about 15, at least about 20, at least about 25, at 5 least about 30, at least about 50, at least about 100, at least about 150, at least about 200or more contiguous nucleotides of SEQ ID NO:2, wherein the sequence comprises a sequence that is not present in P. acnes Group 3 or that is specific for P. acnes Group 1 or 2;
a sequence which hybridizes to SEQ ID NO:2 under conditions of high stringency;
a sequence complementary to SEQ ID NO:2 or a fragment of SEQ ID NO:2 described above.

### SEQ ID NO:6;

a sequence at least 95%, preferably at least 96%, more preferably at least 97%, even more preferably at least 98%, and more preferably at least 99% identical to SEQ ID NO:6;
a fragment of at least about 10, at least about 15, at least about 20, at least about 25, at least about 30, at least about 50, at least about 100, at least about 150, at least about 200or more contiguous nucleotides of SEQ ID NO:6, wherein the sequence comprises a sequence that is not present in P. acnes Group 1 or that is specific for P. acnes Group 2 and/or Group 3;
a sequence which hybridizes to SEQ ID NO:6 under conditions of high stringency;
a sequence complementary to SEQ ID NO:6 or a fragment of SEQ ID NO:6 described above;

### SEQ ID NO:11;

a sequence at least 95%, preferably at least 96%, more preferably at least 97%, even more preferably at least 98%, and more preferably at least 99% identical to SEQ ID NO:11;
a fragment of at least about 10, at least about 15, at least about 20, at least about 25, at least about 30, at least about 50, at least about 100, at least about 150, at least about 200or more contiguous nucleotides of SEQ ID NO:11, wherein the fragment comprises a sequence that is not present in P. acnes Group 1 or that is specific for P. acnes Group 2 and/or Group 3;
a sequence which hybridizes to SEQ ID NO:11 under conditions of high stringency;
a sequence complementary to SEQ ID NO:11 or a fragment of SEQ ID NO:11 described above;

### SEQ ID NO:15;

a sequence at least 95%, preferably at least 96%, more preferably at least 97%, even more preferably at least 98%, and more preferably at least 99% identical to SEQ ID NO:15;
a fragment of at least 10, at least 15, at least 20, at least 25, at least 30, at least 50, at least 100, at least 150, at least 200 or more contiguous nucleotides of SEQ ID NO:15, wherein the sequence comprises a sequence that is not present in P. acnes Group 2 and/or Group 3 or that is specific for P. acnes Group 1;
a sequence which hybridizes to SEQ ID NO:15 under conditions of high stringency;
a sequence complementary to SEQ ID NO:15 or a fragment of SEQ ID NO:15 described above;

### SEQ ID NO:16;

a sequence at least 95%, preferably at least 96%, more preferably at least 97%, even more preferably at least 98%, and more preferably at least 99% identical to SEQ ID NO:16;
a fragment of at least 10, at least 15, at least 20, at least 25, at least 30, at least 50, at least 100, at least 150, at least 200 or more contiguous nucleotides of SEQ ID NO:16, wherein the sequence comprises a sequence that is not present in P. acnes Group 1 and/or Group 3 or that is specific for P. acnes Group 2;
a sequence which hybridizes to SEQ ID NO:16 under conditions of high stringency;
a sequence complementary to SEQ ID NO:16 or a fragment of SEQ ID NO:16 described above;

### SEQ ID NO:17;

a sequence at least 95%, preferably at least 96%, more preferably at least 97%, even more preferably at least 98%, and more preferably at least 99% identical to SEQ ID NO:17;
a fragment of at least 10, at least 15, at least 20, at least 25, at least 30, at least 50, at least 100, at least 150, at least 200 or more contiguous nucleotides of SEQ ID NO:17, wherein the sequence comprises a sequence that is not present in P. acnes Group 1 and/or Group 2 or that is specific for P. acnes Group 3;
a sequence which hybridizes to SEQ ID NO:17 under conditions of high stringency;
a sequence complementary to SEQ ID NO:17 or a fragment of SEQ ID NO:17 described above;

### SEQ ID NO:20;

a sequence at least 95%, preferably at least 96%, more preferably at least 97%, even more, preferably at least 98%, and more preferably at least 99% identical to SEQ ID NO:20;
a fragment of at least 10, at least 15, at least 20, at least 25, at least 30, at least 50, at least 100, at least 150, at least 200 or more contiguous nucleotides of SEQ ID NO:20, wherein the sequence comprises a sequence that is not present in P. acnes Group 2 and/or Group 3or that is specific for P. acnes Group 1;
a sequence which hybridizes to SEQ ID NO:20 under conditions of high stringency;
a sequence complementary to SEQ ID NO:20 or a fragment of SEQ ID NO:20 described above;

### SEQ ID NO:23;

a sequence at least 95%, preferably at least 96%, more preferably at least 97%, even more preferably at least 98%, and more preferably at least 99% identical to SEQ ID NO:23;
a fragment of at least 10, at least 15, at least 20, at least 25, at least 30, at least 50, at least 100, at least 150, at least 200 or more contiguous nucleotides of SEQ ID NO:23, wherein the sequence comprises a sequence that is not present in P. acnes Group 2 and/or Group 3or that is specific for P. acnes Group 1;
a sequence which hybridizes to SEQ ID NO:23 under conditions of high stringency;
a sequence complementary to SEQ ID NO:23 or a fragment of SEQ ID NO:23 described above;

### SEQ ID NO:26;

a sequence at least 95%, preferably at least 96%, more preferably at least 97%, even more preferably at least 98%, and more preferably at least 9.9% identical to SEQ ID NO:26;
a fragment of at least 10, at least 15, at least 20, at least 25, at least 30, at least 50, at least 100, at least 150, at least 200 or more contiguous nucleotides of SEQ ID NO:26, wherein the sequence comprises a sequence that is not present in P. acnes Group 2 and/or Group 3or that is specific for P. acnes Group 1;
a sequence which hybridizes to SEQ ID NO:26 under conditions of high stringency;
a sequence complementary to SEQ ID NO:23 or a fragment of SEQ ID NO:23 described above;

### SEQ ID NO:27;

a sequence at least 95%, preferably at least 96%, more preferably at least 97%, even more preferably at least 98%, and more preferably at least 99% identical to SEQ ID NO:27;
a fragment of at least 10, at least 15, at least 20, at least 25, at least 30, at least 50, at least 100, at least 150, at least 200 or more contiguous nucleotides of SEQ ID NO:27, wherein the sequence comprises a sequence that is not present in P. acnes Group 1 and/or Group 3or that is specific for P. acnes Group 2;
a sequence which hybridizes to SEQ ID NO:27 under conditions of high stringency;
a sequence complementary to SEQ ID NO:27 or a fragment of SEQ ID NO:27 described above;

### SEQ ID NO:28;

a sequence at least 95%, preferably at least 96%, more preferably at least 97%, even more preferably at least 98%, and more preferably at least 99% identical to SEQ ID NO:28;
a fragment of at least 10, at least 15, at least 20, at least 25, at least 30, at least 50, at least 100, at least 150, at least 200 or more contiguous nucleotides of SEQ ID NO:28, wherein the sequence comprises a sequence that is not present in P. acnes Group 1 and/or Group 2 or that is specific for P. acnes Group 3;
a sequence which hybridizes to SEQ ID NO:28 under conditions of high stringency;
a sequence complementary to SEQ ID NO:28 or a fragment of SEQ ID NO:28 described above;

### SEQ ID NO:31;

a sequence at least 95%, preferably at least 96%, more preferably at least 97%, even more preferably at least 98%, and more preferably at least 99% identical to SEQ ID NO:31;
a fragment of at least 10, at least 15, at least 20, at least 25, at least 30, at least 50, at least 100, at least 150, at least 200 or more contiguous nucleotides of SEQ ID NO:31, wherein the sequence comprises a sequence that is not present in P. acnes Group 2 and/or Group 3or that is specific for P. acnes Group 1;
a sequence which hybridizes to SEQ ID NO:31 under conditions of high stringency;
a sequence complementary to SEQ ID NO:31 or a fragment of SEQ ID NO:31 described above;

### SEQ ID NO:32;

a sequence at least 95%, preferably at least 96%, more preferably at least 97%, even more preferably at least 98%, and more preferably at least 99% identical to SEQ ID NO:32;
a fragment of at least 10, at least 15, at least 20, at least 25, at least 30, at least 50, at least 100, at least 150, at least 200 or more contiguous nucleotides of SEQ ID NO:32, wherein the sequence comprises a sequence that is not present in P. acnes Group 1 and/or Group 3 or that is specific for P. acnes Group 2;
a sequence which hybridizes to SEQ ID NO:32 under conditions of high stringency;
a sequence complementary to SEQ ID NO:32 or a fragment of SEQ ID NO:32 described above;

### SEQ ID NO:35;

a sequence at least 95%, preferably at least 96%, more preferably at least 97%, even more preferably at least 98%, and more preferably at least 99% identical to SEQ ID NO:35;
a fragment of at least 10, at least 15, at least 20, at least 25, at least 30, at least 50, at least 100, at least 150, at least 200 or more contiguous nucleotides of SEQ ID NO:35, wherein the sequence comprises a sequence that is not present in P. acnes Group 2 and/or Group 3or that is specific for P. acnes Group 1;
a sequence which hybridizes to SEQ ID NO:35 under conditions of high stringency;
a sequence complementary to SEQ ID NO:35 or a fragment of SEQ ID NO:35 described above;

### SEQ ID NO:36;

a sequence at least 95%, preferably at least 96%, more preferably at least 97%, even more preferably at least 98%, and more preferably at least 99% identical to SEQ ID NO:36;
a fragment of at least 10, at least 15, at least 20, at least 25, at least 30, at least 50, at least 100, at least 150, at least 200 or more contiguous nucleotides of SEQ ID NO:36, wherein the sequence comprises a sequence that is not present in P. acnes Group 1 and/or Group 3or that is specific for P. acnes Group 2;
a sequence which hybridizes to SEQ ID NO:36 under conditions of high stringency;
a sequence complementary to SEQ ID NO:36 or a fragment of SEQ ID NO:36 described above;

### SEQ ID NO:39;

a sequence at least 95%, preferably at least 96%, more preferably at least 97%, even more preferably at least 98%, and more preferably at least 99% identical to SEQ ID NO:39;
a fragment of at least 10, at least 15, at least 20, at least 25, at least 30, at least 50, at least 100, at least 150, at least 200 or more contiguous nucleotides of SEQ ID NO:39, wherein the sequence comprises a sequence that is not present in P. acnes Group 1 and/or Group 3or that is specific for P. acnes Group 2;
a sequence which hybridizes to SEQ ID NO:39 under conditions of high stringency;
a sequence complementary to SEQ ID NO:39 or a fragment of SEQ ID NO:39 described above;

### SEQ ID NO:42;

a sequence at least 95%, preferably at least 96%, more preferably at least 97%, even more preferably at least 98%, and more preferably at least 99% identical to SEQ ID NO:42;
a fragment of at least 10, at least 15, at least 20, at least 25, at least 30, at least 50, at least 100, at least 150, at least 200 or more contiguous nucleotides of SEQ ID NO:42, wherein the sequence comprises a sequence that is not present in P. acnes Group 1 and/or Group 3or that is specific for P. acnes Group 2;
a sequence which hybridizes to SEQ ID NO:42 under conditions of high stringency;
a sequence complementary to SEQ ID NO:42 or a fragment of SEQ ID NO:42 described above;

### SEQ ID NO:45;

a sequence at least 95%, preferably at least 96%, more preferably at least 97%, even more preferably at least 98%, and more preferably at least 99% identical to SEQ ID NO:45;
a fragment of at least 10, at least 15, at least 20, at least 25, at least 30, at least 50, at least 100, at least 150, at least 200 or more contiguous nucleotides of SEQ ID NO:45, wherein the sequence comprises a sequence that is not present in P. acnes Group 3 that is specific for P. acnes Group 1 or Group 2;
a sequence which hybridizes to SEQ ID NO:45 under conditions of high stringency;
a sequence complementary to SEQ ID NO:45 or a fragment of SEQ ID NO:45 described above;

### SEQ ID NO:46;

a sequence at least 95%, preferably at least 96%, more preferably at least 97%, even more preferably at least 98%, and more preferably at least 99% identical to SEQ ID NO:46;
a fragment of at least 10, at least 15, at least 20, at least 25, at least 30, at least 50, at least 100, at least 150, at least 200 or more contiguous nucleotides of SEQ ID NO:46, wherein the sequence comprises a sequence that is not present in P. acnes Group 1 or Group 2 that is specific for P. acnes Group 3;
a sequence which hybridizes to SEQ ID NO:46 under conditions of high stringency;
a sequence complementary to SEQ ID NO:46 or a fragment of SEQ ID NO:46 described above.

Polynucleotide sequences specific to P. acnes are useful as primers for the amplification of DNA or as probes to determine the presence of P. acnes nucleic acid sequences in test samples.

In a third aspect, the present invention provides a primer wherein the primer binds specifically to a polynucleotide according to the second aspect of the invention.

A “primer” is generally a short single stranded polynucleotide, generally with a free 3′-OH group, that binds to a target potentially present in a sample of interest by hybridizing with a target sequence, and thereafter promotes polymerization of a polynucleotide complementary to the target. A primer may be about 10, 15, 20, 25, 30, 35, 40, 45, 50 or more nucleotides in length.

As used herein, “polynucleotide,” or “nucleic acid,” as used interchangeably herein, refer to polymers of nucleotides of any length (such as at least 10 nt, 15 nt, 20 nt, 30 nt, 50 nt, 75 nt, 100 nt, 150 nt, 200 nt, or longer), and include DNA and RNA. The nucleotides can be deoxyribonucleotides, ribonucleotides, modified nucleotides or bases, and/or then analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

For the purpose of this invention, the “sequence identity” of two related nucleotide or amino acid sequences, expressed as a percentage, refers to the number of positions in the two optimally aligned sequences which have identical residues (×100) divided by the (number of positions compared. A gap, i.e., a position in an alignment where a residue is present in one sequence but not in the other is regarded as a position with non-identical residues. The alignment of the two sequences is performed by the Needleman and Wunsch algorithm (Needleman and Wunsch 1970). The computer-assisted sequence alignment above can be conveniently performed using standard software program such as GAP, which is part of the Wisconsin Package Version 10.1 (Genetics Computer Group, Madison, Wis., USA) using the default scoring matrix with a gap creation penalty of 50 and a gap extension penalty of 3. It is clear that when RNA sequences are to be essentially similar or have a certain degree of sequence identity with DNA sequences, thymine (T) in the DNA sequence is considered equal to uracil (U) in the RNA sequence.

As used herein, stringent conditions are those that (1) employ low ionic strength and high temperature for washing, for example, 0.015 M NaCl/0.0015 M sodium citrate/0.1% NaDodSO₄ at 50° C.; (2) employ during hybridization a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS. These conditions are examples of high stringency conditions. The skilled artisan will recognize how to adjust the temperature, ionic strength, etc. as necessary to accommodate factors such as probe length and the like. Sec, “Molecular Cloning: A Laboratory Manual”, second edition (Sambrook et al., 1989)

In a fourth aspect, the present invention provides a primer sequence that distinguishes between Group 1, Group 2 and Group 3 P. acnes as herein defined.

In a preferred embodiment, the primer sequence is based on SEQ ID NO. 1, SEQ ID NO. 2, SEQ ID NO. 6, SEQ ID NO. 11, SEQ ID NO. 15, SEQ ID NO. 16, SEQ ID NO. 17, SEQ ID NO. 20, SEQ ID NO. 23, SEQ ID NO. 26, SEQ ID NO. 27, SEQ ID NO. 28, SEQ ID NO. 31, SEQ ID NO. 32, SEQ ID NO. 35, SEQ ID NO. 36, SEQ ID NO. 39, SEQ ID NO. 42, SEQ ID NO. 45 or SEQ ID NO. 46 or other regions of the P. acnes genome that can identify P. acnes and distinguish Group 1 and/or 2 and/or 3.

The present invention also provides pairs of primers for detecting the presence of P. acnes Group 1 and/or Group 2 and/or Group 3 in a sample. In some embodiments, at least one of the primers specifically binds to SEQ ID NO. 1, SEQ ID NO. 2, SEQ ID NO. 6, SEQ ID NO. 11, SEQ ID NO. 15, SEQ ID NO. 16, SEQ ID NO. 17, SEQ ID NO. 20, SEQ ID NO. 23, SEQ ID NO. 26, SEQ ID NO. 27, SEQ ID NO. 28, SEQ ID NO. 31, SEQ ED NO. 32, SEQ ID NO. 35, SEQ ID NO. 36, SEQ ID NO. 39, SEQ ID NO. 42, SEQ ID NO. 45 and SEQ ID NO. 46 or to a sequence that is at least 95%, at least 96%, at least 97%, at least 98%, or at least 99% identical to SEQ ID NO. 1, SEQ ID NO. 2, SEQ ID NO. 6, SEQ ID NO. 11, SEQ ID NO. 15, SEQ ID NO. 16, SEQ ID NO. 17, SEQ ID NO. 20, SEQ ID NO. 23, SEQ ID NO. 26, SEQ ID NO. 27, SEQ ID NO. 28, SEQ ID NO. 31, SEQ ID NO. 32, SEQ ID NO. 35, SEQ ID NO. 36, SEQ ID NO. 39, SEQ ID NO. 42, SEQ ID NO. 45 and SEQ ID NO. 46 or other regions of the P. acnes genome that can identify P. acnes and distinguish Group 1 and/or 2 and/or 3.

In a preferred embodiment, the primer is selected from the group consisting of SEQ ID NO. 3, SEQ ID NO. 4, SEQ ID NO. 7, SEQ ID NO. 8, SEQ ID NO. 9, SEQ ID NO. 12, SEQ ID NO. 13, SEQ ID NO. 14, SEQ ID NO. 18, SEQ ID NO. 19, SEQ ID NO. 21, SEQ ID NO. 22, SEQ ID NO. 24, SEQ ID NO. 25, SEQ ID NO. 29, SEQ ID NO. 30, SEQ ID NO. 33, SEQ ID NO. 34, SEQ ID NO. 37, SEQ ID NO. 38, SEQ ID NO. 40, SEQ ID NO. 41, SEQ ID NO. 43, SEQ ID NO. 44, SEQ ID NO. 47 and SEQ ID NO. 48.

In some embodiments, die presence of P. acnes Group 1, 2 or 3 is detected by the presence of the DNA sequence set forth in SEQ ID NO:1 or SEQ ID NO:2. In some embodiments, the presence of the DNA sequence set forth in SEQ ID NO:1 or SEQ ID NO:2 is detected by amplification performed using PCR with primer pairs MMF (SEQ ID NO:3) and MMR (SEQ ID NO:4) and assessing the presence or absence of a specific nucleotide sequence in the amplified PCR product, where the presence of SEQ ID NO:1 indicates P. acnes Group 3 and SEQ ID NO:2 indicates P. acnes Group 1 or 2.

In some embodiments, the presence of P. acnes Group 2 and/or Group 3 is analyzed by detecting a DNA sequence that distinguishes P. acnes Group 2 and/or Group 3 from Group 1. In some embodiments, the presence of P. acnes Group 2 and/or Group 3 is detected by the presence of the DNA sequence set forth in SEQ ID NO:6. In some embodiments, the presence of the DNA sequence set forth in SEQ ID NO:6 is detected by amplification using PCR with primer pairs G2/3F1 (SEQ ID NO:7) and G2/3R (SEQ ID NO:8), and/or G2/3F2 (SEQ ID NO:9) and G2/3R (SEQ ID NO:8) and assessing the presence or absence of the PCR product and/or the nucleotide sequence of the amplified PCR product that is specific for P. acnes Group 2 and/or Group 3.

In some embodiments, the presence of P. acnes Group 2 and/or Group 3 is analyzed by detecting the presence of the DNA sequence set forth in SEQ ID NO:11. In some embodiments, the presence of the DNA sequence set forth in SEQ ID NO:11 is detected by amplification performed using PCR with primer pairs DEEP (SEQ ID NO:12) and DELR1 (SEQ ID NO:13) and/or DELE (SEQ ID NO:12) and DELR2 (SEQ ID NO:14), and assessing the presence and/or the nucleotide sequence of the amplified PCR product that is specific for P. acnes Group 2 and/or Group 3 (SEQ ID NO:11).

In some embodiments, the presence of P. acnes Group 2 and/or Group 3 is analyzed by detecting the deletion of the DNA sequence of about 8.7 kb, which encodes the following open reading frames: N-acetyl-beta-hexosaminidase; ABC peptide transporter, permease component 1; ABC peptide transporter, permease component 2; ABC peptide transporter, ATP-binding component 1; ABC peptide transporter, ATP-binding component 2; ABC peptide transporter, solute-binding protein; and Chitinase, and is set forth in SEQ ID NO:10. The absence of the DNA sequence as set out in can be analyzed by any method of amplification or hybridization.

In some embodiments, the presence of P. acnes Group 1 is analyzed by the inability to amplify the DNA sequence set forth in SEQ ID NO:11. In some embodiments, the inability to amplify the DNA sequence set forth in SEQ ID NO:11 using PCR with primer pairs DELF (SEQ ID NO:12) and DELR1 (SEQ ID NO:13) and/or DELF (SEQ ID NO:12) and DELR2 (SEQ ID NO:14), is due to the presence of about 8.7 kb of DNA sequence as set out in SEQ ID NO:10.

In further embodiments, the presence of P. acnes Group 1 is analyzed by detecting the presence of the DNA sequence set forth in SEQ ID NO:10 by any method of amplification or hybridization.

In some embodiments, the presence of P. acnes Group 2 and/or Group 3 is detected by the presence of the DNA sequence set forth in SEQ ID NO:16 and SEQ ID NO:17 respectively that distinguishe P. acnes Group 2 and Group 3 from Group 1. In some embodiments, the presence of P. acnes Group 2 and Group 3 is detected by the presence of the DNA sequence set forth in SEQ ID NO:16 and SEQ ID NO:17 and P. acnes Group 1 is detected by the presence of the DNA sequence set forth in SEQ ID NO:15. In some embodiments, the presence of the DNA sequence set forth in SEQ ID NO:15, 16 and 17 is detected by amplification using PCR with primer pairs PR262 (SEQ ID NO:18) and PR263 (SEQ ID NO:19), and assessing the presence of the PCR product of the specified size and/or the nucleotide sequence of the amplified PCR product that is specific for P. acnes Group 1 and Group 2 or 3.

In some embodiments, the presence of P. acnes Group 1 is detected by the presence of the DNA sequence set forth in SEQ ID NO:20 that distinguishes P. acnes Group 1 from Groups 2 and 3. In some embodiments, the presence of the DNA sequence set forth in SEQ ID NO:20 is detected by amplification performed using PCR with primer pairs PR090 (SEQ ID NO:21) and PR108 (SEQ DD NO:22), and assessing the presence or absence of the PCR product of the specified size and/or the nucleotide sequence of the amplified PCR product that is specific for P. acnes Group 1. No amplified product is produced for P. acnes Group 2 or 3.

In some embodiments, the presence of P. acnes Group 1 is detected by the presence of the DNA sequence set forth in SEQ ID NO:23 that distinguishes P. acnes Group 1 from Groups 2 and 3. In some embodiments, the presence of the DNA sequence set forth in SEQ ID NO:23 is detected by amplification performed using PCR with primer pairs PR213 (SEQ ID NO:24) and PR216 (SEQ ID NO:25), and assessing the presence or absence of the PCR product of the specified size and/or the nucleotide sequence of the amplified PCR product that is specific for P. acnes Group 1. No amplified product is produced for P. acnes Group 2 or 3.

In some embodiments, the presence of P. acnes Group 2 is detected by the presence of the DNA sequence set forth in SEQ ID NO:27 that distinguishes P. acnes Group 2 from Groups 1 and 3. In some embodiments, the presence of P. acnes Group 2 is detected by die presence of the DNA sequence set forth in SEQ ID NO:27 and P. acnes Group 1 and 3 are detected by the presence of the DNA sequence set forth in SEQ ID NO:26 and SEQ ID NO:28 respectively. In some embodiments, the presence of the DNA sequence set forth in SEQ ID NO:26, 27 and 28 is detected by amplification using PCR with primer pairs PR217 (SEQ ID NO:29) and PR218 (SEQ ID NO:30), and assessing the presence of the PCR product of the specified size and/or the nucleotide sequence of the amplified PCR product that is specific for P. acnes Group 2 and Group 1 or 3.

In some embodiments, the presence of P. acnes Group 2 is detected by the presence of the DNA sequence set forth in SEQ ID NO:32 that distinguishes P. acnes Group 2 from Groups 1 and Group 3. In some embodiments, the presence of P. acnes Group 2 is detected by the presence of the DNA sequence set forth in SEQ ID NO:32 and P. acnes Group 1 is detected by the presence of the DNA sequence set forth in SEQ ID NO:31. In some embodiments, the presence of the DNA sequence set forth in SEQ ID NO:32 and SEQ ID NO:31 is detected by amplification using PCR with primer pairs PR219 (SEQ ID NO:33) and PR220 (SEQ ID NO:34), and assessing the presence or absence of the PCR product of the specified size and/or the nucleotide sequence of the amplified PCR product that is specific for P. acnes Group 1 and Group 2 with no product visible for Group 3.

In some embodiments, the presence of P. acnes Group 2 is detected by the presence of the DNA sequence set forth in SEQ ID NO:36 that distinguishes P. acnes Group 2 from Groups 1 and Group 3. In some embodiments, the presence of P. acnes Group 2 is detected by the presence of the DNA sequence set forth in SEQ ID NO:36 and P. acnes Group 1 is detected by the presence of the DNA sequence set forth in SEQ ID NO:35. In some embodiments, the presence of the DNA sequence set forth in SEQ ID NO:36 and SEQ ID NO:35 is detected by amplification using PCR with primer pairs PR221 (SEQ ID NO:37) and PR222 (SEQ ID NO:38), and assessing the presence or absence of the PCR product of the specified size and/or the nucleotide sequence of the amplified PCR product that is specific for P. acnes Group 1 and Group 2 with no product visible for Group 3.

In some embodiments, the presence of P. acnes Group 2 is detected by the presence of the DNA sequence set forth in SEQ ID NO:39 that distinguishes P. acnes Group 2 from Groups 1 and Group 3. In some embodiments, the presence of the DNA sequence set forth in SEQ ID NO:39 is detected by amplification using PCR with primer pairs PR256 (SEQ ID NO:40) and PR257 (SEQ ID NO:41), and assessing the presence or absence of the PCR product of the specified size and/or the nucleotide sequence of the amplified PCR product that is specific for P. acnes Group 2 with no product visible for Group 1 and Group 3.

In some embodiments, the presence of P. acnes Group 2 is detected by the presence of the DNA sequence set forth in SEQ ID NO:42 that distinguishes P. acnes Group 2 from Groups 1 and Group 3. In some embodiments, the presence of the DNA sequence set forth in SEQ ID NO:42 is detected by amplification using PCR with primer pairs PR253 (SEQ ID NO:43) and PR254 (SEQ ID NO:44), and assessing the presence or absence of the PCR product of the specified size and/or the nucleotide sequence of the amplified PCR product that is specific for P. acnes Group 2 with no product visible for Group 1 and Group 3.

In some embodiments, the presence of P. acnes Group 3 is detected by the presence of the DNA sequence set forth in SEQ ID NO:46 that distinguishes P. acnes Group 3 from Groups 1 and 2. In some embodiments, the presence of P. acnes Group 3 is detected by the presence of the DNA sequence set forth in SEQ ID NO:46 and P. acnes Group 1 and 2 are detected by the presence of the DNA sequence set forth in SEQ ID NO:45. In some embodiments, the presence of the DNA sequence set forth in SEQ ID NO:46 and SEQ ID NO:45 is detected by amplification using PCR with primer pairs PR245 (SEQ ID NO:47) and PR247 (SEQ ID NO:48), and assessing the presence or absence of the PCR product of the specified size and/or the nucleotide sequence of the amplified PCR product that is specific for P. acnes Group 3 and Groups 1 and 2.

In a preferred embodiment, the DNA amplification is conducted by PCR using polynucleotide primers described in table 1. The primer may comprise sequences that are common in different groups of P. acnes, such as Groups 1, 2, and 3, but amplify a DNA sequence specific for P. acnes Group 1 and/or Group 2 and/or Group 3. Primers may comprise P. acnes Group 1 and/or Group 2 and/or Group 3 specific sequences and selectively amplify P. acnes Group 1 and/or Group 2 and/or Group 3 sequences. Any primers described herein may be used. It is preferred that following amplification the amplification product is analyzed, preferably by gel analysis, sequencing, by single strand conformational polymorphism (SSCP), hybridizing to an oligonucleotide probe immobilized on a surface, or other method of PCR product analysis

In a further preferred embodiment, the analysis involves PCR primers that can selectively amplify specific sized products from Groups 2 and/or 3 P. acnes and not other groups of P. acnes. It is preferred that this amplification is conducted by PCR using primer polynucleotide sequences within the region defined by SEQ ID NO. 1, SEQ ID NO. 6, SEQ ID NO. 11, SEQ ID NO. 16, SEQ ID NO. 17, SEQ ID NO. 27, SEQ ID NO. 32, SEQ ID NO. 36, SEQ ID NO. 39, SEQ ID NO. 42, SEQ ID NO. 46, or other such primer sequences that can distinguish Groups 2 and 3 P. acnes from Group 1 P. acnes. In a preferred embodiment, the PCR primers are SEQ ID NO:3 and SEQ ID NO:4, SEQ ID NO:7 and SEQ ID NO:8, SEQ ID NO:9 and SEQ ID NO:8, SEQ ID NO:12 and SEQ ID NO:13, SEQ ID NO:12 and SEQ ID NO:14, SEQ ID NO:18 and SEQ ID NO: 19, SEQ ID NO:29 and SEQ ID NO:30, SEQ ID NO:33 and SEQ ID NO:34, SEQ ID NO:37 and SEQ ID NO:38, SEQ ID NO:40 and SEQ ID NO:41, SEQ ID NO:43 and SEQ ID NO:44 and/or SEQ ID NO:47 and SEQ ID NO:48. These PCR products can be assessed by gel analysis or other method of PCR product evaluation, such as microarrays. The presence of a specific sequence may be detected using a specific probe described herein,

TABLE 1

| | Primer | Product produced with Primers | | |
|---|---|---|---|---|
| Primers for PCR | Names | Group 1 | Group 2 | Group 3 |
| SEQ ID NO: 3 and SEQ ID NO: 4 | MMF, MMR | 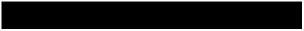 | | SEQ ID NO: 1 (633 bp) |
| SEQ ID NO: 7 and SEQ ID NO: 6SEQ ID NO: 9 and SEQ ID NO: 8SEQ ID NO: 12 and SEQ ID NO: 13SEQ ID NO: 12 and SEQ ID NO: 14SEQ ID NO: 18 and SEQ ID NO: 19 | G273F1, G273RG273F2, G273RDELF, DELR1DELF, DELR2PR262, PR263 | SEQ ID NO: 15 (901 bp) | 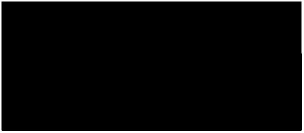 | |
| SEQ ID NO: 21 and SEQ ID NO: 22SEQ ID NO: 24 and SEQ ID NO: 25 | PR090, PR108PR213, PR216 | 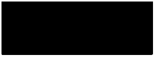 | | |
| SEQ ID NO: 29 and SEQ ID NO: 30SEQ ID NO: 33 and SEQ ID NO: 34SEQ ID NO: 37 and SEQ ID NO: 38SEQ ID NO: 40 and SEQ ID NO: 41SEQ ID NO: 43 and SEQ ID NO: 44 | PR217, PR218PR219, PR220PR221, PR222PR256, PR257PR253, PR254 | SEQ ID NO: 26 (443 bp)SEQ ID NO: 31 (506 bp)SEQ ID NO: 35 (2279 bp) | 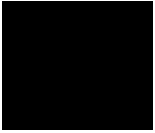 | SEQ ID NO: 28 (440 bp) |
| SEQ ID NO: 47 and SEQ ID NO: 48 | PR245, PR247 | SEQ ID NO: 45 (4027 bp) | SEQ ID NO: 45 (4027 bp) | 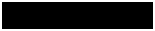 |

In some embodiments, at least one primer of a primer pair binds specifically to a polynucleotide that, is present in P. acnes Group 2 or 3. Those skilled in the art could identify primers that would identify such sequence.

In a fifth aspect, the present invention provides a probe specific for P. acnes, wherein the probe detects or localizes a P. acnes nucleic acid or antigen.

P. acnes probes, includes polynucleotides, oligonucleotides, and fragments thereof, PCR primers and antibodies, domain antibodies or fragments thereof against a P. acnes antigen, that can be used to detect or identify or image localize P. acnes in a subject or a sample, for example, for the purpose of detecting or diagnosing a prostate disease or condition. The probe is preferably specific for P. acnes associated with a prostate disease. The probe is preferably specific for all P. acnes or for specific subgroups. The probes may be linked to a label (such as biotin, radioisotopes, paramagnetic metals, fluorescent molecules, and chemiluminescent moieties) or linked or attached to a support (e.g., beads, particles, dipsticks, fibers, membranes, and silane or silicate supports such as glass slides).

In a preferred embodiment, the P. acnes specific probes such as polynucleotides, oligonucleotide, primers, antibodies described herein further comprises a detectable label, is attached to a solid support, is prepared at least in part by chemical synthesis, is single stranded (for polynucleotides or primers), is double stranded (for polynucleotides or primers), or is part of a micro-array.

Polynucleotides described herein comprising a sequence specific for A acnes and P. acnes specific subgroups, or hybridizing to DNA from P. acnes Group 1 and/or Group 2 and/or Group 3 under high stringency, may be used as polynucleotide probes.

In a further preferred embodiment, the invention provides antibodies to P. acnes and P. acnes specific subgroups 1, 2 and 3. In some embodiments, the antibody specifically binds to an antigen associated with all P. acnes. In some embodiments, the antibody specifically binds to an antigen associated with Group 1 and/or Group 2 and/or Group 3 P. acnes. The antibodies can be polyclonal or monoclonal, or made by molecular biology techniques, and can be labeled with a variety of detectable labels, including but not limited to radioisotopes, paramagnetic metals, fluorescent molecules, and chemiluminescent moieties.

“Oligonucleotide,” as used herein, generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab*)₂, Fv), single chain (ScFv), mutants thereof, fusion proteins comprising an antibody portion, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by recombinant DNA methods such as described in U.S. Pat. No. 4,816,567. The monoclonal antibodies may also be isolated from phage libraries generated using the techniques described in McCafferty et al., 1990, Nature, 348:552-554, for example.

Methods of making polyclonal and monoclonal antibodies are known in the art. One method which may be employed for making monoclonal antibodies is the method of Kohler and Milstein (1975) or a modification thereof. In general, a mouse or rat is used for immunization but other animals may also be used. The immunogen can be, but is not limited to, P. acnes cells (preferably P. acnes Group 1 and/or Group 2 and/or Group 3), components of the cells, proteins, polypeptides, polynucleotides, lipids, carbohydrates. Bacterial cells or other immunogens may be used in combination with anon-denaturing adjuvant or a denaturing adjuvant, such as Ribi and Freud' adjuvant. The immunogen may be administered multiple times at periodic intervals such as, bi-weekly, or weekly, or may be administered in such a way as to maintain viability in the animal (e.g., in a tissue recombinant).

To monitor the antibody response, a small biological sample (e.g., blood) may be obtained from the animal and tested for antibody titer against the immunogen. The spleen and/or several large lymph nodes can be removed and dissociated into single cells. If desired, the spleen cells may be screened (after removal of non-specifically adherent cells) by applying a cell suspension to a plate or to a well coated with the antigen. B-cells, expressing membrane-bound immunoglobulin specific for the antigen, will bind to the plate, and are not rinsed away with the rest of the suspension. Resulting B-cells, or all dissociated spleen cells, can then be fused with myeloma cells (e.g., X63-Ag8.653 and those from the Salk Institute, Cell Distribution Center, San Diego, Calif.). Polyethylene glycol (PEG) may be used to fuse spleen or lymphocytes with myeloma cells to form a hybridoma. The hybridoma is then cultured in a selective medium (e.g., hypoxanthine, aminopterin, thymidine medium, otherwise known as “HAT medium”). The resulting hybridomas are then plated by limiting dilution, and are assayed for the production of antibodies which bind specifically to the immunogen using FACS, immunohistochemistry, Western blot, or any other immunoassays. The selected monoclonal antibody-secreting hybridomas are then cultured either in vitro (e.g., in tissue culture bottles or hollow fiber reactors), or in vivo (e.g., as ascites in mice). Monoclonal antibody-secreting hybridomas described above can be further selected for producing antibodies that bind preferentially to antigens from P. acnes Group 1 and/or Group 2 and/or Group 3.

As another alternative to the cell fusion technique, EBV immortalized B cells may be used to produce monoclonal antibodies of the subject invention. The hybridomas are expanded and subcloned, if desired, and supernatants are assayed for anti-immunogen activity by conventional assay procedures (e.g., FACS, IHC, radioimmunoassay, enzyme immunoassay, fluorescence immunoassay, etc.).

In another alternative, the antibodies can be made recombinantly. Methods for making recombinant antibodies are well-known in the art. Monoclonal antibodies selected can be sequenced and produced (including various formulations of antibodies, such as antibody fragments, scFv, and fusion proteins) recombinantly in vitro.

The term “label” refers to a composition capable of producing a delectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition delectable by spectroscopic, photochemical, biochemical, immunochemical, electrical, optical, chemical, or any other appropriate, means. The term “label” is used to refer to any chemical group or moiety having a detectable physical property or any compound capable of causing a chemical group or moiety to exhibit a detectable physical property, such as an enzyme that catalyses conversion of a substrate into a detectable product. The term “label” also encompasses compounds that inhibit the expression of a particular physical properly. The label may also be a compound that is a member of a binding pair, the other member of which bears a detectable physical property.

An alternative method involves detecting the presence of an antigen of P. acnes, preferably Group 2 and/or Group 3 using the nanoparticle-based bio-bar codes method.

Nam (2003). This method relies on magnetic microparticle probes with antibodies that specifically bind a target protein and nanoparticle probes that are encoded with DNA (complementary to bar-code) that is unique to the target protein and antibodies that can sandwich the target protein captured by the microparticle probes. Magnetic separation of the completed probes and target followed by dehybridization of the oligonucleoties (bar-code) on the nanoparticle probe surface allows the determination of the presence of the target protein by identifying the bar-code released from the nanoparticle probe. Because the nanoparticle probe carries with it a large number of oligonucleoties per antibody, there is substantial amplification, which increases sensitivity of the method.

A further preferred embodiment involves analysis of the sample for the presence of metabolic products of P. acnes by methods appropriate for the product itself.

In a sixth aspect the present invention provides a kit for diagnosing the presence of, or the predisposition to develop, prostate disease in a subject, the kit comprising at least one P. acnes specific probe. In a preferred embodiment, the probe is specific for P. acnes Group 1 and/or Group 2 and/or 3. Preferably, the probe is specific for P. acnes that comprise the DNA sequence of SEQ ID NO:1 or SEQ ID NO:2. More preferably, the probe is specific for P. acnes Group 2 and/or Group 3 as herein defined. The probe includes primers (e.g., PCR primers), other polynucleotides, and/or antibodies described herein.

The kits may be in any suitable packaging, and may optionally provide additional components such as, buffers and instructions for using the P. acnes specific probe in any of the diagnosing methods described herein.

In a seventh aspect, the present invention also provides methods of screening for an agent that has inhibitory effect on P. acnes, wherein the methods comprise incubating P. acnes in the presence of an agent and detecting inhibitory effect of the agent on P. acnes. Preferably, the inhibitory effect is specific for P. acnes and has no effect to a subject (such as human). The methods may further comprise a step of comparing the inhibitory effect of the agent on P. acnes Group 1 and/or Group 2 and/or Group 3, and selecting the agent that selectively inhibits P. acnes Group 1 and/or Group 2 and/or Group 3. The screening test may be performed in conjunction with high throughput screening techniques to allow screening multiple agents and multiple strains of P. acnes at the same time.

Any component(s) of P. acnes, such as target regulatory system and biological pathways may also be used for inhibitory agent screening.

Any agent may be screened for inhibitory effect to P. acnes. Such agents may be any molecules including organic or inorganic molecules, e.g., protein, oligopeptide, small organic or inorganic molecule, polysaccharide, polynucleotide, fatty acids, steroids, purines, pyrimidines, derivatives, structural analogs, antibiotics, or combinations thereof. Agents may also be obtained from a wide variety of sources including libraries of synthetic or natural compounds. Additionally, natural or synthetically produced libraries and compounds are readily modified through conventional chemical, physical and biochemical means. Known pharmacological agents (e.g., known antibiotics) may be subjected to directed or random chemical modifications, such as acylation, alkylation, esterification, or amidification to produce structural analogs.

In an eighth aspect of the present invention there is provided a method of preventing or treating a prostate disease in a subject, the method comprising administering to a subject in need thereof an effective amount of a P. acnes inhibitory composition.

Preferably, the P. acnes inhibitory composition comprises at least one antibiotic. In an alternative embodiment the composition is a prophylactic vaccine comprising at least one P. acnes antigen. In a preferred embodiment the antigen is derived from P. acnes Group 1 and/or Group 2 and/or Group 3.

The prostate disease is preferably selected from the group consisting of prostatitis, dysplasia (pre-cancer) and prostate cancer.

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, decreasing the dose of other medications required to treat the disease, and remission (whether partial or total), whether detectable or undetectable. “Treatment” can also mean prolonging survival as compared to expected survival if not receiving treatment. “Treatment” is an intervention performed with the intention of preventing the development or altering the pathology of a disorder. Accordingly, “treatment” refers to both therapeutic treatment and prophylactic or preventative measures.

The term “effective amount” means a dosage sufficient to provide prevention of the prostate disease or effective beneficial or desired clinical results. This will vary depending on the subject and the disease/condition being affected. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

A “vaccine” is a pharmaceutical composition for human or animal use, particularly an immunogenic composition which is administered with the intention of conferring the recipient with a degree of specific immunological reactivity against a particular target, or group of targets (i.e., elicit and/or enhance an immune response against a particular target or group of targets). The immunological reactivity, or response, may be antibodies or cells (particularly B cells, plasma cells, T helper cells, and cytotoxic T lymphocytes, and then precursors) that are immunologically reactive against the target, or any combination thereof. For purposes of this invention, the target is primarily P. acnes or an antigen from P. acnes.

Vaccines may be subunit vaccines or whole organism vaccines. Subunit vaccines are prepared from components of the whole organism and are usually developed in order to avoid die use of live organisms that may cause disease or to avoid the toxic components present in whole organism vaccines. See, e.g., Parke (1977), Anderson (1977); and Makela (1977). Subunit vaccines can be prepared by chemical inactivation of partially purified toxins. Formaldehyde or glutaraldehyde have been the chemicals of choice to detoxify bacterial toxins. Whole organism vaccines make use of the entire organism for vaccination. The organism (e.g., P. acnes Group 1 and/or 2 and/or 3) may be killed or alive (usually attenuated) depending upon the requirements to elicit protective immunity. Methods of generating killed or live but attenuated vaccines are known in the art. Sec, e.g., U.S. Pat. No. 4,016,253; Brown (1959); U.S. Pat. No. 5,294,441; U.S. Pat. No. 5,210,035; PCT WO 00/45840.

In a ninth aspect of the present invention there is provided an isolated subtype of P. acnes Group 2 or Group 3 as hereinafter defined.

The following discussion provides characteristic of P. acnes groups 1, 2 and 3:

P. acnes Group 3 comprise polynucleotide sequence MMCoA sequence B (SEQ ID NO:1) whereas Groups 1 and 2 comprise MMCoA sequence A (SEQ ID NO:2).

As used herein “P. acnes Group 1” are distinguished by having at least the following identifying characteristics:
1. comprising polynucleotide sequence MMCoA sequence A (SEQ ID NO:2) for Group 1.
2. lack of DNA sequence as specified in SEQ ID NO:6.
3. comprising a DNA sequence of about 8.7 kb which encodes the following open reading frames: N-acetyl-beta-hexosamimidasc; ABC peptide transporter, permease component 1; ABC peptide transporter, permease component 2; ABC peptide transporter, ATP-binding component 1; ABC peptide transporter, ATP-binding component 2; ABC peptide transporter, solute-binding protein; and Chitinase as specified in SEQ ID NO:10;
4. lack of the polynucleotide sequence as specified in SEQ ID NO:11; and
5. comprising a polynucleotide sequence SEQ ID NO:15
6. comprising a polynucleotide sequence SEQ ID NO:20
7. comprising a polynucleotide sequence SEQ ID NO:23
8. comprising a polynucleotide sequence SEQ ID NO:26
9. comprising a polynucleotide sequence SEQ ID NO:31
10. comprising a polynucleotide sequence SEQ ID NO:35
11. comprising a polynucleotide sequence SEQ ID NO:45

The present invention also provides an isolated subtype of P. acnes Group 2 or Group 3, as hereinafter defined. As used herein “P. acnes Groups 2” and “P. acnes Group 3” are distinguished by having at least the following identifying characteristics:
1. a hydrophilic surface as determined using SAT tests and liquid growth characteristics;
2. comprising polynucleotide sequence MMCoA sequence B (SEQ ID NO:1) for Group 3 and MMCoA sequence A (SEQ ID NO:2) for Group 2.
3. comprising polynucleotide sequence of SEQ ID NO:6.
4. lack of a DNA sequence of about 8.7 kb which encodes the following open reading frames: N-acetyl-beta-hexosaminidase; ABC peptide transporter, permease component 1; ABC peptide transporter, permease component 2; ABC peptide transporter, ATP-binding component 1; ABC peptide transporter, ATP-binding component 2; ABC peptide transporter, solute-binding protein; and Chitinase as specified in SEQ ID NO: 10;
5. comprising polynucleotide sequence of SEQ ID NO:11; and

P. acnes Group 2 also has the following additional characteristics:
1. comprising polynucleotide sequence of SEQ ID NO: 16; and
2. comprising polynucleotide sequence of SEQ ID NO:27.
3. comprising polynucleotide sequence of SEQ ID NO:32.
4. comprising polynucleotide sequence of SEQ ID NO:36.
5. comprising polynucleotide sequence of SEQ ID NO:39.
6. comprising polynucleotide sequence of SEQ ID NO:42.
7. comprising polynucleotide sequence of SEQ ID NO:45.

P. acnes Group 3 also has the following additional characteristics:
1. comprising polynucleotide sequence of SEQ ID NO:17; and
2. comprising polynucleotide sequence of SEQ ID NO:28.
3. comprising polynucleotide sequence of SEQ ID NO:46.

The presence of P. acnes Group 2 and/or Group 3 may be identified using any methods described herein.

A type strain of P. acnes Group 3 has been deposited under the Budapest Treaty with Australian Government Analytical Laboratories (AGAL) on Nov. 2, 2004 and has been accorded Accession No. NM04/39927. A type strain of P. acnes Group 2 has also been deposited under the Budapest Treaty with Australian Government Analytical Laboratories (AGAL) on Sep. 6, 2004 and has been accorded Accession No. NM04/41610. These may be contrasted against the P. acnes strain deposited as ATCC 6919, which is a Group 1 P. acnes.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments. It should be understood that the invention as claimed should not be unduly limited to such specific embodiments. Indeed, various modifications of the described modes for carrying out the invention, which are apparent to those skilled in molecular biology or related fields, are intended to be within the scope of the invention.

Any discussion of documents, acts, materials, devices, articles or the like which has been included in the present specification is solely for the purpose of providing a context for the present invention. It is not to be taken as an admission that any or all of these matters form part of the prior art base or were common general knowledge in Australia in the field relevant to the present invention

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In order that the nature of the present invention may be more clearly understood preferred forms thereof will now be described by reference to the following non-limiting Examples.

### EXAMPLES

### Example 1

### Assessment of Prostate Tissue from Prostate Cancer Patients Methods

### 1. Prostate Tissue Samples

Samples of prostate tissue were collected from radical prostatectomy specimens of prostate cancer patients undergoing curative intent surgery. All patients were identified with an elevated scrum PSA (>4 ng/ml) and the diagnosis was confirmed on pre-operative needle biopsy. No patient had a history or clinical symptoms of bacterial prostatitis and none were in acute urinary retention. Immediately following prostectomy, the posterolateral aspect of both prostatic lobes were incised superficially to avoid entry into the ejaculatory ducts, transition zone or prostatic urethra and triplicate samples of 100-200 mg of macroscopically normal tissue were taken under sterile conditions within 30 minutes of resection. One sample was stored at −80° C. for DNA extraction while two samples were finely macerated under sterile conditions for duplicate bacterial culture.

2. Culture, and Identification of Bacteria from Prostate Tissue

Tissue specimens were incubated without agitation at 37° C. for up to 30 days in brain heart infusion (BHI) broth (Oxoid Australia Ltd, West Heidelberg, Victoria) supplemented with 5% horse scrum. Positive broth cultures were subcultured onto BHI agar (Oxoid Australia Ltd) with 5% horse serum at 37° C. in an atmosphere generation jar with CO₂ enrichment. Micro-organisms were identified by morphological characteristics and sequencing of the 16S rRNA gene as follows. Bacterial pellets were resuspended in 128 μL of sterile phosphate-buffered saline (PBS) and bacterial cell walls disrupted using a Mixer Mill (MM301, Reicht, Germany). This involved addition of acid-washed 0.1 mm silica/zirconia beads (Biospec Products Inc, USA) and bead-beating for 2 mins at 30 hz, followed by extraction with the QiaAmp DNA mini kit (Qiagen Pty Ltd, Clifton Hill, Australia) using the tissue protocol according to the manufacturer's instructions. PCR was performed with primers 16S1F, 16S1R, 16S2F and 16S2R (Table 2), which amplify the bacterial 16S rRNA gene in two segments of 801 bp and 875 bp respectively (modified from Relman 1992). Thermal cycling conditions for both primer pairs were 10 mins at 95° C., 35 cycles of 30 seconds at 95° C., 30 seconds at 59° C. and 1 min at 72° C., followed by 7 min extension at 72° C. Deep Vent DNA Polymerase (New England Biolabs, Beverly, Me.) was used for PCR reactions involving universal bacterial primers because it contains little or no endogenous bacterial DNA. PCR products were sequenced and compared with, those in Genbank using the BLAST function.

TABLE 2

Primers used for sequencing of the bacterial 16S
rRNA gene from isolated organisms.

| | | |
|---|---|---|
| Primer | Sequence (5′-3′) | SEQ. ID NO. |

| | | |
|---|---|---|
| 16S1F | TGAAGAGTTTGATCCTGGCTCAG | SEQ. ID NO:49 |
| 16S1R | GGACTACCAGGGTATCTAAKCCTG | SEQ. ID NO:50 |
| 16S2F | GTGCCAGCAGCCGCGGTRA | SEQ. ID NO:51 |
| 16S2R | AGSCCCGGGAACGTATTCAC | SEQ. ID NO:52 |

A = Adenosine, G = Guanosine, C = Cytidine, T = Thymidine, K = G or T, R = A or G and S = C or G

3. Association of Inflammation with the Isolation of Bacteria from the Prostate

Whole mount hematoxylin and eosin stained tissue sections from each case were used to assess and quantitate the inflammation in the peripheral zone (Table 3).

Associations between the presence of inflammation and culture for P. acnes were determined using Fisher's exact lest. The unpaired t-test was used to compare the extent of inflammation between patient groups positive or negative for culture of P. acnes. In all cases a two-tailed p value of < 0.05 was taken to indicate statistical significance.

TABLE 3
Criteria used to grade the degree of prostatic inflammation

| | |
|---|---|
| Acute | |
| Inflammation: | |
| Grade 1 | Isolated polymorphonuelear neutrophils (PMN) in |
| | lining epithelium and clusters of PMN in gland lumens |
| Grade 2 | Large numbers of PMN migrating through gland walls |
| | with epithelial disruption |
| Grade 3 | Micro abscesses and necrosis |
| Chronic | |
| Inflammation: | |
| Grade 1 | Isolated lymphohistiocytic aggregates in the stroma |
| | surrounding glands |
| Grade 2 | Lymphoid follicles, displacement of glands by |
| | inflammatory aggregates. |
| Grade 2 | Lymphoid follicles, displacement of glands by |
| | inflammatory aggregates. |
| Grade 3 | Granulomatous prostatitis. |

### Results

1. Bacteria Isolated by Culture from Prostate Tissue

Positive bacterial cultures were obtained from 19/34 (56%) prostate cancer patients. The predominant micro-organism was Propionibacterium acnes (P. acnes), found in 12 (35%) cases. Less frequent isolates included various species of coagulase-negative staphylococcus, with only single isolates of bacillus, lactobacillus and corynebacierium species (Table 4). Staphylococcus and bacillus cultures became turbid within 24-48 hours whereas most P. acnes cultures took 8-15 days of incubation to show visible growth. This suggests that any P. acnes organisms present in mixed culture with fast-growing bacteria would have been overgrown and likely missed. Sub-cultures of P. acnes took 24-48 hours to produce visible growth confirming both a slow growing organism as well as very scant organism numbers in the primary culture.

TABLE 4
Bacteria isolated from prostatic tissue of
34 unselected prostate cancer patients

| Organism | No Patients* (%) |
|---|---|
| No bacterial growth | 15 (44) |
| Propionibacterium acnes | 12 (35) |
| Staphylococcus epidermidis | 6 (18) |
| Staphylococcus warneri | 1 (3) |
| Staphylococcus saccharolyticus | 1 (3) |
| Lactobacillus iners | 1 (3) |
| Corynebacterium sp. | 1 (3) |
| Bacillus cereus | 1 (3) |
| Bacillus subtilis | 1 (3) |

*4 patients grew P. acnes and a different organism from the duplicate samples and one patient cultured P. acnes in both samples. A further patient grew both P. acnes and a slow growing Corynebacterium from the same sample

Therefore, in this study low-virulent Gram-positive bacteria were cultured from prostatic tissue of 19/34 (56%) patients with prostate cancer. The predominant micro-organism was P. acnes, cultured from 12 (35%) different patients, followed by S. epidermidis from 6 (18%). These results differ significantly from the few previous studies involving isolation and identification of bacteria from prostatic tissue of cancer patients. An infectious aetiology for prostate cancer was originally investigated in the early 1900's (Dudgeon 1904, Rosen 1918) when culture of open prostatectomy samples yielded, mainly staphylococcus and Escherichia colli (15-54% and 18-26% of cases respectively). Studies in the late 1980's involved prostate tissue from transurethral resections (TUR), with aerobic culture (Gorelick 1988) isolating mainly E. coli (44%), Streptococcus faecalis (23%) and Staphylococcus epidermidis (18%) while anaerobic culture techniques (Cooper 1988) yielded anaerobic cocci (44%), Bacteroides distasunis and Clostridium perfringens (11% each). Although the finding of S. epidermidis in 18% of prostatectomy specimens correlates with the incidence reported in the above studies, the results differ in that i) aerobic colonic micro-organisms such as E. coli or S. faecalis were not isolated, and ii) the predominant isolate was P. acnes, a bacterium not previously cultured from prostate cancer patients.

These two differences may be due to the particular patient population and culture techniques presently used. In the previous studies most tumors were incidentally discovered during prostate resection (Dudgeon 1904, Rosen 1918) or TUR (Gorelick 1988, Cooper 1988) for obstructive symptoms and in some cases the patients had current urinary tract infections frequently involving intestinal bacteria such as E. coli (Dudgeon 1904, Rosen 1918, Gorelick 1988). In contrast, patients in the present study were undergoing prostatectomy for carcinoma diagnosed by rising PSA levels and subsequent carcinoma positive biopsy, none were in urinary retention or had a current urinary tract infection. Secondly, culture conditions in the previous studies were not optimal for detection of P. acnes because they used either aerobic culture on blood agar (Gorelick 1988 and Dudgeon 1904) or an incubation time of only seven days for anaerobic culture (Cooper 1988). P. acnes is a microaerophilic bacterium better suited to low oxygen levels (Webster 1995) and most prostate specimens were found to require an incubation time of 8-15 days to obtain visible growth of this organism. A similar incubation lime of up to 10 days was required to obtain visible growth of P. acnes from vitreous samples of patients with chronic infectious endopthalmitis (Hall 1994).

Although P. acnes has not previously been isolated from prostate cancer patients, it has been cultured from urethral swabs and prostate biopsies from approximately 2% of healthy males (Willen 1996, Lee 2003), indicating that this bacterium can colonize the male genital tract. Cultures of prostate biopsies from men with chronic prostatitis have shown a slightly increased detection rate of propionibacterium (not identified to species level), found in 3.5% to 6% of patients (Berger 1997, Lee 2003). However propionibacteria were not considered pathogenic in these studies because other bacteria were more frequently isolated, including Gram-negative rods (20%), coagulase-negative staphylococci (11-15%) and aerobic diptheroids (16-20%). The aerobic diptheroids from prostatitis cases were identified as corynebacteria in a separate study by Tanner et al (1999). The present results show a considerably higher incidence of P. acnes in prostatic tissue from prostate cancer patients compared to the incidences previously detected in healthy males or men with prostatitis.

### 2. Association Between Bacterial Culture and Prostatic Histology

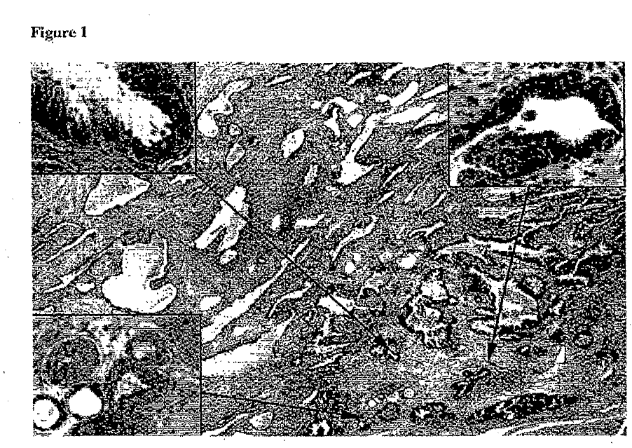

No correlation was observed between bacterial culture results and clinicopathological factors such as patient age at diagnosis, tumour site of origin (transition versus peripheral zone), cancer multifocality, tumour grade or pathological stage (data not shown). Foci of acute inflammation (grades 1-3) were observed in 15/34 (44%) radical prostatectomy specimens while focal chronic inflammation occurred in 23/34 (68%) specimens (FIG. 1). Analysis of inflammatory foci by tissue Gram stain did not detect bacteria in any case, however statistically significant associations were observed between positive culture for P. acnes and both the presence and the extent of acute and chronic inflammation in the radical prostatectomy specimen (Table 5). Specimens with a positive culture for bacteria other than P. acnes showed slightly increased inflammation but this trend was entirely due to two cases, which grew B. cereus and S. saccharolyticus respectively. Cases positive for culture of S. epidermidis, S. warneri, B. subtilis, L. iners or Corynebacterium sp. showed no evidence of acute inflammation and no increase in degree of chronic inflammation compared to negative controls.

Our repeated failure to identify P. acnes or any other organism on direct tissue gram stain is confusing, but has also been noted by other authors investigating culture-positive P. acnes infections (Esteban 1996, Stirling 2001). Failure to directly detect P. acnes in tissue sections almost certainly reflects the low numbers of infecting bacteria but may also be in part due to poor uptake of the Gram stain by these micro-organisms in vivo as a result of changes to the bacterial wall induced by the immune response (Esteban 1996). It is also possible that these organisms only populate the prostatic secretions with minimal or no direct tissue invasion and thus may be flushed from tissue samples during the fixation and tissue processing. The finding of a significant positive association between culture of P. acnes and inflammation in prostatectomy specimens does however provide an indirect link between this bacterium and prostatic inflammation. P. acnes is known to be a potent stimulus to the lympho-reticular system, capable of producing an inflammatory response without direct tissue invasion by secretion of soluble irritant agents that diffuse into tissues and attract human neutrophils (Webster 1995). P. acnes is also highly resistant to killing and degradation by human neutrophils and monocytes (Webster 1995), a characteristic that allows it to establish long-term low-grade infections that may persist for decades (Sabel 1999). It has recently been linked to several other chronic inflammatory conditions including sarcoidosis (Yamada 2002) and sciatica (Stirling 2001).

TABLE 5
Association between bacterial culture results and
inflammation in radical prostatectomy specimens.

| | No Bacterial | ¹Other Bacteria | P. acnes |
|---|---|---|---|
| | Growth | Cultured | Cultured |
| Parameter | (n = 15) | (n = 7) | (n = 12) |
| Acute inflammation | 3 (20%) | 2 (29%) | 9 (75%) |
| present | | P = 1.0 | ²P = 0.007 |
| (Grade 1, 2 or 3) | | | |
| Extent of acute | 2.7 | 3.8 | 16.7 |
| inflammation | | ³P = 0.7 | P = 0.007 |
| (mean % of glands | | | |
| involved) | | | |
| Chronic inflammation | 6 (40%) | 5 (71%) | 11 (92%) |
| present | | P = 0.4 | P = 0.01 |
| ⁴(Grade 1, 2 or 3) | | | |
| Extent of chronic | 5.3 | 7.5 | 14.2 |
| inflammation (mean | | P = 0.5 | P = 0.01 |
| % of glands involved) | | | |

¹These include only cases negative for growth of P. acnes in both duplicate culture samples.
²P values for comparing the presence of inflammation between groups were obtained by Fisher's Exact Test. Each group is compared back to the group negative for bacterial culture.
³P values for comparing the extent of inflammation between groups were obtained by unpaired t-test. Each group is compared back to the group negative for bacterial culture.
⁴No cases of chronic inflammation grade 3 (granulomatous prostatitis) were observed.

### Example 2

### Characterization and Comparison of Cultured P. acnes Isolated from Prostate and Facial Skin

### Methods

1. Isolation of Cutaneous P. acnes for Comparison

Multiple isolates of P. acnes were obtained from the facial skin of two healthy male volunteers using the swabbing technique described by McGinley (1978), using limiting dilution to achieve single colonies. These cutaneous isolates were initially grown on BHI agar plates in an atmosphere generation jar with CO₂ enrichment, then sub-cultured into BHI broth and identified by DNA sequencing with 16S primers as described in Example 1 for prostatic isolates.

### 2. Growth Characteristics

When grown in culture without any agitation it was observed that cultures of some P. acnes isolates maintained a suspension within the liquid media regardless of the density, while others remained in suspension only at low density and upon reaching a particular density formed aggregates and settled as a sediment on the bottom of the culture bottle, leaving a clear supernatant. This observation related to the cell surface hydrophobicity as described below,

### 3. Salt Aggregation Tests (SAT)

SATs to determine bacterial cell surface hydrophobicity were carried out as described by Jonsson and Wadstrom (1984). 1.5 mL of stationary phase P. acnes broth cultures were resuspended in 700 μL of 0.02M sodium phosphate buffer (pH 6.8) and 25 μL aliquots were mixed on slides for 2 min with equal volumes of ammonium acetate at various concentrations (0.5-8.0 M). The lowest concentration of salt giving visible bacterial clumping was taken as the SAT score. An aliquot of bacterial cells without added salt were used as a negative control to test for auto-aggregation.

### 4. Pulsed-Field DNA Analysis

Genomic DNA from P. acnes isolates (prostatic and cutaneous) was analyzed by the method described in Ting et al (1999). DNA was run on a FIGE Mapper field inversion system (Biorad MA, USA) on a 1% pulse field agarose gel in 0.45×TBE with a forward and reverse voltages of 180 and 120 volts with 0.1 to 2 sec linear switch times for 16 hrs.

### 5. MMCoA Gene Sequence Analysis

The primers MMF (SEQ ID NO:3) and MMR (SEQ ID NO:4) were designed in our laboratory to specifically amplify a 633 bp region of the P. acnes “methylmalonyl-CoA carboxyltransferase subunit 12S monomer” gene, which encodes the 12S subunit of the transcarboxylase enzyme that catalyses production of propionate (Thornton 1993). PCR specificity tests confirmed that primers SEQ ID NO:3 and SEQ ID NO:4 do not show any cross-reactivity with i) human DNA, ii) the endogenous bacterial DNA in HotStarTaq DNA polymerase, iii) a panel of Gram positive bacilli including Propionibacterium granulosum plus corynebacterium, actinomyces and bacillus species, or iii) DNA from any of the bacterial species isolated from prostatic tissues in this study except for P. acnes. These primers do however amplify the correct 633 bp region of the MMCoA gene from Propionibacterium avidum, which is considered to be the closest genetic relative of P. acnes. The DNA sequence of the PCR products from P. acnes and P. avidum differ considerably and can therefore be distinguished by sequencing. PCR cycling conditions were 15 mins at 95° C., 40 cycles of 30 seconds at 94° C., 1 min at 55° C. and 1 min at 72° C., followed by 7 min extension at 72° C. HotStarTaq DNA Polymerase (Qiagen Pty Ljtd) was used for this PCR reaction. The amplified PCR products were sequenced and the DNA sequences of the various P. acnes isolates were aligned and compared using the Clustal W program.

### Results

To determine whether P. acnes isolated from the prostate differ from those colonizing normal human skin, six P. acnes isolates were obtained from the skin of two healthy male volunteers as described above. Prostate and cutaneous isolates were characterized and compared as follows:

### 1. Growth Characteristics and Cell Surface Properties

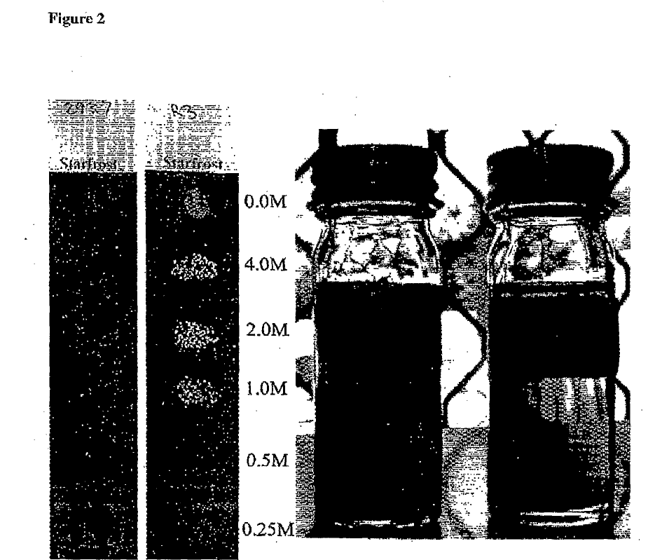

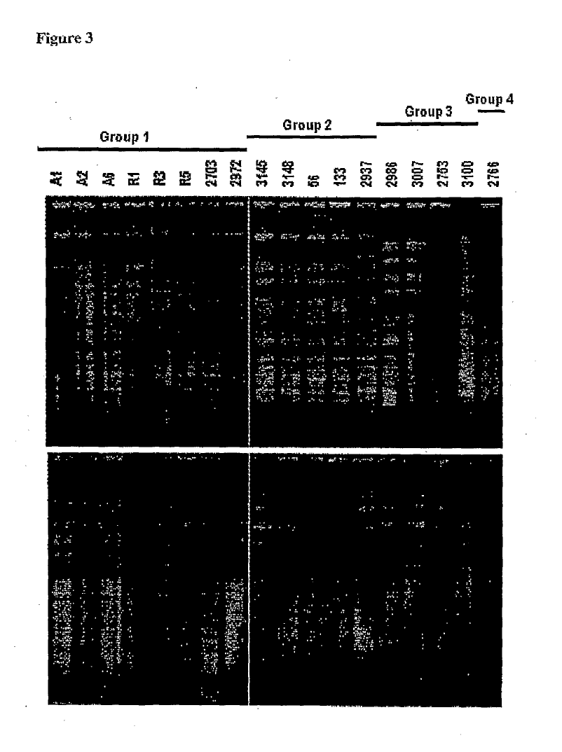

All of the 6 skin P. acnes isolates grew as a granular sediment with clear supernatant when cultured in liquid medium without agitation (FIG. 2). This growth characteristic was also observed for 2 of the 12 prostate isolates of P. acnes. In contrast the remaining 10 P. acnes isolates from the prostates grew as fine sediment with a turbid supernatant shown by Gram staining to contain suspended cells. These observations were mirrored in the analysis of cell surface properties using SAT tests where the 6 skin isolates and the same two prostate isolates were hydrophobic, showing complete aggregation in 1M salt, whereas the remaining 10 prostate isolates were hydrophilic, showing only a small degree of aggregation in 2-4M salt (FIG. 2).

### 2. Genomic DNA Analysis by Pulse Field Electrophoresis

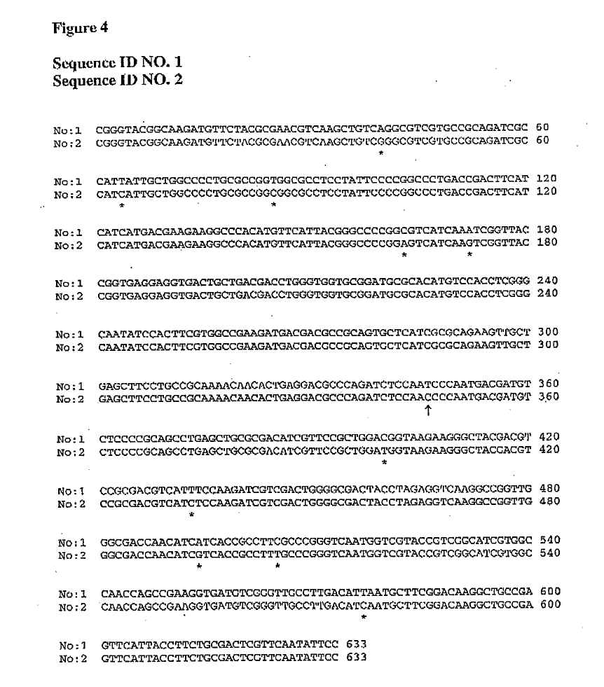

Genomic DNA extracted from the bacterial cultures was compared by pulsed-field gel DNA electrophoresis. Analysis of the DNA banding patterns revealed three main groups of P. acnes (FIG. 3). Group 1 consisted of all six cutaneous isolates and two prostatic isolates—the same two that demonstrated sedimentary growth characteristics and a hydrophobic cell surface. Nine of the remaining prostatic P. acnes could be divided into Group 2 (5 isolates) and Group 3 (4 isolates), whereas one isolate (02-2766) differed from all others and was assigned into Group 4. The phenotypic growth characteristics were reflected in the genomic banding comparisons and these results suggest that most P. acnes cultured from the prostate are genetically and phenotypically distinct from those colonizing human skin.

### 3. MMCoA Gene Sequence Analysis of Isolated Organisms

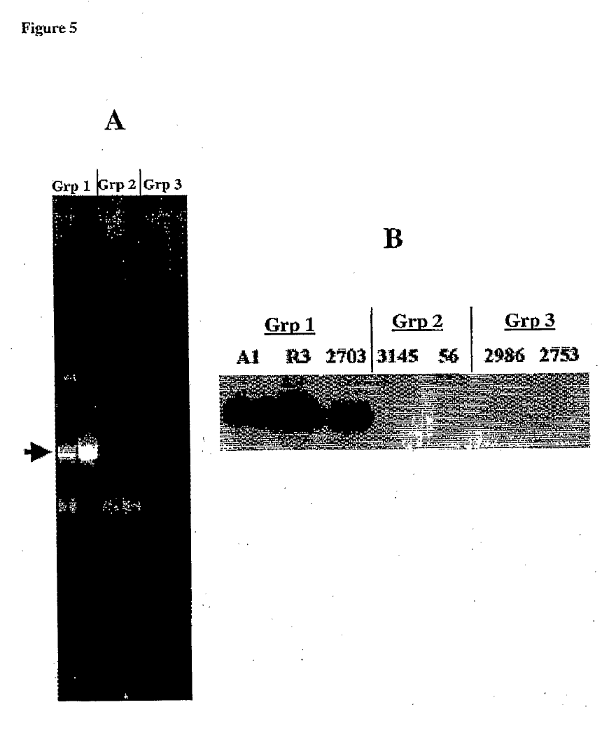

Analysis of MMCoA gene sequences revealed two distinct sequence types designated SEQ ID NO:1 and SEQ ID NO:2 that differ at 10 specific single-base positions (and one position that is polymorphic) within the 633 bp region characterized by sequencing of the PCR product amplified by the MMCoA primers MMF (SEQ ID NO:3) and MMR (SEQ ID NO:4) (FIG. 4). AU P. acnes from pulsed-field Groups 1 and 2 were found to have SEQ ID NO:2, whereas P. acnes from Group 3 had SEQ ID NO:1. Prostatic isolate 02-2766 (Group 4) had an MMCoA sequence intermediate between SEQ ID NO:1 and 2 with several additional single-base differences.

Taken together, these results indicate that the P. acnes isolates fall into three main groups:

1) Group 1 (mainly cutaneous isolates) have SEQ ID NO:2 and a hydrophobic cell surface;
2) Group 2 (prostatic isolates) have SEQ ID NO:2 and a hydrophilic cell surface; and
3) Group 3 (prostatic isolates) have SEQ ID NO:1 and a hydrophilic cell surface.

This classification correlates with genetic groupings obtained by pulsed-field gel DNA analysis. The finding of a single isolate that differed by both pulsed-field analysis and MMCoA sequence suggests that other less common groups may also exist.

Therefore, the present results show that particular sub-types of P. acnes occur in prostatic tissue from prostate cancer patients. These prostatic sub-types (Pulse-field Groups 2, 3 and 4) differ from common cutaneous isolates genetically, as shown by pulsed-field gel DNA analysis, and phenotypically, in having hydrophilic cell surface properties. It is therefore interesting to note that a hydrophilic cell surface has been associated with resistance to phagocytosis and increased virulence in several bacterial species (Van Oss 1978). It is possible that MMCoA SEQ ID NO:1 is associated with P. acnes of serotype n, since 85% of the isolates from Groups 1 and 2 fermented sorbitol (indicative of serotype I) (Kishishita 1979, Sasaki 1980a) whereas all of the Group 3 isolates were negative for sorbitol fermentation (data not shown).

In conclusion, P. acnes was identified as the predominant micro-organism in the prostate gland of patients with localized prostatic carcinoma. Assessment of prostate tissues at an earlier stage, prior to development of post-inflammatory atrophy, dysplasia and cancer, may reveal increased organism numbers. A therapeutic antibiotic regime targeted to P. acnes could alter the natural development of this common tumor.

### Example 3

### Isolation of DNA Sequences which Differ Between P. acnes Groups 1, 2 and 3 to Allow Design of Primers Specific for Each Group

### Methods

### 1. Use of RAPD-PCR to Identify DNA Sequence Variations

DNA sequence variations between P. acnes isolates of Groups 1, 2 and 3 wore sought using the standard PCR method of Random Amplified Polymorphic DNA (RAPD-PCR) as previously described (Rossi et al, 1998). This involved PCR amplification of genomic DNA from P. acnes of each group using a single random primer, or a combination of different random primers, which generated a banding pattern of PCR products. Multiple RAPD primers were tested to identify ones that would generate different banding patterns from P. acnes of Groups 1, 2 and 3 (see FIG. 5). Amplification of a particular PCR band from one P. acnes group but not from the other groups indicates a DNA sequence variation, which may be useful for design of group-specific primers.

### 2. Use of Step-Out PCR to Characterise Sequence Variations

Once RAPD-PCR bands that varied consistently between the different groups of P. acnes were identified, these bands were cut from the gels, purified, and the DNA sequence was determined to allow design of primers specific for those genomic regions. The DNA sequences were extended by a standard method of step-out PCR, using these specific primers in combination with universal primers based on endonuclease restriction sites (termed RS-PCR) as described by Sarkar et al (1993). Primers designed from these sequences were then used to obtain the corresponding region of DNA from the P. acnes groups which did not display the RAPD-PCR band under investigation. Comparison of the DNA sequences thus obtained from Groups 1, 2 and 3 in some cases identified sequence variations that could be used to design primers specific for particular P. acnes groups.

In other cases, several different sets of primers failed to amplify the corresponding DNA region from the P. acnes groups that did not display the RAPD-PCR band under investigation, suggesting that the genomic region was missing in the latter groups. In these cases the DNA sequence from the group possessing the genomic region under investigation was repeatedly extended by step-out PCR, and primers designed within each 500 nt of extended sequence were used to try and amplify this DNA from the other groups. Eventual amplification of DNA from the other groups with a primer set indicated that the end of the missing region had been reached. Primers were then designed in regions of DNA bracketing the missing regions. When the missing region was small, these primers generated PCR products of different sizes from the groups concerned, allowing each group to be distinguished by its PCR product size. Alternately when the missing region was large, a PCR product would only be produced from the groups with this region missing because the DNA region between the primers in the other groups was too large to be amplified by PCR. This allowed design of PCR primers specific for groups with the region of DNA missing. Conversely, design of primers within the missing region produced a PCR product only from groups that did not have this DNA missing.

A range of PCR primer pairs designed in this way are outlined in Table 1. P. acnes used to obtain DNA sequences were prostatic isolates 02-2703 (Group 1), 03-56 (Group 2) and 02-2753 (Group 3). Primers pairs designed from these sequences were then tested on all of our Group 1, 2 and 3 isolates to ensure that they amplified a product of the correct size from all members of each group. The extended DNA sequences were compared to those in Genbank using the NCBI “blastn” search, while translations of the DNA sequences into protein sequences using the NCBI “blastx” search were used to search for protein homology.

### Results

### 1. Primer Pairs to Detect or Distinguish Group 2/3

Several RAPD-PCR bands found in Group 1 but missing from both Groups 2 and 3 were analyzed.

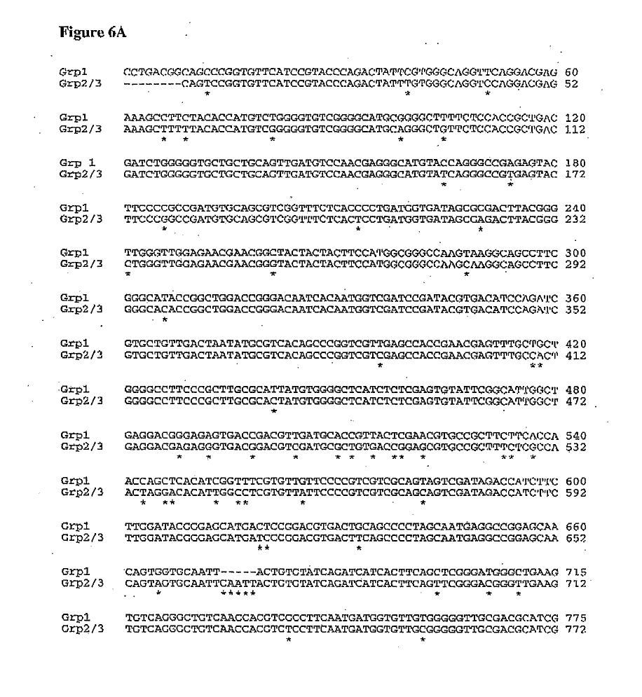

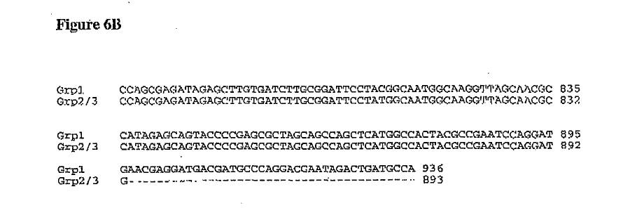

a) One band contained the downstream end of a beta-lactamase-like gene with marked sequence variations between Group 1 (SEQ ID NO:5) and Groups 2/3 (SEQ ID NO:6) (FIG. 6). These variations were used to design the primers G2/3F1 (SEQ ID NO:7), G2/3F2 (SEQ ID NO:9) and G2/3R (SEQ ID NO:8) that will selectively amplify DNA from P. acnes Groups 2 and/or 3. Optimization of this PCR assay is described in Example 4.

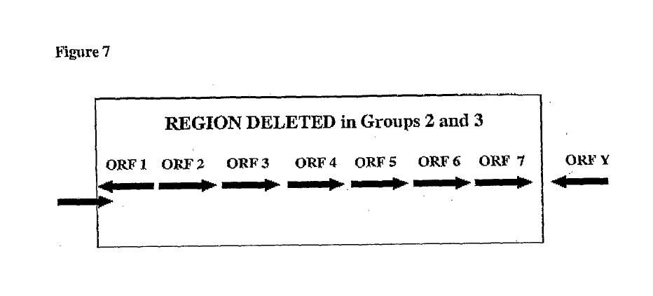

b) A second band proved to be part of a large 8693 nt DNA region present in Group 1 (SEQ ID NO:10) but missing from Groups 2 and 3. Southern blotting using a radioactive probe based on the Group 1 sequence indicated that this region of DNA is deleted from the genome of P. acnes Groups 2 and 3 (FIG. 5, Panel B). Analysis of this deleted DNA region (SEQ ID NO:10) with ORF-Finder software available at the NCBI website identified seven open reading frames (ORFs) that encoded proteins with significant homology to proteins in other bacterial species (Table 6). The chromosomal arrangement of these ORFs is shown in FIG. 8. These results indicate that the DNA region which is deleted in P. acnes Groups 2 and 3 (SEQ ID NO:10) contains a gene for N-acetyl-beta-hexosaminidase (an enzyme involved in breakdown of glycosaminoglycans), a five-gene operon for an ABC transporter system involved in uptake of peptides, and a gene for Chitinase (an enzyme involved in breakdown of chitin, a glucosamine in the exoskeleton of fungi and insects). The deleted region lies between a putative Endo-beta-mannanase gene at the upstream (5′) end and a putative Phosphopantetheine adenylyltransferase gene at the downstream (3′) end (FIG. 7).

ABC-type uptake transporter systems, which in bacteria are normally found in an operon of up to five sequential genes (Sutcliffe 1995), are involved in active import of nutrients (such as peptides, sugars and minerals) into the bacterial cell (Tam 1993). Bacteria with mutations/deletions in transporter systems tend to have different nutritional requirements to normal bacteria and/or are restricted in their ability to cope with normal environments (Jenkinson 1996, Borezec 2000). This finding may explain why Group 2 and 3 P. acnes predominate in the prostatic environment. Mutations/deletions in these transporter systems have also been linked to loss of hydrophobic cell surface adhesion proteins in other bacterial species (McNab 1998), possibly explaining why our Groups 2 and 3 P. acnes have a hydrophilic cell surface and do not bind together into aggregates like the hydrophobic Group 1 P. acnes in liquid culture medium.

TABLE 6
Significant Open Reading Frames identified in the DNA region
that is deleted in P. acnes Groups 2 and 3 (SEQ ID NO: 10).

| ORF | ¹Nucleotide No. | | | Closest Homology | | |
|---|---|---|---|---|---|---|
| No. | Start | End | Frame | Species | Gene | ²E Value |
| 1 | 1592 | 24 | −3 | Kineococcus | N-acelyl-beta-hexosaminidase | 1.0 × 10⁻⁸⁰ |
| | | | | radiotolerans | | |
| 2 | 1749 | 2873 | +1 | Vibrio vulnificus | Peptide ABC transporter, | 2.0 × 10⁻⁶¹ |
| | | | | | permease component | |
| 3 | 2876 | 3808 | +3 | Thermoanarerobacter | ABC-type peptide/nickel transporter, | 8.0 × 10⁻⁴⁸ |
| | | | | tengcongensis | permease component | |
| 4 | 3805 | 4896 | +2 | Vibrio vulnificus | Peptide ABC transporter, | 1.0 × 10⁻⁷⁸ |
| | | | | | ATP-binding component | |
| 5 | 4893 | 5924 | +1 | Pyrococcus furiosus | Oligopeptide ABC transporter, | 2.0 × 10⁶⁰ |
| | | | | | ATP-binding component | |
| 6 | 6015 | 7730 | +1 | Vibrio vulnificus | Peptide ABC transporter, | 3.0 × 10³² |
| | | | | | solute-binding component | |
| 7 | 7806 | 8642 | +1 | Coccidiodes immitis | Chitinase 3 | 5.0 × 10⁻⁵¹ |

¹Nucleotide numbering refers to SEQ ID NO: 10
²Significance of protein homology increases as the Error Value approaches zero.

DNA surrounding this 8693 nt deletion, was used to design the primers DELF (SEQ ID NO:12), DELR1 (SEQ ID NO:13) and DELR2 (SEQ ID NO:14) which bracket the missing region and amplify a 761 nt product from Groups 2 and 3 (SEQ ID NO:11). No product is obtained from Group 1 because the region between the primers is too large to amplify by PCR. Optimization of this PCR assay is described in Example 4.

c) A third band indicated a deletion of 605 nt in the Acetyl CoA Synthetase gene of Groups 2 and 3. DNA surrounding this 605 nt deletion was used to design the primers PR262 (SEQ ID NO:18) and PR263 (SEQ ID NO:19) which bracket the missing region and amplify a 901 nt product from Group 1 (SEQ ID NO:15), but a 295-296 nt product from Groups 2 (SEQ ID NO:16) and 3 (SEQ ID NO:17). PCR cycling conditions are 15 mins at 95° C., 35 cycles of 30 seconds at 94° C., 30 seconds at 58° C. and 1 min at 72° C., followed by 7 min extensional 72° C.

### 2. Primer Pairs to Detect Group 1.

DNA sequences within the 8693 nt DNA region present in Group 1 (SEQ ID NO:10) but missing from Groups 2 and 3 (described above) was used to design primer pairs specific for detection of Group 1 P. acnes.

a) Primers PR090 (SEQ ID NO:21) and PR108 (SEQ ID NO:22) amplify a443 nt product (SEQ ID NO:20) from Group 1 only. PCR cycling conditions are 15 mins at 95° C., 35 cycles of 30 seconds at 94° C., 30 seconds at 55° C. and 1 min at 72° C., followed by 7 min extension at 72° C.

b) Primers PR213 (SEQ ID NO:24) and PR216 (SEQ ID NO:25) amplify a 584 nt product (SEQ ID NO:23) from Group 1 only. PCR cycling conditions are 15 mins at 95° C., 35 cycles of 30 seconds at 94° C., 30 seconds at 58° C. and 1 min at 72° C., followed by 7 min extension at 72° C.

### 3. Primer Pairs to Detect or Distinguish Group 2.

Several RAPD-PCR bands either found only in Group 2, or missing only from Group 2 were analyzed.

a) One band showed an extra 102 nt within a putative UbiE gene of Group 2. DNA surrounding this extra 102 nt was used to design the primers PR217 (SEQ ID NO:29) and PR218 (SEQ ID NO:30). These primers bracket the extra region, amplifying a 545 nt product from Group 2 (SEQ ID NO:27) but smaller products of 443 nt from Group 1 (SEQ ID NO:26) and 440 nt from Group 3 (SEQ ID NO:28). PCR cycling conditions are 15 mins at 95° C., 35 cycles of 30 seconds at 94° C., 30 seconds at 55° C. and 1 min at 72° C., followed by 7 min extension at 72° C.

b) A second band showed a deletion of 208 nt within a putative Y4OU gene fragment in Group 1. DNA surrounding this missing 208 nt region was used to design the primers PR219 (SEQ ID NO:33) and PR220 (SEQ ID NO:34), which bracket the missing region and amplify a 716 nt product from Group 2 (SEQ ID NO:32) but a smaller 508 nt product from Group 1 (SEQ ID NO:31). No product is obtained from Group 3, indicating that Group 3 P. acnes either do not have this DNA region, or the sequence is too different to allow primer binding to occur. PCR cycling conditions are 15 mins at 95° C., 35 cycles of 30 seconds at 94° C., 30 seconds at 55° C. and 1 min at 72° C., followed by 7 min extension at 72° C.

c) A third band showed a deletion of 1957 nt in Group 2. DNA surrounding this missing 1957M region was used to design the primers PR221 (SEQ ID NO:37) and PR222 (SEQ ID NO:38), which bracket the missing region and amplify a 2279 nt product from Group 1 (SEQ ID NO:35) but a smaller 322 nt product from Group 2 (SEQ ID NO:36). No product is obtained from Group 3, indicating that Group 3 P. acnes either do not have this DNA region, or the sequence is too different to allow primer binding to occur. The missing DNA region has no significant nucleotide or protein homology with any sequences lodged in Genbank. PCR cycling conditions are 15 mins at 95° C., 35 cycles of 30 seconds at 94° C., 30 seconds at 55° C. and 1 min at 72° C., followed by 7 min extension at 72° C.

d) A fourth band showed a region where DNA sequences diverge in Groups 1 and 2. DNA surrounding this point of divergence was used to design primers PR256 (SEQ ID NO:40) and PR257 (SEQ ID NO:41) which bracket the point of divergence, with PR257 within DNA common to both groups but PR256 within adjacent. DNA present in Group 2 only. These primers produce a 725 nt product from Group 2 only (SEQ ID NO:39). No product is obtained from Group 3, indicating that Group 3 P. acnes either do not have this DNA region, or the sequence is too different to allow primer binding to occur. PCR cycling conditions are 15 mins at 95° C., 35 cycles of 30 seconds at 94° C., 30 seconds at 58° C. and 1 min at 72° C., followed by 7 min extension at 72° C.

e) A fifth band showed a region of DNA that appeared to be present only in Group 2. DNA sequences within this region were used to design primers PR253 (SEQ ID NO:43) and PR254 (SEQ ID NO:44), which amplify a 618 nt product (SEQ ID NO:42) flora Group 2 only. PCR cycling conditions are 15 mins at 95° C., 35 cycles of 30 seconds at 94° C., 30 seconds at 58° C. and 1 min at 72° C., followed by 7 min extension at 72° C.

### 4. Primer Pairs to Distinguish Group 3.

One RAPD-PCR band found only in Group 3 P. acnes was analyzed. This band showed a deletion of 3454 nt in Group 3, corresponding to partial loss of two putative RHS-family protein genes. DNA surrounding this missing 3454 nt region was used to design the primers PR245 (SEQ ID NO:47) and PR247 (SEQ ID NO:48), which bracket the missing region and may potentially amplify a 4027 nt product from Groups 1 and 2 (SEQ ID NO:45) but amplify a smaller 573 nt product from Group 3 (SEQ ID NO:46). PCR cycling conditions are 15 mins at 95° C., 35 cycles of 30 seconds at 94° C., 1 minute at 65° C. and 1 min at 72° C., followed by 7 min extension at 72° C.

### Example 4

### Development of PCR Assays to Selectively Detect DNA from P. acnes Groups 2 and 3 in Patient Samples

1. Preparation of Positive Control Samples from Patient Tissues

40 mg samples of frozen prostatic tissue were taken from a radical prostatectomy specimen that had previously proven negative for P. acnes DNA by PCR testing and were spiked with approximately 0, 10, 50, 100, 500, 1500, 5000 or 5 million colony-forming units (cfu) of Group 3 P. acnes before being homogenized in 128 μL of sterile PBS using the Mixer Mill (4 mins at 20 hz with a 5 mm sterile stainless steel ball bearing). After removal of the steel ball, the sample was processed as described in Example 1 for extraction of bacterial DNA.

2. Specificity and Sensitivity of P. acnes Group 2/3-Specific Beta-Lactamase-Based Primers

The Group 2/3-specific beta-lactamase-based PCR assay was performed as a semi-nested procedure to increase sensitivity. The first round of PCR involves primers G2/3F1 (SEQ ID NO:7) and G2/3R (SEQ ID NO:8) and thermal cycling conditions of 15 mins at 95° C., 25 cycles of 30 seconds at 94° C., 1 min at 66° C. and 1 min at 72° C., followed by 7 min extension at 72° C. HotStarTaq DNA Polymerase (Qiagen Pty Ltd) was used for these reactions. One μL of the first PCR reaction was used as template for the second round of PCR, which involves primers G2/3F2 (SEQ ID NO:9) and G2/3R (SEQ ID NO:8) and identical thermal cycling conditions except that the cycle number can be extended to 28-30 cycles. The final PCR product is 204 nt in size (within SEQ ID NO:6). Any PCR products obtained from clinical samples with this PCR assay can be confirmed as Group 2/3 P. acnes by sequencing of this product.

Specificity of the above primers was further assessed against a variety of other DNA sources using 30 cycles in the second round of PCR. These tests confirmed that they do not show any cross-reactivity with i) human DNA, ii) the endogenous bacterial DNA in HotStarTaq DNA polymerase, iii) a panel of Gram positive bacilli including Propionibacterium avidum and Propionibacterium granulosum, plus corynebacterium, actinomyces and bacillus species, or iv) DNA from any of the bacterial species isolated from prostatic tissues in this study (see Example 1) except for P. acnes.

Sensitivity tests involved PCR of the spiked positive control samples described above. This DNA extraction/PCR protocol could detect Group 3 P. acnes at a minimal concentration of 500 cfu/sample (12,500 cfu/gram) using a minimum of 28 cycles in the second round of PCR. No PCR product was obtained from the unspiked tissue or the 10, 50 or 100 cfu spiked samples even when higher cycle numbers were used for the second round of PCR.

3. Specificity and Sensitivity of P. acnes Group 2/3-Specific Deletion-Based Primers

The Group 2/3-specific deletion-based PCR assay was performed as a semi-nested procedure to increase sensitivity. The first round of PCR involves primers DELF (SEQ ID NO:12) and DELR1 (SEQ ID NO:13) and thermal cycling conditions of 15 mins at 95° C., 25 cycles of 30 seconds at 94° C., 1 min at 67° C. and 2 mins at 72° C., followed by 7 min extension at 72° C. HotStarTaq DNA Polymerase (Qiagen Pty Ltd) was used for these reactions. One μL of the first PCR reaction was used as template for the second round of PCR, which involves primers DELF (SEQ ID NO:12) and DELR2 (SEQ ID NO:14) and identical thermal cycling conditions except that the cycle number can be extended to 28 cycles. The final PCR product is 742 nt in size (within SEQ ID NO:11).

Specificity of the above primers was further assessed against a variety of other DNA sources using 27 cycles in the second round of PCR. These tests confirmed that they do not show any cross-reactivity with i) human DNA and ii) the endogenous bacterial DNA in HotStarTaq DNA polymerase. However, analysis of a panel of Gram positive bacilli including Propionibacterium avidum and Propionibacterium granulosum, plus corynebacterium, actinomyces and bacillus species and DNA from several bacterial species isolated from prostatic tissues in this study (see Example 1) showed that these primers can occasionally produce faint non-specific bands of varying sizes from pure genomic DNA of other bacteria. These faint PCR products are not 742 nt in size therefore can be distinguished from the PCR product obtained from P. acnes Group 2 or 3 by their size difference, or by sequencing of the DNA product.

Sensitivity tests involved PCR of the spiked positive control samples described above. This DNA extraction/PCR protocol could detect Group 3 P. acnes at a minimal concentration of 500 cfu/sample (12,500 cfu/gram) using 27 cycles in the second round of PCR. No PCR product was obtained from the unspiked tissue or the 10, 50 or 100 cfu spiked samples under these PCR conditions.

### Example 5

### Investigation into Whether P. acnes Isolates with Hydrophilic Cell Surfaces (Groups 2 and 3) Exist as a Minor Subtype on Human Facial Skin

Our previous work (Example 2) showed that 10 out of 12 (83%) of P. acnes isolates cultured from the prostatic tissue of prostate cancer patients had different, growth characteristics in liquid medium without agitation compared to common cutaneous P. acnes. These prostatic isolates (defined as P. acnes Groups 2 and 3) grew as a suspension even at high cell density, a characteristic that correlated with a hydrophilic cell surface. In order to determine whether P. acnes of Groups 2/3 might occur as a minor subgroup of the normal skin bacteria, samples obtained from the facial skin of a female volunteer were cultured as previously described (Example 2). Fifty colonies typical of P. acnes (small, slow-growing white/yellow colonies) were randomly selected for culture in brain-heart broth supplemented with 5% horse scrum. None of these 50 isolates showed the hydrophilic growth properties typical of Group 2/3 P. acnes from the prostate. All 50 isolates formed aggregates at high culture density and precipitated out of suspension leaving a clear supernatant, as we have previously observed for cutaneous P. acnes (all Group 1).

For 18 isolates DNA was extracted as described in Example 1. All 18 isolates were then confirmed as P. acnes by PCR and sequencing of the 16S rRNA gene (as described in Example 1) and also by analysis of RAPD-PCR banding patterns (as described in Example 3). All 18 isolates selected for genetic analysis showed the RAPD-PCR banding patterns typical of Group 1 P. acnes. Eight of the 18 isolates were randomly selected for PCR and DNA sequence analysis of the MMCoA gene (as described in Example 2), with all 8 shown to have SEQ ID NO:2 which is indicative of Groups 1 and/or 2. In addition, all 18 isolates were positive for PCR using primers PR090 (SEQ ID NO:21) and PR108 (SEQ ID NO:22) based within SEQ ID NO:10, indicating that none of these isolates have the 8693 nucleotide DNA deletion that is observed in P. acnes of Groups 2/3 (as discussed in Example 3). These isolates were all therefore typical of Group 1 P. acnes in having hydrophobic growth characteristics in liquid medium, Group 1 RAPD banding patterns, SEQ ID NO:2 and SEQ ID NO:10 (which is deleted in Group 2/3) plus negative PCR results with the beta-lactamase-based Group 2/3 PCR assay (as described in Example 4).

These results show that Group 1 P. acnes is commonly found on human facial skin. However we now understand that P. acnes Group 2 and 3 are less tolerant to oxygen levels in their growth environment and would have been selected against under the culture conditions used (which are outlined in Example 2). Had these been cultured under anaerobic conditions for the initial isolation from the skin prior to sub-culturing into the broth medium, some P. acnes Group 2/3 organisms may have been identified. Other studies show that serotype II P. acnes (which we believe are equivalent to our Group 3) constitute approximately 30% of P. acnes isolated from human facial skin. It is also likely that Group 2 P. acnes occur as a minor subtype on human skin because our analysis of the recently published complete genome sequence of a P. acnes skin isolate (Bruggemann 2004) shows that, the isolate used for genome sequencing (strain KFA171202) is in fact a Group 2 according to the genetic characteristics that we have determined for grouping of P. acnes. The serotype of strain KPA171202 is not mentioned by Bruggemann (2004).

### Example 6

### Comparison of P. acnes Groups 1, 2 and 3 with Known P. acnes Type Strains of Serotype I and Serotype II

The bacterial species Propionibacterium acnes is known to have two different serotypes, designated Serotype I and Serotype II, which can be distinguished using agglutination tests with human or animal antibodies (Ray 1970; Johnson 1972, Kishishita 1979). Very little published information is available on the differences between P. acnes Serotypes I and II, however the following facts are widely known (Ray 1970, Johnson 1972, Cummins 1975, Kishishita 1979, Sasaki 1980a):

i) Serotype I contains the sugar galactose in its cell wall, whereas Serotype II does not.
ii) Approximately 55-80% of Serotype I isolates ferment sorbitol, whereas no Serotype II strains are known to ferment this sugar.
iii) Serotype I is more common, constituting 70-80% of all P. acnes isolates cultured from human facial skin.

In addition, a recent study attempting to type clinical isolates of P. acnes using RAPD-PCR (Perry 2003) identified a particular 200 bp DNA band which appeared in the majority of isolates, including their Serotype I type strain, but was absent from their Serotype II type strain. They concluded that this RAPD banding pattern may prove useful in distinguishing between P. acnes of Serotypes I and II. By this classification, 16/23 (70%) of their clinical isolates cultured from prosthetic hip infections and microdissectomy tissue specimens from sciatica patients appeared to be Serotype I, which is consistent with the proportion of this serotype on human facial skin.

Although the serotype of our 18 P. acnes isolates (12 cultured from the prostatic tissue of prostate cancer patients and 6 isolated from human facial skin for comparison) is not known, sugar fermentation tests showed that 6/8 (75%) of Group 1 isolates and 4/5 (80%) of Group 2 isolates fermented sorbitol. In addition all Group 1 and 2 isolates analyzed by RAPD-PCR as described by Perry (2003) had the 200 bp DNA band that may be associated with Serotype I. In contrast, none of the 4 isolates from Group 3 fermented sorbitol or had the 200 bp RAPD band. These results indicated that P. acnes Groups 1 and 2 are probably Serotype I, whereas Group 3 may be Serotype II.

Two type, strains of P. acnes were purchased for comparison with our cultured isolates. These were ATCC 6919 (Serotype I) and ATCC 11828 (Serotype II). Genetic analysis revealed that the Serotype I strain 6919 was typical of our Group I isolates in the following characteristics:

i) A Group 1 banding pattern in all 3 RAPD-PCR assays that we have developed for typing of P. acnes (including the one previously developed by Perry (2003), with presence of the 200 bp band as discussed above),

### ii) Presence of MMCoA SEQ ID NO:2.

iii) Presence of the Group 1 beta-lactamase sequence (SEQ ID NO:5).
v) Presence of the 8693 nt region (SEQ ID NO:10)

Strain 6919 tacks the hydrophobic cell surface properties of our Group 1 isolates and docs not form aggregates then drop out of suspension to leave a clear supernatant when cultured in liquid medium without agitation. However, these cohesive/aggregating growth properties are frequently lost by bacterial isolates after prolonged laboratory culture, presumably because the selective forces that make cohesion and aggregation important properties for growth on human skin are no longer present in artificial growth conditions (Wyss 1989).

In contrast, the Serotype II type strain 11828 was found to be typical of our Group 3 isolates in the following characteristics:

i) A Group 3 banding pattern in all 3 RAPD-PCR assays that we have developed for typing of P. acnes (including the one previously developed by Perry (2003), with lack of the 200 bp band as discussed above).

### ii) Presence of MMCoA SEQ ID NO:1.

iii) Presence of the Group 2/3 beta-lactamase sequence (SEQ ID NO:6).
iv) Deletion of 8693 nt region (SEQ ID NO:10).
v) Hydrophilic growth properties, staying in suspension when cultured in liquid medium without agitation.

These results indicate that our Group 1 isolates are Serotype I, whereas our Group 3 isolates are Serotype II. The relationship of Group 2 P. acnes to the serotyping system is less certain. They are likely to be Serotype I because most Group 2 isolates ferment sorbitol and all have the 200 bp RAPD-PCR band associated with Serotype I. Genetically, Group 2 isolates also have MMCoA SEQ ID NO:2 as seen in Group 1/Serotype I strains.

However Group 2 isolates also share several characteristics with Group 3, including the Group 2/3 beta-lactamase sequence (SEQ ID NO:6), deletion of the 8693 nt region (SEQ ID NO:10) and the 605 nt deletion within the Acetyl CoA synthetase gene (SEQ ID NO:16) which is very similar to SEQ ID NO:17 of Group 3. By RAPD banding patterns (which give a broader indication of genetic similarity), Group 2 P. acnes are identical to Group 1/Serotype I in 2 of the 3 RAPD assays that we use for typing of P. acnes, whereas in the third assay they have a banding pattern partly the same as Group 1 and partly the same as Group 3. Group 2 therefore appears to be a genetic intermediate between Groups 1 and 3.

In this respect it is interesting to note that many P. acnes strains react with antiserum against both Serotype I and Serotype II P. acnes (Ray 1970; Johnson 1972); such strains may represent our Group 2. More recently, the phylogenetic differences between P. acnes (serotype I and (serotype n were discussed (McDowell 2005). They concluded that serotypes I and II are distinct phylogenetic groups that have been evolving separately for a long period of time. They also described a group of atypical P. acnes that would stain only weakly with antibodies that usually distinguish type I and type II, or would stain with both antibodies. Other genetic criteria suggested that these atypical strains may be a subtype within serotype I. We suggest that these atypical P. acnes are equivalent to the P. acnes Group 2 described herein. Our analysis of the genome sequences from P. acnes strain KPA171202 (Bruggemann 2004), which is a Group 2 according to the genetic characteristics that we have determined for grouping of P. acnes, shows that strain KPA171202 also falls into the “atypical” group believed by McDowell (2005) to be a subtype of serotype I (discussed above).

In terms of prostate disease, our results from Examples 1 and 2 show preferential involvement of P. acnes Groups 2 and 3 by culture analysis of prostatic tissue from prostate cancer patients. Although Serotype II is a minor type on human facial skin, our results suggest preferential involvement of Serotype II P. acnes (Group 3), or P. acnes with at least some characteristics of Serotype II (i.e. Group 2), in prostate disease. One similar finding has been reported for Serotype II P. acnes in a study of peridontal disease, where 55/80 (64%) of P. acnes isolated from root surface caries lesions and peridontal pockets were found to be Serotype II (Sasaki 1980b). This study reports that Serotype II P. acnes usually possess numerous long pili that are associated with higher haemagglutinating activity, indicating that that Serotype II may be better able to adhere to and therefore colonize (and cause disease of) mucous membranes. In contrast, Serotype I P. acnes were found to have a small number of short pili and weaker hemagglutinating activity (Sasaki 1980b).

### Example 7

### Method for Detecting DNA Regions Present in Group 3 but not in Groups 1 and 2

The purpose of this work is to use a representational difference analysis (RDA) technique to define areas of one genome that are absent in the genome of another. This is best used to demonstrate areas of deletion between cell types, or in our case different strains of bacteria, and is based on the method described by Hou P et al (1996) adapted from Lisitsyn N A et al (1995) and Lisitsyn and Wigler (1995).

Tester DNA is the DNA in which you wish to find the sequences that are not present or different from the other DNA, termed the Driver DNA. The method below can be used but is not limited to the following example.

Genomic DNA from a Group 3 bacterium (tester DNA) is isolated and cleaved with a restriction enzyme. The digestion reaction is then subjected to ligation with an adapter comprising two oligonucleotides, which when combined form the sticky ends of the restriction site, and will ligate to the cut ends of the DNA. For example see adapter I in Table 7 which comprises primer I and primer II. Enzymes used can include any enzyme that gives a 3′ or 5′ overhang and gives restriction fragments of a reasonable size for amplification. The method can also use a variety of different enzymes used separately to cover areas that may not be suitable for amplification by any specific enzyme. Tester DNA when ligated with the appropriate restriction enzyme specific adapters, is then mixed with Driver DNA (Group 1 isolate DNA or group 2 isolate DNA), that has been sonicated to give an average fragment size of between 500 and 1500 bp where driver DNA is in excess (>50:1). The mixture is denatured and reannealed after the addition of NaCl at 68° C. for 20 hr then cleaned up using Phenol Chloroform or other relevant clean up procedures. A portion of the mixture is incubated with Taq DNA polymerase in PCR mixture without the primer to till in overhangs after which primer I is added and amplification performed over about 20 cycles. This will selectively amplify fragments of DNA that appear only in the Tester DNA. This is referred to as the representational stage.

The resulting PCR products are redigested with the same restriction enzyme and ligated to a second but different adapter that creates the same restriction site but the remaining sequence is different from that in primers I and EC and can be used as an amplification primer Table 7. The procedure above is repeated and constitutes the second round of hybridization and is repeated again using a third different adapter for a third round and so on until distinct bands can be distinguished. These products can be cloned and or sequenced directly. This represents the amplification phase of the technique.

TABLE 7

Primers used as adapters and amplification primers for
representational differential display.

| | | | | |
|---|---|---|---|---|
| Adapter | Primer | Name | Sequence 5′-3′ | Stage Used |

| | | | | | |
|---|---|---|---|---|---|
| BamHI | Adapter I | Bam primerI | BSK-I-24 | AGCACTCTCCAGCCTCTCACCGAG | Representation |
| | | Bam primerII | BamHI-II2 | GATCCTCGGTGA | |
| | Adapter II | Bam primerIII | BSK-II-24 | ACCGACGTCGACTATCCAAGAACG | Amplification |
| | | Bam primerIV | BamIII-II12 | GATCCGTTCTTG | odd |
| | Adapter III | Bam primerV | BSK-III-24 | AGGCAACTGTGCTATCCGAGGGAG | Amplification |
| | | Bam primerVI | BamHI-III12 | GATCCTCCCTCG | even |
| SalI | Adapter I | Sal primerI | BSK-I-24 | AGCACTCTCCAGCCTCTCACCGAG | Representation |
| | | Sal primerII | SalI-I12 | TCGACTCGGTGA | |
| | Adapter II | Sal primerIII | BSK-II-24 | ACCGACGTCGACTATCCAAGAACG | Amplification |
| | | Sal primerIV | SalI-II12 | TCGACGTTCTTG | odd |
| | Adapter III | Sal primerV | BSK-III-24 | AGGCAACTGTGCTATCCGAGGGAG | Amplification |
| | | Sal primerVI | SalI-III12 | TCGACTCCCTCG | even |
| KasI | Adapter I | Kas primerI | BSK-I-24 | AGCACTCTCCAGCCTCTCACCGAG | Representation |
| | | Kas primerII | KasI-I12 | GCGCCTCGGTGA | |
| | Adapter II | Kas primerIII | BSK-II-24 | ACCGACGTCGACTATCCAAGAACG | Amplification |
| | | Kas primerIV | KasI-II12 | GCGCCGTTCTTG | odd |
| | Adapter III | Kas primerV | BSK-III-24 | AGGCAACTGTGCTATCCGAGGGAG | Amplification |
| | | Kas primerVI | KasI-III12 | GCGCCTCCCTCG | even |

This process can be undertaken using a variety of different restriction enzymes to generate a variety of fragments of an amplifiable size for PCR. Specific adapters will need to be designed for each restriction enzyme used.

### Example 8

### Method for Isolating P. acnes from Urine and Development of a Urine Test

Previous analysis of patient samples suggest that P. acnes is present only in very low numbers (Example 1). We have therefore developed a method of detection, which involves the use of bacterial culture to selectively amplify, the numbers of P. acnes present in patient samples prior to analysis by PCR. This method could be applied to any patient samples available for analysis, including urine samples as discussed in the example below.

### Methods

### 1. Preparation of Semi-Selective Medium

Brain Heart Infusion Agar (Oxoid Australia Ltd) was prepared containing 0.5% Tween 80 and adjusted to a pH of 6.0 using hydrochloric acid prior to autoclaving. After autoclaving the solution was cooled and the following ingredients were added: sterile horse serum (at 5%), filter-sterilized ascorbic acid (at 0.01%), furazolidone (Sigma) at 6 μg/mL and filter-sterilized gentamicin (Pharmacia) at 4 μg/mL. Furazolidone stock solution (1.33 mg/mL) was prepared by dissolving 40 mg of furazolidone powder in 30 mL of acetone at 37° C. with agitation for several hours. Both antibiotics were prepared freshly immediately prior to use, and the agar plates were incubated at 37° C. for 24 hrs to ensure sterility then stored at 4° C. and used within 3 weeks of preparation.

The use of furazolidone to prepare culture media semi-selective for Propionibacteria was previously developed by Smith (1969) and refined by Marino (1982), who reported that acidification to pH 6.1 prevents degeneration of the furazolidone into toxic intermediates and that addition of ascorbic acid improves the growth of P. acnes on this medium. Inclusion of gentamicin as a selective agent and Tween 80 as a growth stimulant for P. acnes was previously discussed by Kishishita (1980). The antibiotic concentrations of 6 μg/mL furazolidone and 4 μg/mL gentamicin were chosen because these were the maximal concentrations that allowed reasonable growth of our P. acnes Group 1, 2 and 3 isolates after 7 days of anaerobic incubation. Some strains of P. acnes Groups 2 and 3 showed noticeable growth inhibition even at these concentrations.

This medium is only semi-selective for P. acnes and still allows growth of many streptococcal species (discussed below) therefore future work will involve trying to improve its selectivity. Methods to be trialled will include lowering of the medium pH and addition of other substances that P. acnes is resistant to, including the metronidazole and sulfonamide classes of antibiotics.

2. Culture of Bacteria from Urine Samples

Approximately 20 mL of initial-stream urine was collected from 12 male patients about to undergo prostate biopsy for cancer diagnosis in view of an elevated serum PSA. Two mL of each urine sample was centrifuged at maximum speed for 10 minutes, the supernatant decanted and the pellet resuspended in 100 μL of sterile water then evenly plated onto the semi-selective agar plates described above. The plates were incubated under anaerobic conditions in an atmosphere generation jar at 37° C. for three days then examined for bacterial growth. At this stage no colonies of P. acnes are visible yet so all colonies present were marked as “not P. acnes” by placing a dot on the back of the agar plate corresponding to their position. This was done because many colonies did not continue to grow with continued incubation and if assessment was only performed after 7 days, these bacteria could not always be distinguished by either colony morphology or colony size from P. acnes colonies. At least two representative colonies of each distinguishable type present were taken for subculture and identification, then the plates were returned to anaerobic incubation for a further 4 days.

On the 7th day of incubation the plates were examined again for new, unmarked colonies.

For colonies typical of P. acnes (white colonies 1-2 mm in diameter) up to 30 isolates were taken for subculture and identification, while for other colony types at least two representative colonies were subcultured. All isolates were subcultured in Brain Heart Infusion broth with 5% horse serum, incubated at 37° C. without agitation for up to 2 weeks.

3. Identification of Bacteria from Urine Samples

DNA was extracted from bacterial broth subcultures by centrifuging 1 mL of turbid culture for 2 min at maximum speed, decanting the supernatant, washing the pellet once in 1 mL of sterile PCR-grade water, and then resuspending the pellet in 200 μL of sterile PCR-grade water. This was boiled for 10 mins, centrifuged at maximum speed for 4 mins to pellet the debris, and the supernatant containing bacterial DNA was used directly for PCR. While this method worked consistently for P. acnes cultures, it frequently failed to produce DNA suitable for PCR from other bacterial species, in these cases the DNA was extracted using bead-beating and the QiaAmp DNA mini kit as described for bacterial isolates in Example 1.

Bacterial isolates were identified as P. acnes or P. avidum by PCR using the primers MMF (SEQ ID NO:3) and MMR (SEQ ID NO:4) as described in Example 2. P. acnes isolates were then identified as Group 1, 2 or 3 by PCR using the primers PR219 and PR220 (as described in Example 3) to detect and distinguish Groups 1 and 2, and the primers PR245 and PR247 (as described in Example 3) to detect and Group 3. P. avidum does not give a positive PCR result with the latter two primer pairs. Other bacterial species (including P. avidum) were identified by sequencing of the 16S rRNA gene and comparison with 16S sequences in Genbank as described for bacterial isolates in Example 1.

4. Extraction of Bacterial DNA from “Plate-Scrape” Samples

After 7 days of anaerobic incubation, a comprehensive sample of bacterial colonies present was collected by scraping the agar plate with a sterile loop. Particular effort was made to sample all small colonies morphologically typical of P. acnes, and the remnants of colonies already sampled for subculture. This bacterial “plate-scrape” sample was resuspended in 128 μL of sterile PBS and the DNA was then extracted using bead-beating and the QiaAmp DNA mini kit as described in Example 1 for cultured bacterial isolates. The DNA was then analyzed by PCR using the three P. acnes-specific primers pairs as described above for cultured isolates.

### Results

7. Culture of P. acnes from Urine Samples

As expected, use of furazolidone in the semi-selective medium prevented growth of Gram-negative bacteria and Staphylococcal species from the urine samples. Streptococcal species were the predominant micro-organisms isolated from most patients (Table 8), and in many cases these were present in high enough numbers to inhibit the isolation of slow-growing bacteria by using up most of the available space and nutrients. However, P. acnes was cultured from 5 of the 12 patients analyzed and the predominant P. acnes type isolated from the urinary tract was Group 2 (grown from 4 patients; the numerically predominant group of P. acnes in 3 of these cases). Group 3 P. acnes were isolated from 2 patients and were present in relatively high numbers in one of these cases. Group 1 P. acnes were isolated from 3 patients but were present only in low numbers in each case. These results are summarized in Table 8. In all cases, the Group 2 and 3 P. acnes isolated from the urinary tract showed the same hydrophilic growth properties in liquid medium as observed for Group 2/3 P. acnes previously isolated from prostate tissue (discussed in Example 2). Similarly, all Group 1 isolates from the urinary tract showed the same aggregative/hydrophobic growth properties previously observed for Group 1 isolates from prostate tissue and human facial skin.

TABLE 8
Culture results from urine samples (2
mL) from 12 prostate biopsy patients

| | | P. acnes | |
|---|---|---|---|
| | | cultured | |
| Patient | | (% of no. | |
| No (cfu) | Predominant Species (cfu) | cultured) | P. acnes Group |
| 05-205 | Streptococcus ¹m/a (156) | No | — |
| 05-206 | Propionibacterium acnes | Yes (18) | Group 3 (66.67%) |
| | (18) | | Group 2 (16.67%) |
| | | | Group 1 (16.67%) |
| 05-266 | Enterococcus faecalis (41) | No | — |
| | Streptococcus ¹m/a (33) | | |
| 05-271 | Streptococcus ¹m/a (106) | No | — |
| 05-329 | No bacterial growth | No | — |
| 05-333 | Streptococcus ¹m/a (100) | Yes (1) | Group 2 (100%) |
| | Corynebacterium sp. (30) | | |
| 05-334 | Propionibacterium avidum | Yes (4) | Group 2 (75%) |
| | (5) | | Group 1 (25%) |
| | Propionibacterium acnes | | |
| | (4) | | |
| 05-335 | Streptococcus pneumoniae | No | — |
| | (247) | | |
| | Streptococcus ¹m/a (38) | | |
| 05-336 | Streptococcus mitis (38) | Yes (5) | Group 2 (80%) |
| | Streptococcus ¹m/a (15) | | Group 3 (20%) |
| 05-337 | Streptococcus agalactiae | No | — |
| | (115) | | |
| | Streptococcus ¹m/a (52) | | |
| 05-338 | Streptococcus ¹m/a (28) | No | — |
| 05-351 | Dermabacter hominis (28) | Yes (2) | Group 1 (100%) |
| | Propionibacterium | | |
| | propionicus (20) | | |

¹m/a = milleri or anginosus; the 16S rRNA gene sequence for these Streptococcus species is identical within the region analyzed by PCR.

2. PCR analysis of bacterial DNA from “plate-scrape” samples

Analysis of DNA from the combined bacterial colonies scraped from the agar plates was only trialled for the last 8 prostate biopsy patients, and one of these (05-329) was omitted because no bacterial growth appeared at all. PCR results from the plate-scrapes very closely reflected the P. acnes culture results for the seven patients analyzed (Table 9). The plate-scrape method was found to be more accurate for detection of the predominant P. acnes group present, as it identified P. acnes in two patients that were negative by culture analysis (Table 9). In one case (05-337) a large number of colonies was evident on the plate (see Table 8) however sampling of 20 small colonies did not pick up P. acnes, therefore it must have been present as a small minority. In the second case (05-338) only two new colonies grew on the plates after Day 3 of incubation; both of these were taken for subculture but neither grew in the broth. The plate-scrape method included sampling the remnants of these two colonies and indicates that they were Group 2 P. acnes. The loss of small colonies on subculture was a commonly encountered problem, and is typical of microacrophilic bacteria such as P. acnes (in particular Group 2, which grows more slowly) and often require a larger innoculum for growth to occur unless kept under strictly anaerobic conditions. Incubation of the broth bottles under anaerobic conditions could help with this problem.

While the plate-scrape method was found to be more accurate for detecting the predominant P. acnes group present, it was less sensitive for detection of additional P. acnes groups present in lower numbers. For patient 05-336 the presence of Group 3 was not detected by the plate-scrape method (Table 9). This is probably because the P. acnes DNA is “diluted” by the presence of DNA from multiple other colonies, and groups present as a small minority were beyond the sensitivity limits of the PCR assay used. This may be less of a problem if the plate-scrape method is used alone, rather than being preceded by sampling of the colonies present for subculture, which removes most of the colony from the agar plate.

TABLE 9
Comparison of culture results and “plate-scrape” results
from urine samples (2 mL) from 7 prostate biopsy patients.

| | | “Plate- | | |
|---|---|---|---|---|
| | Culture | scrape” | Groups | Groups |
| Patient | for | for | identified by | identified by |
| No. | P. acnes | P. acnes | culture | “plate-scrape” |
| 05-333 | Positive | Positive | Group 2 | Group 2 |
| 05-334 | Positive | Positive | Group 2 | Group 2 |
| | | | (majority) | (majority) |
| | | | Group 1 | Group 1 |
| | | | (minority) | (minority) |
| 05-335 | Negative | Negative | — | — |
| 05-336 | Positive | Positive | Group 2 (major) | Group 2 |
| | | | Group 3 (minor) | |
| 05-337 | Negative | Positive | — | Group 1 |
| 05-338 | Negative | Positive | — | Group 2 |
| 05-351 | Positive | Positive | Group 1 | Group 1 |

In conclusion, patient samples can be successfully assessed for presence of P. acnes using culture of the patient sample on a medium which selectively amplifies the numbers of P. acnes present in the sample, followed by identification using either subculture of individual colonies and/or the plate-scrape method. Our analysis of urine samples from patients about to undergo prostate biopsy shows that Group 2 is the predominant P. acnes type present in the urine of most patients. Further work is needed to determine whether the hydrophilic variants of P. acnes (Groups 2 and 3) are present as natural urethral flora in most males, or are predominantly present in urine from the group of men who develop prostate disease, including prostate cancer.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

Anderson P, Smith D H. (1977) Immunogenicity in weanling rabbits of a polyribophosphate complex from Haemophilus influenzae type b. J Infect Dis. 136:S63-70.
Berger R, Krieger J, Rothman I, et al. (1997) Bacteria in the prostate tissue of men with idiopathic prostatic inflammation. The Journal of Urology 157:863-865.
Blumenfeld W, Tucci S, Narayan P. (1992) Incidental lymphocytic prostatitis: selective involvement with non-malignant glands. The American Journal of Surgical Pathology 16:975-981.
Borezee E, Pellegrini E, Berche P (2000) OppA of Listeria monocytogenes, an oligopeptide-binding protein required for bacterial growth at low temperature and involved in intracellular survival. Infection and Immunity 68:7069-7077.
Brook I, Frazier E (1991) Infections caused by Propionibacterium species. Reviews of Infectious Diseases 13:819-822.
Brown M H, Wilson R N. (1959) Chronic bronchitis in industry; an account of a trial of H. influenzae vaccine. Br Med J. 34:263-7
Bruggemann H, Henne A, Hoster F, Liescgang H, Wiezer a, Sttrittmatter A, Hujer S, Durre P, Gottschalk G. (2004) The complete genome sequence of Propionibacterium acnes, a commensal of human skin. Science 305:671-673.
Cooper P, Millar M, Godwin P. (1988) Anaerobes and carcinoma of the prostate. British Medical Journal 296:466-467.
Cummins C (1975) Identification of Propionibacterium acnes and related organisms by precipitin tests with trichloroacetic acid extracts. Journal of Clinical Microbiology 2:104-110.
De Marzo A, Marchi V, Epstein J, et al (1999) Proliferative inflammatory atrophy of the prostate: implications for prostatic carcinogenesis. American Journal of Pathology 155(6): 1985-1992.
DeMarzo A, Nelson W, Isaacs W, et al (2003) Pathological and molecular aspects of prostate cancer. The Lancet 361:955-64.
Dennis L, Dawson, D. (2001) Meta-Analysis of measures of sexual activity and prostate cancer. Epidemiology 13(1):72-79.
Dudgeon L, Wallace C. (1904) A preliminary note on the bacteriological findings in seven cases of enlarged prostate. The British Medical Journal 2:1744-1745.
Esteban J, Garcia-Calvo G, Jimcnez-Castillo P, et al. (1996) Failure of gram stain to detect Propionibacterium acnes in specimens from clinically significant infections. Journal of Clinical Microbiology 34:2051.
Faraji E, Frank B. (2002) Multifocal atrophic gastritis and gastric carcinoma, Gastroenterol Clin North Am 32(2):499-516.
Franks L M. (1954) Atrophy and hyperplasia in the prostate proper. Journal of Pathology and Bacteriology 68:617-621.
Gerstenbluth R, Seftel A, MacLennan G, et al. (2002) Distribution of chronic prostatitis in radical prostatectomy specimens with up-regulation of Bcl-2 in areas of inflammation. The Journal of Urology 167:2267-2270.
Gorelick J, Senterfit E, Vaughan E. (1988) Quantitative bacterial tissue cultures from 209 prostatectomy specimens: findings and implications. The Journal of Urology 139:57-60.
Hall G, Pratt-Rippin K, Meisler D, et al. (1994) Growth curve for Propionibacterium acnes. Current Eye Research 13:465-466.
Hou P, Wang Z H, Wang XQm Wu M (1996) A simplified subtractive procedure for isolating deleted sequences. Nucleic Acids Research 24:2196-2197
Irani J, Levillain P, Goujon J-M, et al. (1997) Inflammation in benign prostatic hyperplasia: correlation with prostate specific antigen value. The Journal of Urology 157:1301-1303.
Jenkinson H, Baker R, Tannock G (1996) A binding-lipoprotein-dependent oligopeptide transport system in Streptococcus gordonii essential for uptake of hexa- and heptapeptides. Journal of Bacteriology 178: 68-77.
Johnson J, Cummins C (1972) Cell wall composition and deoxyribonucleic acid similarities among the anaerobic coryneforms, classical propionibacteria, and strains of Arachnia propionica. Journal of Bacteriology 109:1047-1066.
Jonsson P, Wadstrom T (1984) Cell surface hydrophobicity of Staphylococcus aureus measured by the salt aggregation test (SAT). Current Microbiology 10:203-210.
Kishishita M, Ushijima T, Ozaki Y, et al. (1979) Biotyping of Propionibacterium acnes isolated from normal human facial skin. Applied and Environmental Microbiology 38:585-589.
Kishishita M, Ushijima T, Ozaki Y, Ito Y. (1980) New medium for isolating Propionibacterium and its application to assay of normal human facial skin. Applied and Environmental Microbiology 40:51100-1105.
Kohler G, Milstein C. (1975) Continuous cultures of fused cells secreting antibody of predefined specificity. Nature 256:495-497.
Kohnen P, Drach G (1979) Patterns of inflammation in prostatic hyperplasia: a histologic and bacteriologic study. The Journal of Urology 121:755-760.
Lee J, Muller C, Rothman I, et al. (2003) Prostate biopsy culture findings of men with chronic pelvic pain syndrome do not differ from those of healthy controls. The Journal of Urology 169:584-588.
Lisitsyn N A, Lisitsina N M, Dalbagni G, Barker P, Sanchez C A, Gnarra J, Linmchan W M, Reid B J, Wigler M H (1995) Comparative genomic analysis of tumours; detection of DNA losses and amplification. PNAS 92:151-155
Lisitsyn and Wigler (1995) Representational difference analysis in detection of genetic lesions in cancer. Methods in Enzymology 254:291-304.
Lockhart D J, Dong H, Byrne M C, Follettle M T, Gallo M V, Chee M S, Mittmann M, Wang C, Kobayashi M, Horton H, Brown E L. (1996) Expression monitoring by hybridization to high-density oligonucleotide arrays. Nat. Biotechnol. 14:1675-1680.
Lucia M S, Torkko K C (2004) Inflammation as a target for prostate cancer chemoprevention: Pathological and laboratory rationale. The Journal of Urology 171:S30-35.
Makela P H, Peltola H, Kayhty H, Jousimies H, Peitay O, Ruoslahti E, Sivonen A, Renkonen O V. (1977) Polysaccharide vaccines of group A Neisseria meningitidis and Haemophilus influenzae type b: a field trial in Finland. J Infect Dis. 136 Suppl; S43-S50.
Marino C, Stoughtonb R B. (1982) Clinical use of a selective culture medium for wild and antibiotic-resistant Propionibacterium acnes. Am Acad Dermatol 6:902-908.
McCafferty J, Griffiths A D, Winter G, Chiswell D J. (1990) Phage antibodies: filamentous phage displaying antibody variable domains. Nature 348:552-554.
McClinton S, Miller I, Eremin O (1990) An immunohistochemical characteristic of the inflammatory cell infiltrate in benign and malignant prostatic disease. British Journal of Cancer 61:400-403.
McDowell A, Valanne S, Ramage G, Tunney M M, Glenn J V, McLorinan G C, Bhatia A, Maisonneuve J-F, Lodes M, Pcrsin D H and Patrick S. (2005) Propionibacterium acnes type I and II represent phylogenetically distinct groups. J Clin Micro 43:326-334
McGinley K, Webster G, Leyden J. (1978) Regional variations of cutaneous propionibacteria. Applied and Environmental Microbiology 35:62-66.
McNab R, Jenkinson H (1998) Altered adherence properties of a Streptococcus gordonii hppA (oligopeptide permease) mutant result from transcriptional effects on cshA adhesin gene expression. Microbiology 144:127-136.
McNcal J E, (1969) Origin and development of carcinoma in the prostate. Cancer 23:24-34.
Nam J M, Stoeva S I, Mirkin C A. (2004). Bio-bar-code-based DNA detection with PCR-like sensitivity. J Am Chem Soc. 126:5932-5933.
Nam J M, Thaxton C S, Mirkin C A. (2003) Nanoparticle-based bio-bar codes for the ultrasensitive detection of proteins. Science 26:1884-1886.
Needleman S B, Wunsch C D (1970) A general method applicable to the search for similarities in the amino acid sequence of two proteins. J Mol Biol 48:443-453.
Parke I C Jr, Schneerson R, Robbins J B, Schlesselman J J. (1977) Interim report of a controlled field trial of immunization with capsular polysaccharides of Haemophilus influenzae type b and group C Neisseria meningitidis in Mecklenburg county, North Carolina (March 1974-March 1976). J Infect Dls. 136:S51-S56.
Perry A, Worthington T, Hilton A, Lambert P, Stirling A, Elliot T (2003) Analysis of clinical isolates of Propionibacterium acnes by optimised RAPD. FEMS Microbiology Letters 228:51-55.
Platz E. (1998) Prostatitis and prostate cancer. New Dev Prostate Cancer Treatment 3: 71-73.
Platz E A, De Marzo A M (2004) Epidemiology of inflammation and prostate cancer. The Journal of Urology 171:S36-40.
Ray L, Kellum R (1970) Corynebacterium acnes from human skin: identification by morphologic, cultural, biochemical, serological, and chromatographic methods. Archives of Dermatology 101:36-40.
Relman D, Schmidt T, MacDermott R, et al. (1992) Identification of the uncultured bacillus of Whipple's disease. The New England Journal of Medicine 327:294-301.
Rosen R. (1918) The bacterial content of the prostate and its relation to prostatic adenoma. Journal of Infectious Diseases 24:63-75.
Rossi F, Torriani S and Dellaglio F (1998) Identification and clustering of dairy propionibacteria by RAPD-PCR and CGE-REA methods. The Journal of Applied Microbiology 85:956-964.
Sabel M, Felsberg J, Messing-Junger M, et al (1999) Glioblastoma multiforme at the site of metal splinter injury: a coincidence? Journal of Neurosurgery 91:1041-1044.
Sarkar G, Turner R T and Bolander M E (1993) Restriction-site PCR: a direct method of unknown sequence retrieval adjacent to a known locus by using universal primers. PCR Methods and Applications 2:318-322.
Sasaki N, Takazoc I. (1980a) Comparison of the biological characteristics in two serotypes of Propionibacterium acnes. Journal of Dental Research 59:1073.
Sasaki N, Nagai N, Takazoe I (1980b) Haemagglutinating activity of Propionibacterium acnes isolated from the human oral cavity. Bulletin of the Tokyo Dental College 21:49-61.
Silverman R. (2003) Implications for RNase L in prostate cancer biology. Biochemistry 42:1805-1812
Smith R (1969) A medium for the study of the ecology of human cutaneous diphtheroids. Journal of General Microbiology 57:411-417.
Smith C, Gardner W. (1987) inflammation-proliferation: possible relationships in the prostate. Prog Clin Biol Res 239:317-325.
Stirling A, Worthington T, Rafiq M, et al. (2001) Association between sciatica and Propionibacterium acnes. The Lancet 357:2024-2025.
Strickler H, Goeden J. (2001) Sexual behaviour and evidence for an infectious cause of prostate cancer. Epidemiological Reviews 23(1):144-151.
Sutcliffe I, Russell R (1995) Lipoproteins of Gram-positive bacteria. Journal of Bacteriology 177:1123-1128
Tarn R, Saicr M (1993) Structural, functional, and evolutionary relationships among extracellular solute-binding receptors of bacteria. Microbiological Reviews 57:320-346.
Tanner M, Shoskes D, Shahed A, et al. (1999) Prevalence of Corynebacterial 16S rRNA sequences in patients with bacterial and “non-bacterial” prostatitis. Journal of Clinical Microbiology 37(6): 1863-1870.
Thornton C, Kumar G, Haase C, et al. (1993) Primary structure of the monomer of the 12S subunit of Transcarboxylase as deduced from DNA and characterization of the product expressed in Escherichia coli. Journal of Bacteriology 175:5301-5308.
Ting T, Chern K, Meisler D, et al. (1999) Evaluation of Propionibacterium acnes isolates using contour-clamped homogeneous electric field gel electrophoresis. Anaerobe 5:579-582.
Torriani S, Zapparoli G, Dellaglio F (1999) Use of PCR-based methods for rapid differentiation of Lactobacillus delbrueckii subsp. bulgaricus and L. delbrucckii subsp. lactis. Appl Environ Microbiol. 65:4351-6.
Uemura N, Okamoto S, Yamamoto S, et al, (2001) Helicobacter pylori infection and the development of gastric cancer. New England Journal of Medicine 345(11):784-789.
Vancanncyt M, Lombardi A, Andrighetto C, Knijff E, Torriani S, Bjorkroth K J, Franz C M, Foulquie Moreno M R, Revets H, De Vuyst. L, Swings J, Kersters K, Dellaglio F, Holzapfel W H (2002) Intraspecies genomic groups in Enterococcus faecium and then correlation with origin and pathogenicity. Appl Environ Microbiol. 68:1381-91.
Van Oss C, (1978) Phagocytosis as a surface phenomenon. Annual Reviews in Microbiology 32:19-39.
Webster G. (1995) Inflammation in acne vulgaris. Journal of the American Academy of Dermatology 33:247-253.
Willen M, Hoist E, Myhre E. (1996) The bacterial flora of the genitourinary tract in healthy fertile men. Scandinavian Journal of Urology and Nephrology 30:387-393.
Wyss C (1989) Selected low-cohesion variants of Actinobacillus actinomycelemcomitans and Haemophilus aphrophilus lack distinct antigens recognized by human antibodies. Archives of Microbiology 151:133-136.
Xu J, Zheng L, Komiya A, et al, (2002) Germline mutations and sequence variants of the macrophage scavenger receptor 1 gene are associated with prostate cancer risk. Nature Genetics 32:321-325.
Yamada T, Eishi Y, Eceda S, et al. (2002) In situ localization of Propionibacterium acnes DNA in lymph nodes from sarcoidosis patients by signal amplification with catalysed reporter deposition. Journal of Pathology 191:541-547.

1. A method of diagnosing the presence of, or the predisposition to develop, prostate disease in a subject, the method comprising analysing a test sample from the subject for the presence of P. acnes infection of the prostate gland.

2. A method according to claim 1 wherein analysis of the test sample from the subject is by a method involving polymerase chain reaction (PCR) or culturing the test sample or a combination of both PCR and culturing the lest sample.

3. A method according to any one of claims 1 or 2 wherein the test sample is selected from the group comprising whole blood, serum, plasma, urine, semen, prostatic secretions and prostate tissue.

4. A method according to any one of claims 1 to 3 wherein the method distinguishes between P. acnes Group 1, Group 2 and Group 3 as herein defined, or combinations thereof.

5. An isolated polynucleotide, wherein the polynucleotide has a sequence selected from the group consisting of:
SEQ ID NO:1;
a sequence at least 99% identical to SEQ ID NO:1;
a fragment of at least about 10, at least about 15, at least about 20, at least about 25, at least about 30, at least about 50, at least about 100, at least about 150, at least about 200or more contiguous nucleotides of SEQ ID NO:1, wherein the sequence comprises a sequence that is not present in P. acnes Group 1 or that is specific for P. acnes Group 3;
a sequence which hybridizes to SEQ ID NO:1 under conditions of high stringency;
a sequence complementary to SEQ ID NO:1 or a fragment of SEQ ID NO:1 described above. SEQ ID NO:1;
SEQ ID NO:2;
a sequence at least 99% identical to SEQ ID NO:2;
a fragment of at least about 10, at least about 15, at least about 20, at least about 25, at least about 30, at least about 50, at least about 100, at least about 150, at least about 200or more contiguous nucleotides of SEQ ID NO:2, wherein the sequence comprises a sequence that is not present in P. acnes Group 3 or that is specific for P. acnes Group 1 or 2;
a sequence which hybridizes to SEQ ID NO:2 under conditions of high stringency;
a sequence complementary to SEQ ID NO:2 or a fragment of SEQ ID NO:2 described above.
SEQ ID NO:6;
a sequence at least 95%, preferably at least 96%, more preferably at least 97%, even more preferably at least 98%, and more preferably at least 99% identical to SEQ ID NO:6;
a fragment of at least about 10, at least about 15, at least about 20, at least about 25, at least about 30, at least about 50, at least about 100, at least about 150, at least about 200or more contiguous nucleotides of SEQ ID NO:6, wherein the sequence comprises a sequence that is not present in P. acnes Group 1 or that is specific for P. acnes Group 2 and/or Group 3;
a sequence which hybridizes to SEQ ID NO:6 under conditions of high stringency;
a sequence complementary to SEQ ID NO:6 or a fragment of SEQ ID NO:6 described above;
SEQ ID NO:11;
a sequence at least 95%, preferably at least 96%, more preferably at least 97%, even more preferably at least 98%, and more preferably at least 99% identical to SEQ ID NO:11;
a fragment of at least about 10, at least about 15, at least about 20, at least about 25, at least about 30, at least about 50, at least about 100, at least about 150, at least about 200or more contiguous nucleotides of SEQ ID NO:11, wherein the fragment comprises a sequence that is not present in P. acnes Group 1 or that is specific for P. acnes Group 2 and/or Group 3;
a sequence which hybridizes to SEQ ID NO:11 under conditions of high stringency;
a sequence complementary to SEQ ID NO:11 or a fragment of SEQ ID NO; 11 described above;
SEQ ID NO:15;
a sequence at least 95%, preferably at least 96%, more preferably at least 97%, even more preferably at least 98%, and more preferably at least 99% identical to SEQ ID NO:15;
a fragment of at least 10, at least 15, at least 20, at least 25, at least 30, at least 50, at least 100, at least 150, at least 200 or more contiguous nucleotides of SEQ ID NO:15, wherein the sequence comprises a sequence that is not present in P. acnes Group 2 and/or Group 3or that is specific for P. acnes Group 1;
a sequence which hybridizes to SEQ ID NO:15 under conditions of high stringency;
a sequence complementary to SEQ ID NO:15 or a fragment of SEQ ID NO:15 described above;
SEQ ID NO:16;
a sequence at least 95%, preferably at least 96%, more preferably at least 97%, even more preferably at least 98%, and more preferably at least 99% identical to SEQ ID NO:16;
a fragment of at least 10, at least 15, at least 20, at least 25, at least 30, at least 50, at least 100, at least 150, at least 200 or more contiguous nucleotides of SEQ ID NO:16, wherein the sequence comprises a sequence that is not present in P. acnes Group 1 and/or Group 3of that is specific for P. acnes Group 2;
a sequence which, hybridizes to SEQ ID NO:16 under conditions of high stringency;
a sequence complementary to SEQ ID NO:16 or a fragment of SEQ ID NO:16 described above;
SEQ ID NO:17;
a sequence at least 95%, preferably at least 96%, more preferably at least 97%, even more preferably at least 98%, and more preferably at least 99% identical to SEQ ID NO; 17;
a fragment of at least 10, at least 15, at least 20, at least 25, at least 30, at least 50, at least 100, at least 150, at least 200 or more contiguous nucleotides of SEQ ID NO: 17, wherein the sequence comprises a sequence that is not present in P. acnes Group 1 and/or Group 2 or that is specific for P. acnes Group 3;
a sequence which hybridizes to SEQ ID NO:17 under conditions of high stringency;
a sequence complementary to SEQ ID NO:17 or a fragment of SEQ ID NO:17 described above;
SEQ ID NO:20;
a sequence at least 95%, preferably at least 96%, more preferably at least 97%, even more preferably at least 98%, and more preferably at least 99% identical to SEQ ID NO:20;
a fragment of at least 10, at least 15, at least 20, at least 25, at least 30, at least 50, at least 100, at least 150, at least 200 or more contiguous nucleotides of SEQ ID NO:20, wherein the sequence comprises a sequence that is not present in P. acnes Group 2 and/or Group 3or that is specific for P. acnes Group 1;
a sequence which hybridizes to SEQ ID NO:20 under conditions of high stringency;
a sequence complementary to SEQ ID NO:20 or a fragment of SEQ ID NO:20 described above;
SEQ ID NO:23;
a sequence at least 95%, preferably at least 96%, more preferably at least 97%, even more preferably at least 98%, and more preferably at least 99% identical to SEQ ID NO:23;
a fragment of at least 10, at least 15, at least 20, at least 25, at least 30, at least 50, at least 100, at least 150, at least 200 or more contiguous nucleotides of SEQ ID NO:23, wherein the sequence comprises a sequence that is not present in P. acnes Group 2 and/or Group 3or that is specific for P. acnes Group 1;
a sequence which hybridizes to SEQ ID NO:23 under conditions of high stringency;
a sequence complementary to SEQ ID NO:23 or a fragment of SEQ ID NO:23 described above;
SEQ ID NO:26;
a sequence at least 95%, preferably at least 96%, more preferably at least 97%, even more preferably at least 98%, and more preferably at least 99% identical to SEQ ID NO:26;
a fragment of at least 10, at least 15, at least 20, at least 25, at least 30, at least 50, at least 100, at least 150, at least 200 or more contiguous nucleotides of SEQ ID NO:26, wherein the sequence comprises a sequence that is not present in P. acnes Group 2 and/or Group 3or that is specific for P. acnes Group 1;
a sequence which hybridizes to SEQ ID NO:26 under conditions of high stringency;
a sequence complementary to SEQ ID NO:23 or a fragment of SEQ ID NO:23 described above;
SEQ ID NO:27;
a sequence at least 95%, preferably at least 96%, more preferably at least 97%, even more preferably at least 98%, and more preferably at least 99% identical to SEQ ID NO:27;
a fragment of at least 10, at least 15, at least 20, at least 25, at least 30, at least 50, at least 100, at least 150, at least 200 or more contiguous nucleotides of SEQ ID NO:27, wherein the sequence comprises a sequence that is not present in P. acnes Group 1 and/or Group 3or that is specific for P. acnes Group 2;
a sequence which hybridizes to SEQ ID NO:27 under conditions of high stringency;
a sequence complementary to SEQ ID NO:27 or a fragment of SEQ ID NO:27 described above;
SEQ ID NO:28;
a sequence at least 95%, preferably at least 96%, more preferably at least 97%, even more preferably at least 98%, and more preferably at least 99% identical to SEQ ID NO:28;
a fragment of at least 10, at least 15, at least 20, at least 25, at least 30, at least 50, at least 100, at least 150, at least 200 or more contiguous nucleotides of SEQ ID NO:28, wherein the sequence comprises a sequence that is not present in P. acnes Group 1 and/or Group 2 or that is specific for P. acnes Group 3;
a sequence which hybridizes to SEQ ID NO:28 under conditions of high stringency;
a sequence complementary to SEQ ID NO:28 or a fragment of SEQ ID NO:28 described above;
SEQ ID NO:31;
a sequence at least 95%, preferably at least 96%, more preferably at least 97%, even more preferably at least 98%, and more preferably at least 99% identical to SEQ ID NO:31;
a fragment of at least 10, at least 15, at least 20, at least 25, at least 30, at least 50, at least 100, at least 150, at least 200 or more contiguous nucleotides of SEQ ID NO:31, wherein the sequence comprises a sequence that is not present in P. acnes Group 2 and/or Group 3 or that is specific for P. acnes Group 1;
a sequence which hybridizes to SEQ ID NO:31 under conditions of high stringency;
a sequence complementary to SEQ ID NO:31 or a fragment of SEQ ID NO:31 described above;
SEQ ID NO:32;
a sequence at least 95%, preferably at least 96%, more preferably at least 97%, even more preferably at least 98%, and more preferably at least 99% identical to SEQ ID NO:32;
a fragment of at least 10, at least 15, at least 20, at least 25, at least 30, at least 50, at least 100, at least 150, at least 200 or more contiguous nucleotides of SEQ ID NO:32, wherein the sequence comprises a sequence that is not present in P. acnes Group 1 and/or Group 3or that is specific for P. acnes Group 2;
a sequence which hybridizes to SEQ ID NO:32 under conditions of high stringency;
a sequence complementary to SEQ ID NO:32 or a fragment of SEQ ID NO:32 described above;
SEQ ID NO:35;
a sequence at least 95%, preferably at least 96%, more preferably at least 97%, even more preferably at least 98%, and more preferably at least 99% identical to SEQ ID NO:35;
a fragment of at least 10, at least 15, at least 20, at least 25, at least 30, at least 50, at least 100, at least 150, at least 200 or more contiguous nucleotides of SEQ ID NO:35, wherein the sequence comprises a sequence that is not present in P. acnes Group 2 and/or Group 3 or that is specific for P. acnes Group 1;
a sequence which hybridizes to SEQ ID NO:35 under conditions of high stringency;
a sequence complementary to SEQ ID NO:35 or a fragment of SEQ ID NO:35 described above;
SEQ ID NO:36;
a sequence at least 95%, preferably at least 96%, more preferably at least 97%, even more preferably at least 98%, and more preferably at least 99% identical to SEQ ID NO:36;
a fragment of at least 10, at least 15, at least 20, at least 25, at least 30, at least 50, at least 100, at least 150, at least 200 or more contiguous nucleotides of SEQ ID NO:36, wherein the sequence comprises a sequence that is not present in P. acnes Group 1 and/or Group 3 or that is specific for P. acnes Group 2;
a sequence which hybridizes to SEQ ID NO:36 under conditions of high stringency;
a sequence complementary to SEQ ID NO:36 or a fragment of SEQ ID NO:36 described above;
SEQ ID NO:39;
a sequence at least 95%, preferably at least 96%, more preferably at least 97%, even more preferably at least 98%, and more preferably at least 99% identical to SEQ ID NO:39;
a fragment of at least 10, at least 15, at least 20, at least 25, at least 30, at least 50, at least 100, at least 150, at least 200 or more contiguous nucleotides of SEQ ID NO:39, wherein the sequence comprises a sequence that is not present in P. acnes Group 1 and/or Group 3 or that is specific for P. acnes Group 2;
a sequence which hybridizes to SEQ ID NO:39 under conditions of high stringency;
a sequence complementary to SEQ ID NO:39 or a fragment of SEQ ID NO:39 described above;
SEQ ID NO:42;
a sequence at least 95%, preferably at least 96%, more preferably at least 97%, even more preferably at least 98%, and more preferably at least 99% identical to SEQ ID NO:42;
a fragment of at least 10, at least 15, at least 20, at least 25, at least 30, at least 50, at least 100, at least 150, at least 200 or more contiguous nucleotides of SEQ ID NO:42, wherein the sequence comprises a sequence that is not present in P. acnes Group 1 and/or Group 3or that is specific for P. acnes Group 2;
a sequence which hybridizes to SEQ ID NO:42 under conditions of high stringency;
a sequence complementary id SEQ ID NO:42 or a fragment of SEQ ID NO:42 described above;
SEQ ID NO:45;
a sequence at least 95%, preferably at least 96%, more preferably at least 97%, even more preferably at least 98%, and more preferably at least 99% identical to SEQ ID NO:45;
a fragment of at least 10, at least 15, at least 20, at least 25, at least 30, at least 50, at least 100, at least 150, at least 200 or more contiguous nucleotides of SEQ ID NO:45, wherein the sequence comprises a sequence that is not present in P. acnes Group 3 that is specific for P. acnes Group 1 or Group 2;
a sequence which hybridizes to SEQ ID NO:45 under conditions of high stringency;
a sequence complementary to SEQ ID NO:45 or a fragment of SEQ ID NO:45 described above;
SEQ ID NO:46;
a sequence at least 95%, preferably at least 96%, more preferably at least 97%, even more preferably at least 98%, and more preferably at least 99% identical to SEQ ID NO:46;
a fragment of at least 10, at least 15, at least 20, at least 25, at least 30, at least 50, at least 100, at least 150, at least 200 or more contiguous nucleotides of SEQ ID NO:46, wherein the sequence comprises a sequence that is not present in P. acnes Group 1 or Group 2 that is specific for P. acnes Group 3:
a sequence which hybridizes to SEQ ID NO:46 under conditions of high stringency;
a sequence complementary to SEQ ID NO:46 or a fragment of SEQ ID NO:46 described above.

6. A primer wherein the primer binds specifically to a polynucleotide of claim 5.

7. A primer according to claim 6 wherein the primer is selected from the group consisting of SEQ ID NO. 3, SEQ ID NO. 4, SEQ ID NO. 7, SEQ ID NO. 8, SEQ ID NO. 9, SEQ ID NO. 12, SEQ ID NO. 13, SEQ ID NO. 14, SEQ ID NO. 18, SEQ ID NO. 19, SEQ ID NO. 21, SEQ ID NO. 22, SEQ ID NO. 24, SEQ ID NO. 25, SEQ ID NO. 29, SEQ ID NO. 30, SEQ ID NO. 33, SEQ ID NO. 34, SEQ ID NO. 37, SEQ ID NO. 38, SEQ ID NO. 40, SEQ ID NO. 41, SEQ ID NO. 43, SEQ ID NO. 44, SEQ ID NO. 47 and SEQ ID NO. 48.

8. A primer sequence that distinguishes between Group 1, Groups 2 and Group 3 P. acnes as herein defined.

9. A primer according to claim 8 wherein the primer sequence specifically binds to SEQ ID NO. 1, SEQ ID NO. 2, SEQ ID NO. 6, SEQ ID NO. 11, SEQ ID NO. 15, SEQ ID NO. 16, SEQ ID NO. 17, SEQ ID NO. 20, SEQ ID NO. 23, SEQ ID NO. 26, SEQ ID NO. 27, SEQ ID NO. 25, SEQ ID NO. 31, SEQ ID NO. 32, SEQ ID NO. 35, SEQ ID NO. 36, SEQ ID NO. 39, SEQ ID NO. 42, SEQ ID NO. 45 and SEQ ID NO. 46.

10. A probe specific for P. acnes, wherein the probe detects or localizes a P. acnes nucleic acid or antigen.

11. A probe according to claim 10 wherein the probe detects or diagnoses a prostate disease or condition.

12. A probe according to any one of claim 10 or 11 wherein the probe is specific for P. acnes Group 2 and/or Group 3 as herein defined.

13. A probe according to any one of claims 10 to 12 wherein the probe is a nucleic acid, antibody, domain antibody or fragment thereof.

14. A kit for diagnosing the presence of, or the predisposition to develop, prostate disease in a subject, the kit comprising at least one P. acnes specific probe as defined herein.

15. A method of screening for an agent that has inhibitory effect on P. acnes, wherein the method comprises incubating P. acnes in the presence of an agent and detecting inhibitory effect of the agent on P. acnes.

16. A method of preventing or healing a prostate disease in a subject, the method comprising administering to a subject in need thereof an effective amount of a P. acnes inhibitory composition.

17. An isolated subtype of P. acnes Group 2 or Group 3 as herein defined


SEQUENCE LISTING (52 sequences)

SEQ ID NO: 1 - DNA, 633 bp, Propionibacterium acnes
cgggtacggcaagatgttctacgcgaacgtcaagctgtcaggcgtcgtgccgcagatcgccattattgctggcccctgcgccggtggcgcctcctattccccggccctgaccgacttcatcatcatgacgaagaaggcccacatgttcattacgggccccggcgtcatcaaatcggttaccggtgaggaggtgactgctgacgacctgggtggtgcggatgcgcacatgtccacctcgggcaatatccacttcgtggccgaagatgacgacgccgcagtgctcatcgcgcagaagttgctgagcttcctgccgcaaaacaacactgaggacgcccagatctccaatcccaatgacgatgtctccccgcagcctgagctgcgcgacatcgttccgctggacggtaagaagggctacgacgtccgcgacgtcatttccaagatcgtcgactggggcgactacctagaggtcaaggccggttgggcgaccaacatcatcaccgccttcgcccgggtcaatggtcgtaccgtcggcatcgtggccaaccagccgaaggtgatgtcgggttgccttgacattaatgcttcggacaaggctgccgagttcattaccttctgcgactcgttcaatattcc

SEQ ID NO: 2 - DNA, 633 bp, Propionibacterium acnes
cgggtacggcaagatgttctacgcgaacgtcaagctgtcgggcgtcgtgccgcagatcgccatcattgctggcccctgcgccggcggcgcctcctattccccggccctgaccgacttcatcatcatgacgaagaaggcccacatgttcattacgggccccggagtcatcaagtcggttaccggtgaggaggtgactgctgacgacctgggtggtgcggatgcgcacatgtccacctcgggcaatatccacttcgtggccgaagatgacgacgccgcagtgctcatcgcgcagaagttgctgagcttcctgccgcaaaacaacactgaggacgcccagatctccaaccccaatgacgatgtctccccgcagcctgagctgcgcgacatcgttccgctggatggtaagaagggctacgacgtccgcgacgtcatctccaagatcgtcgactggggcgactacctagaggtcaaggccggttgggcgaccaacatcgtcaccgcctttgcccgggtcaatggtcgtaccgtcggcatcgtggccaaccagccgaaggtgatgtcgggttgccttgacatcaatgcttcggacaaggctgccgagttcattaccttctgcgactcgttcaatattcc

SEQ ID NO: 3 - DNA, 20 bp, Propionibacterium acnes
cgggtacggcaagatgttct

SEQ ID NO: 4 - DNA, 22 bp, Propionibacterium acnes
ggaatattgaacgagtcgcaga

SEQ ID NO: 5 - DNA, 936 bp, Propionibacterium acnes
cctgacggcagcccggtgttcatccgtacccagactattcgtgggcaggttcaggacgagaaagccttctacaccatgtctggggtgtcggggcatgcggggcttttctccaccgctgacgatctgggggtgctgctgcagttgatgtccaacgagggcatgtaccagggccgagagtacttccccgccgatgtgcagcgtcggtttctcacccctgatggtgatagcgcgacttacgggttgggttggagaacgaacggctactactacttccatggcgggccaagtaaggcagccttcgggcataccggctggaccgggacaatcacaatggtcgatccgatacgtgacatccagatcgtgctgttgactaatatgcgtcacagcccggtcgttgagccaccgaacgagtttgctgctggggccttcccgcttgcgcattatgtggggctcatctctcgagtgtattcggcattggctgaggacgggagagtgaccgacgttgatgcaccgttactcgaacgtgccgcttcttcaccaaccagctcacatcggtttcgtgttgttccccgtcgtcgcagtagtcgatagaccatcttcttggatacgggagcatgactccggacgtgactgcagcccctagcaatgaggccggagcaacagtggtgcaattactgtgtatcagatcatcacttcagctcgggatgggctgaagtgtcagggctgtcaaccacgtcgccttcaatgatggtgttgtgggggttgcgacgcatcgccagcgagatagagcttgtgatcttgcggattcctacggcaatggcaaggttagcaacgccatagagcagtaccccgagcgctagcagccagctcatggccactacgccgaatccaggatgaacgaggatgacgatgcccaggacgaatagactgatgcca

SEQ ID NO: 6 - DNA, 300 bp, Propionibacterium acnes
gccaccgaacgagtttgccactggggccttcccgcttgcgcactatgtggggctcatctctcgagtgtattcggcattggctgaggacgagagggtgacggacgtcgatgcgctgtgaccggagcgtgccgctttctcgccaactaggacacattggcctcgtgttattccccgtcgtcgcagcagtcgatagaccatcttcttggatacgggagcatgatcccggacgtgacttcagcccctagcaatgaggccggagcaacagtagtgcaattcaattactgtgtatcagatcatcac

SEQ ID NO: 7 - DNA, 20 bp, Propionibacterium acnes
gccaccgaacgagtttgcca

SEQ ID NO: 8 - DNA, 28 bp, Propionibacterium acnes
gtgatgatctgatacacagtaattgaat

SEQ ID NO: 9 - DNA, 20 bp, Propionibacterium acnes
acgtcgatgcgctgtgaccg

SEQ ID NO: 10 - DNA, 8693 bp, Propionibacterium acnes
  misc_feature 3593, 5175: n = A,T,C or G
gcgcgcttcgtcccacccctgggctcacagaccgagaagccctcgccagtgctcgagcccactgaccggcttggctgcaacctcctcataatcagcccctcccgggagcctggccatggatcgtgccaggcctctgggtcggctggttcgtaaccacgcagtggcaaaacgatccaggagagccggggacaccgtcagttctcccccggtcaggacgtcgaggctgtggcagacaaggtccagaccgatacgcacgtcttccacccacccgatcgggccgccaccgagggcagcctcagtctcgttcgcgcggtcgtggagtcgagcaatttctgcacgggtggcagcgatcccgtcagctcccaggtcatcgcctgggtccccttccagcaggcaccgagccggcagtgaatggttacgtcgtatgacaccgagcttgtcctccagcagacctgctccgacgagcaggtgcgcaccagggtgatcatcgacgtcgaacagcgcctgcgacaccgcgttgacaagggatttagccgagttcagcccttcggcatggtccggatccgccatcgcgacgccagtgacgacaagaggaacgagagagaagacgagtcccgcatgatgtccgctgtctccccaacccgtgagcaagacccccttgccacccagggagtgatcggtgatgacggcgtcgacgatattgccgacggcgttgctcagacgtccggtgaaagtgttccacgctccagttccgggagcattcacgaacggccgaccagcattggcaacgagacgggcggtggacgcgaatccggcatcagaacgaatctcgtaaccttccgtgccagcgaacttggcttccatcgcgggggactcataagtccacgggatgagcaggacctcatcgggaatgatgtcgagtgcttcaggatgggaattgacgatgtcagcccacatggcaacccgccagcccctgtcgagccatggggtgacgacgtcgcgcgcccatttcgcgtacacaacccccttgccctcatctcggcaccgatcagcactgacaccggtgcccagctcccacggctcgtccagacctacgttgagaaacttcgacgagacagtctccgtcagttcgcccaacaacctctgaacgaagtcaagattctccggcactggagccaaggtgcttgccctggcatgaacgccccaggggaggtcgacgccgtcgggattctcacaccgatggtgatgggactcggtttccaaccacggttccatgtgacccagacagttctggttggccccgagctcgatgcctcgtccgcgacacagaccatcgagccacctcatgtcctccgccgtcacggggctgcggtcggcccacacctcctcctcaccgcggtaggcaaatcccgcttcaccatagagctcaaggtgattgagaccgcaccgcaccatgacgcgcgtcaggaaatcaagagtggaacgcgtcggcacacgatcacagctgacatcgagcatgaagccacggaaactgtcgtcgtatcgtgcagcattcacaggaaaccaactagttcccgcagacatcgtcgtcatgcggacaacctccacgtcattgtcgagaaattgatacgtcaatcgtgttgtacccgttcgcagtccggtgtatcttcctaacggcaggtaaaccgtttcagttgacgacgaagtcgaccggccatgacctgattcacgaaggggtgattacactatatgaccagtccaggtcaccctgagcctaccgctcaggagctcccgaccacggctgaccccgctgtggacccgtcgcatgcgcaaccccagacctcggggcgctcgcggcgtccacatcgtcgtggcggcggagcgttccttctcaagcggctgggcacgtacctgatagtcgccgtcgtgtcagtgttgctgaattttatcctgccgcgcttcatgccgggcgatcccgccacccagctcatcaaggacatcgtggccaagaccggcgccccgccatcgggctaccagatgcgcctcatcaaggcgcgctacggtgatcccgatcgctcggttcttctccagttctgggactacctcgttcagttggcccacggagacctcggaaagtccatcgagtactaccccgtcaacgtcacgaccctcatcaaccaggctctaccatggaccttgtacctggccatcgcctcgacgatcctcggttggatcgttggaacctggttgggtgtccgactggggtggaagcccggacgcaagctcgactcgatcgtcacccccttatcgatgttcttcagctcgatcccagccttctggctcggcctgctcatcgtatggtacttgcgctacaagaacggctggttccccaaccagggcgcctacgacaacgagttgacgatgagcctgaccaacggccacttcatcgcatcggtgctcatgcactcggtacttccgttcttcacccttgtcatcgtcgggttcgcaggctggctgtttagcatgcgcaacatgatgatcaccaccgtcaacgaggattatgtccagctcgcccgcgccaagggtctgtcgtcctcgcgcattcgcaatcgctacgccgcgcgtaacgccatccttcccaacttcaccggactcgctcagagcatcggtggctccttgacggccgtcattctggccgaggcggtgttcatctatcccggtatcggctccctcctctcgggcgctcagggccatcgtgactacccggtcatgcagggaatcatgctgatgatcatcttcatgagcctgctgttcaacttcatcgcggactcggtctacgtcatcctcgatccacggactcgagaggaggcctgataatggtcaaaaggttcctcggagtcgggaaggtgcaggtcggtctcgcgatcatgctcttcttcatcctggtcgccatcctcgggcagcccttctgcacccatgtgctgcataccagcccttaccaggtcgactacatgactctgggcggtacagcccccggaggcaaacactggctgggcaccaccagcgccggacaggacgtcctggcatggatgctgtacggcacccgaaactcggtggtcgtcggtctagcctccgccgtcatcggaacggtcctgaccgtcgtcatcgggacctgggccggattctccggcggctggattgaccgcttcctcaatggattcatcctggtcttcgccaacatcccgaccttcgccatcctgttcatgatcgccggtgtcatgcagaacgcgggctggctgctggtgtccctcgtcattggctgcttcgaatggtccggcggtgcccgtcagatccgcgcccaggcaatgtccctgcgaggacgcgatttcaccacggccctgcggaccatcggtgaatcgcagtcccacatcgtgctctcagaggtcatgccgcacctgctcggcctcatctccccgatgttcctgcgactcatcgccgccggcgtcaacatgcaggcgtccctagccttcctcggtatcggtgacccgtccatgccgtcctggggcctcatgatcaactgggccatgacgcanaacgcgctgttccgcggtctgtggtggtggttcattcctccgggtctcgcactcgcccttatcggtttcgcaaccaccatgatcaacttcggtctcgacgaggttaccaacccgacgctgtcgaccaagcgtatgaagctcatgcgcaagttcgagaaggccaagaagctggccgcagccaaacaccctacccatgccgaggagaccgctgcatgaccgcccagagttcttccactcaagccaggcacggcatgaccaccgacaacgaagtcctgctggaggtcaagggtctgtgcgtcgattacgtcaccgagtccggaaacatccgcgcctgtgacaacgccaacttcctccttcgacgcggcgaaatccttggcgtcgccggtgagtcggcgtgtggaaagtcgaccctgctcaacgcactcggacgcctgcagcggatgcctgccgccacgagcgctggccagatcctcttccatgatcgcaacggcagcgtcaccgacctggcgaccctgtccgaggcggacctcaagccgtatcgatggaccaagatcgccgtcgtcatgcagtcggcaatggcctgcctcaaccccatcctcaaactgtccgagcagttcatcgacgtccagcgcgcccatgatccctcgttggcagagaaggatgcgttgaccaagtccgccgagctcttggaggcggtcgggatctccgcagaccgactctcgtctttccccttccagctttcaggtggcatgcagcagcgtgccctcatcgcgctgtccttggtgtgcgagccggatctggttctcatggacgagcccaccacggccgtcgacgtcgtcatgcagcgacagatccttcaggaggtgctagcggcccagaagagactcggattctcgatcgtcttcgtcacccacgacctgtccctcctcatggagatctcggacaagatagcgatcatgtacgccggacggatcgtcgaggttggcaccccggcacgattccactccagtgcccgtcacccgtacacccgcggtctgcgtctggccttcccgccgctgagcgagccactgcgtcgactcgaaggcatcaagggttctccccctgaccttctcgacctgccaaccggttgtgccttcgctccgcggtgcgaatttgccatgccgatctgtcatcaacgaatccccgatcttcgcaccatcgacgagggagcgtctgcctgctacctgtcaaacggagagctttccgatcaagaactggcggaccagcagaagcaggccacgaatcatgaggaggcgatcgcatgaccggcgcccacgtcgacgaccgcaccgcggtccttgaggcacgcaacgtcagtctggatttcgacggcacggattcgtcaggatccaaggtgacgatccacgccctcgaccgcgtcaacctgaccattcaccgcggcgagatcactgccctcgtcggtgagtcgggttccggcaaaacgtcgatcgcccgtcttttcgccctgatctacaagcccacgagcggcgagctctaccgtaacggcgagttggtgcacgtgcacggcaagcgtgccgaacgngcctactatcgagatgtccagctcatctaccaggaccctttcgcatccctcaacgggctgaagaagatctcgacaatcctgggccgggtcttcaagatccactacccgaagatgcgtcgcaaggaaattgctcagcggattgacgacgtcttaacaaaggtcaacatgacaccgccgtcgcgctacctcaaccgctaccccactgacctttcgggtggtcagcgtcagcgcatcgccatcgcccgagctctggcggtcagcccgcaggttttgctggccgacgaaccaacatcgatgctcgacgcctccatccgtctggacgttctcaacctgctgtcggatctgcgtgagaccgaaggagtgtcggtcctctacatcacccatgacatcgcatccgcgcgctacatctgtgaccgcatcaacgtcatgtacggcggtcgcatcgtcgaagctggcccgactaagcagatcatcagcgacaccattcacccctatacccgcctcctcttgtccgccgcgcccgatccggctcggtacaagggatcggccaacagcaccgcgatcgatattcccgaaggcgcccccatgaataactccgtgagggttaaaggatgtcgtttcgcaccattatgtccgttggcacaaccccgttgcaccgaggaggagttgcctaagttccacagcgaggacggcactcgggaagtcacatgctgggaggcggaagaacggagggaattccatcgagtatgacaatacgtctggtatgttctctccatagaaggcagtcattgattttgtctccaaaaagacacattaatacccaaaggagggtattttaacatggacccgaaaccacttagttttccaatttcgcggcgatccttgctcttcaccgcagggtcgattgccctcaccattccgctgctgagcgcatgcggcagtggtgacactgacgccaagggcagcagaagcaagaagggcaccatcgccaaagacggcgtattgaccatcggcgccgcagtctcgacctcgaacgtcttcccccacaacttcaacgtgttcggcggtggtgattccgctcctggcaacgccatgttctgggagaccttgttccgcatctcctccaaggacggcatgaagctggtgccgaatctcgctaaggaggtcaaatacaccagcggcggcaaggtcgcaacgtacaagctgcgcgacgacgtcacctggaatgacggaaagccgttcacctcgaaggacgttgccttcacctatggcttcatcttcggcccacccggacaatccaccgagaaggatttcaagtggctcgctaagccgattgaaacccccgataagcacaccgttgtggttacctataatgagcctcagtataccgaggatattccgctctcgctgtacttcccgatttacccggagcacatttacagcaaggttgaccaccagaattatgtggacaagaatccggttggaaccggtcctggaaagctgaagtctttcgccggccagctcattgaaatcgacattcgtgacgattactggggcaagaagcccaagggcgtcaagaccatcaagctggtgccggcaggaaccgctggcaacattcagtcccagatcacccagggcaaggtcgactgggccgagggcggcgaccctggcgtcatcaccagcttcctcccgatggacaaggaccacaacggctacaactactacgccgacggttcgacccgaggaatcatcctatcgacccactccctgccgacctccgacgtggccgttcgcaaggccctgcgagctagcgtggacatgggcgtcgtcgccaaggctggcggcgtcggatacaccgtgcccagcgtgaccggcctggatcctgtcatctacgccaacatgctgaagccggagttcaagaagccgatggctccggatgtcgaggctgccaagaagttcctcaaggatgccggctggaccgttcagaatggcaacctctgcaaggacggcaaggaatacccgctccagctgaccattcgtaatgacaacccggtcgagatggccactatgccgatcgtcgtctcccagtggaaggacaacctgggcatcaacgtcaagttcaccccgctgcccaaggaggtcttcgacccggcccaggccatgggcgagtatgacatgtcgctgtggacgaccaacgccgccggtggcgcgttcaacgcctacacgatgtacatgcagacgaagaactacaaacttggcgacaagaaggccgacggcaactacggtcgctggaagtgctccaaggaagccgacgaagccatcgcgaacctccagaaagtgccgcaggacgacatcaaggagatcactcgtcgttgccagatcctgcaacaggccgtctacgacgatgccccgtacattccggtgcagaactcgggtactggcggcatgtacaccaccaagaagtggagcggcctcaagaaggcggaggacgtcgactactttccgcgcgccagtggctacaacaatatgattcacacggtcaacgacttcgactgatgtaatcatgaatgatgcctggggccttacggagccccgggcatcgtcccactctccccacaaccagaggttctgatgccaaccgatcaccggatcgtggcctaccaccagacccatgggcacggcgacaccttcgtcgatctgtcccccctgggagcgagcgccccactcacccacctcatcgtcgcggccttgcacattgccgacgacggcaccgtcattctcaatgatcatccggttgacgacgactaccacacccagttgtgggaccagctcgatgcggtgagatcgcgcggtgtccgcgtcctcgccatggtcggcggctgggccccgggcacgatgtgcaaactcgacggcgaatcattgtcgatctactacccagggctacgggatttcctgacgcaacgccatttcgacggcatcgacatcgacgtcgagcaagagatgtccttgcccggtgtgtgcaccctcatcgaaaggtttcgcttagacttcgacgaggacttcgagatcgttcttgctcctgttgcctcggcccttcatggtggtgccaacctgtctggctttgactatcaagaactccaccagctgcgtggggacgacatcgacttctacaacgcgcagttctactcaggtttcggctcccttgcgagccctgacgactacgtctccattgtcgagcacagcatcatcccggtgaataagatcgtcactgggatggccggcagtccagacgccgcacagggtttcgtcgacatagccactgtgtcagacaccatttccacgctgtgccaacgttacccaaatttcggcggtgtcgacgtctgggaataccccttggcgttaccaggagcgccggaccatcctgaccaatgggttcatgtcatgcatcacgcgatgtctcaccgctgacacctaccaccaccccctatttcacacgacgtgtccatgaccattcatcc

SEQ ID NO: 11 - DNA, 761 bp, Propionibacterium acnes
accaactcgacgaaccactcgagttcagcgccaactgggataccggtcaaggccgctcaacgtgctccccgataggcgatctcttcgctcagtgggttgaccaggcagaacgaaccgggaaggctccgcggcttcgccgagtgcgaacgcaacattgccctgtgccctgagcgatgggcgcttattagcatcgttgtatgcgtattttagttgtagcagcccgtggaacaaatggaaaaggtaaccaccttgtttcgcaatcatgaccgtacgatgattgaagtagacaggtctgagggtgagattctgtgcgacatcagcaatgaaatacatctggattcattgtggcggcaggcggtccatattgacgccgtagcgtgcgcatcggggacgacacccttcgcacccttgaaggagttgaccttagccaatttcaacgatgactgacaaaacaaggcgctttcccaaataaacctcgtgcgtacaggcattaatcatatttccgagcggggatccttcaccttgacgagcggaatcctctcccgtgatcctattcccaagggatcggcaaccgatgtcgccaacgctttccgaaaatcataactcccaaagaggaatggtgcgagcagagttctccaatatctgaagaaatcattaatgcgggaatctttcatgcaacgcggtattaaccattcccggcacaaactcggatacattgccgccattacaagcagcagagcgaatcatcgaggagctgatcg

SEQ ID NO: 12 - DNA, 20 bp, Propionibacterium acnes
accaactcgacgaaccactc

SEQ ID NO: 13 - DNA, 20 bp, Propionibacterium acnes
cgatcagctcctcgatgatt

SEQ ID NO: 14 - DNA, 20 bp, Propionibacterium acnes
tcgctctgctgcttgtaatg

SEQ ID NO: 15 - DNA, 901 bp, Propionibacterium acnes
gtgtccgatcaccgatacctggtggcagaccgaaactggcatgttccagatcaccactgtgccgtcgatgccgctcaaaccaggtgccgttgggcgtccggtctttggccaggaggctgctgttgtcgacgaggagggccatgagctccctgcgggcaaagagggcttcctcgtcctcaaaaacccgtggccggccatgatgcgcaccctgtataaggatccggatcgatacctcgaggcgtactggacgaagtacccgggtgtctatctcaccggtgattccgcacgaatcgatgatgacggctacatatggatcatcggacgtgctgacgacgtcatcaaggtatccggtcaccgcatcggtactgctgaggtggagtccgctctgatctttcaccccgccgtcgccgaggctgcggccatagggctgcctcacgaagtgaagcgcaacgcgatccatatggtcgttgtactcaatcatagtttcgagccgacgaagaatctcgtctctgatatccgcggacacgtcgccgaaaccctctcgccgattgccaagcctgacaccatcgagttcgtcgacaagttgccgaagacccgctccggcaagatcgtgcgtcgcgtcctcaaagctcgtgtcctgggtggagacgagggagacctgtcaaccctagaggactgaggccattaccgggaggggcggggtatgccatgtcctcgtccctcgtgcttgtcgaccatgtcagcggtgtccttgccaggcccgcattgactgtgagtacgtgtcggcgagatcaggctgcgacagaatatgcgtgccgtaggagttggcaaaattgacctgtttatctgttcggtgtgatctacagccattgggcaaaattctttgcagcctcaacttcgt

SEQ ID NO: 16 - DNA, 295 bp, Propionibacterium acnes
gtgtccgatcaccgatacctggtggcagaccgaaactggcatgttccagatcaccactgtgccgtcgatgccgctcaaaccaggtgccgctgggcgtccggttgctgttgtcgacgaggagggcaatgaggtccctgcgggcaaagagggcttcctcgtccccaagtgagcgcgtgtcggcgagatcaggctgcgacagaatatgtgtgccgtaggagttggcaaaattgacctgtttatctgtttggtgtgatctacagctattgggcaaaattctttgcagcctcaacttcgt

SEQ ID NO: 17 - DNA, 296 bp, Propionibacterium acnes
gtgtccgattcaccgatacctggtggcagaccgaaactggcatgttccagatcaccactgtgccgtcgatgccgctcaaaccaggtgccgctgggcgtccggttgctgttgtcgacgaggagggcaatgaggtccctgcgggcaaagagggcttcctcgtccccaagtgagcgcgtgtcggcgagatcaggctgcgacagaatatgtgtgccgtaggagttggcaaaattgacctgtttatctgtttggtgtgatctacagctattgggcaaaattctttgcagctccaacttcgt

SEQ ID NO: 18 - DNA, 20 bp, Propionibacterium acnes
gtgtccgatcaccgatacct

SEQ ID NO: 19 - DNA, 20 bp, Propionibacterium acnes
acgaagttgaggctgcaaag

SEQ ID NO: 20 - DNA, 443 bp, Propionibacterium acnes
  misc_feature 342: n = A,T,C or G
gccatcctgttcatgatcgccggtgtcatgcagaacgcgggctggctgctggtgtccctcgtcattggctgcttcgaatggtccggcggtgcccgtcagatccgcgcccaggcaatgtccctgcgaggacgcgatttcaccacggccctgcggaccatcggtgaatcgcagtcccacatcgtgctctcagaggtcatgccgcacctgctcggcctcatctccccgatgttcctgcgactcatcgccgccggcgtcaacatgcaggcgtccctagccttcctcggtatcggtgacccgtccatgccgtcctggggcctcatgatcaactgggccatgacgcanaacgcgctgttccgcggtctgtggtggtggttcattcctccgggtctcgcactcgcccttatcggtttcgcaaccaccatgatcaacttcggtctcgacga

SEQ ID NO: 21 - DNA, 19 bp, Propionibacterium acnes
gccatcctgttcatgatcg

SEQ ID NO: 22 - DNA, 20 bp, Propionibacterium acnes
tcgtcgagaccgaagttgat

SEQ ID NO: 23 - DNA, 584 bp, Propionibacterium acnes
gacgattactggggcaagaagcccaagggcgtcaagaccatcaagctggtgccggcaggaaccgctggcaacattcagtcccagatcacccagggcaaggtcgactgggccgagggcggcgaccctggcgtcatcaccagcttcctcccgatggacaaggaccacaacggctacaactactacgccgacggttcgacccgaggaatcatcctatcgacccactccctgccgacctccgacgtggccgttcgcaaggccctgcgagctagcgtggacatgggcgtcgtcgccaaggctggcggcgtcggatacaccgtgcccagcgtgaccggcctggatcctgtcatctacgccaacatgctgaagccggagttcaagaagccgatggctccggatgtcgaggctgccaagaagttcctcaaggatgccggctggaccgttcagaatggcaacctctgcaaggacggcaaggaatacccgctccagctgaccattcgtaatgacaacccggtcgagatggccactatgccgatcgtcgtctcccagtggaaggacaacctgggcatcaacgtcaagttcacccc

SEQ ID NO: 24 - DNA, 20 bp, Propionibacterium acnes
gacgattactggggcaagaa

SEQ ID NO: 25 - DNA, 20 bp, Propionibacterium acnes
ggggtgaacttgacgttgat

SEQ ID NO: 26 - DNA, 443 bp, Propionibacterium acnes
gtccttagcgatgaggacagtagagcgggatgccatgctggccattctcccgcacttgcaagacccacctcaaccggcattgactcatggaccaacctgcccgagggggtcaaggggacacgcaaggctacaagaccacccaacccgcccgagggggtcaagggggagtcgcctccccccccttacaataaaacccgtgttgacaacccgagccaccctggacaaacaccacactgacgtcgcatcgatgttcgacggggtggcgaaacgctatgacctcatgaaccagatcatgacgctcggggcgatcgacacttggcgcgacctcgtcgtcgctgcggtagagccggaacccggccagaccatcctcgatttggcggctggaaccggcacctcctcagctacgtttgccgcccgcggcgcgcaggtttatcccaccgata

SEQ ID NO: 27 - DNA, 545 bp, Propionibacterium acnes
gtccttagcgacgaggacagtagagcgggatgccatgctggccattctcccgcacttgcaagacccacctcaaccggcattgactcatggaccgaccagatcggggggatcaggcacgacacgcgagcccctcacccccacagagactcaacccgctcgagggggccaacaggacacgcaagattacaagaccaccccacccgctcgaaggggccaagggggggggagtcccccgccgcacaaactcaacccgctcgagggggtcaagggggagtcgtctcccccttacaataaaaccgtgttgacaacccgagccaccctggacaagcaccacgctgacgtcgcatcgatgttcgacggggtagcgaaacgctatgacctcatgaaccagattatgacgctcggggcggtcgatacctggcgcgacctcgtcgtcgacgcagtagagcccgagcccggccagaccatcctcgatttggcggctggaaccggcacctcttcagctacctttgccgcccgcggtgcgcaggtttatcccaccgata

SEQ ID NO: 28 - DNA, 440 bp, Propionibacterium acnes
gtccttagcgatgaggacagtagagcgggatgccatgctggccattctcccgcacttgcaagacccacctcaaccggcattgactcatggaccaacctgcccgagggggtcaaggggacacgcaaggctacaagaccacccaacccgcccgagggggtcaagggggagtcgcctcccccttacaataaaacccgtgttgacaacccgagccaccctggacaaacaccacactgacgtcgcatcgatgttcgacggggtggcgaaacgctatgacctcatgaaccagatcatgacgctcggggcgatcgacacttggcgcgacctcgtcgtcgctgcggtagagccggaacccggccagaccatcctcgatttggcggctggaaccggcacctcctcagctacgtttgccgcccgcggcgcgcaggtttatcccaccgata

SEQ ID NO: 29 - DNA, 20 bp, Propionibacterium acnes
gtccttagcgacgaggacag

SEQ ID NO: 30 - DNA, 20 bp, Propionibacterium acnes
tatcggtgggataaacctgc

SEQ ID NO: 31 - DNA, 508 bp, Propionibacterium acnes
gcgaccggtttgttgtgagctgtgttgctgacgattccagcttcttcgtactctttgaccatggctgcgatagcggcggatgcattcgcgaagaccatgcgcgatacgtgattgatgtcggccacgttaacgacggagtacataagtgtgacgtcggcctgtcccacgtaggctctggcatctccggaagacatcgtggaaaccaggagcttggggaatccaatcggctatgcggctgttggagaatgagccaacatcgatgatgactctaccttgctgccgttggtaagaatgcggtgacgaagccagtcgagctcgtctccctcggtatccaaggttccgaacacagcaattgcggccatgaaacactccttcgtgatcatgtcatcatgaccgtagttcggtgattctgcctgcaccatgctgcttgagacgaattggcctatatggttgtaccgctggttgcgaagaagggcaatactccaggtgtggttgtcgaagttggccc

SEQ ID NO: 32 - DNA, 716 bp, Propionibacterium acnes
acgaccggtttgttgtgagcggggttgctgacggttctaacgtcttcatactccttggccatggctgtgatagcggcggctgcattggcgaagaccatgagcgacacggaattgatgccggccacgtcaacgacggagtacataagtgtgacgtcggtttgtcccacgtaggggctgacatctccggaagccatcgtggaaaccaggagcttggggaatccaatcggcaatgtctgcatgaccgaagccgctacggatgatccacctgatccgccgagggacaggagaccgtggattttcccctcttcggcaagcttggatgcaataatcgaagcgccacggcccatgacatccatcatttcgccacggttatgacgtgttcggatgtcacttggcgactcgcctgcggcacgaatgacctcatcggacgtgtagtcggctatacggctgttggagaatgagccaacatcgatgatgactacctggctgccgttggtaagaatgcggtgacgaagccagtcgaactcgtctcccttggtatccaaagttccgagcaaagctattgcggccatgaacactccttcgtgatcgtgtcatcatgaccgtagttcggtgattctgcctacaccatgccgtttgagacgaattggcctgtacgttgtatcgctggttgtgaagaagggcaatacttcaggtgtggttgtcaaggttgaccc

SEQ ID NO: 33 - DNA, 19 bp, Propionibacterium acnes
acgaccggtttgttgtgag

SEQ ID NO: 34 - DNA, 20 bp, Propionibacterium acnes
gggtcaaccttgacaaccac

SEQ ID NO: 35 - DNA, 2279 bp, Propionibacterium acnes
catcccgagcagctcggtcagccccagggcattgcgcagcacgtcgtcctgaccgacattgccagctgtcgtcaccaccccgacggtctcgacatcgtcgcggctcgccaggtaggccagcgcgaacgcgtcgtcaatcccagggtcgcaatcgagcagaaccggcaagaaacgtgtgggcatggcgtcagaatagaggccctccaccgcaccaccgatcgttgagtcgtcccacctcaccttctgctgaggctgttcgggctggtcagagcgcctttgctccaggactcgcacatccgcgtgtcctgccccctcattgtccaacgggtatgtgccacacatttcactgtcccctggtgccgccctgcccgaatcttggaaggaatcatgctcgactgggtggacctcatcattcgtatcggagtcggcgtcggccccggaaccctcatcgagataggagccgacggattcggtgcaactgatcccccataccctccatacggacacgtctcgtagcatccccagtccggcctggagcccctcggtttggtcgtgagccttcctggaggacacttggccttgtcagagcaggtcgtcagccacgtgcgtgacgacgctggcgagcgagtgtgaggaagtcatgaccaygaggagaaggaaccgtcccgcgctgagaggatccttgcgggcaccgtcgcgtttacgatctgcatggccgtggtgttcgtgatcgtggcgatcttcatcggttggcgctggtcgctgaggatgttcgtcggagtcctgctcatcgaatccccggcgagatcctgctggaactggttggcgagaaccgaatgttcccccgcggcggaggcaaaccgggccagacacgagctgcacgtttgctcatcctgacgctcatcctcgccgcgtgggctctcatcgtcctcgccctcaccgccctcatccaaccgtggtacggcgccgtcggggaaatagcgtgggtcatggtgatcgggatcctcgcgatcctcatccaggggatccgggacacccggaagaagaagtcctagcccctcgccatgatcacagcgccgagactcctccctggcggccttttgtacctctcacgccaggtgctcaggacgtcggagaccgtcggctgcttgtccgcgggaacgccggactggaactcgtcgtcgtggatctccatgtcgaagtgggccgacacagcacgtactctcctctagagaggcctagaccccttggaattgtcgaacatgtcgtcaaggtgggtgggcaggatagtctggccacactggctggatccatcgagattgtggggcaatggctgccttgcagataccaatccgatcggcgcgaccggtcactggcccacacccgcagcacgaatctcaccaggaaaggaagcgttgaccatgaagatcaacgacaacggccccaaaccgaacgccttcgacatcgagaccgcaacgaaggagaacacgaactaccgcaccaccgcctggaccggtaagtacctgcaggtgacgctcatgtccatcccggtgggagagtccatcggcctggaggcccaccccgacaccgatcagttcctgcgcctggactccggcaaggggcgctgcgtcatgggaccggctgaggatcagctcgacttccagcaggacgtcagcgacggctggtccgcccaggtcccggccggcacctggcacgacgtcatcaacaccggtgacgagccgatgcaggtctacgccatctacgccccggtccaccacaccccgggcatcgtccaggagaccgctgccaaggctgaggaggacgaaaagtccggagccgacgtcccaccggagtggagcgtccaacccgacaagaccgctgacgatctgcacgcctgaggaataccatccgtcagagaccaggccgtcgccaacgagggcgacggcctggtctctgacggaacggcgtcatcaccggtgtcaccgccccgacacatggatctccctcatcgaccgcgcgatcctcggattcgccggggcctgcctggccatggcgtcgatcggtgccgtcactgtcgtactgaccggccgagaccgtcagctcaccctggccttcaactccgcaagctgggccgtcgtcgccctcgacttgtggaggcgggatcgcgggatggggatgctgcaggaggattcgcggtctgggaatgcgatcacttctgtgcgaccgagccgggaaacagcccgagctccatgccggcgatgatgaactcaaccttgaga

SEQ ID NO: 36 - DNA, 322 bp, Propionibacterium acnes
cattccgagcagatcagtcaccccaagcgcgtttcgcagtacgtcgtcctgccccacgttgccggcagtcgtcaccaccccgacggtctcaacgtcgtcacggcaggccacgtaggccagggcgaaggcgtcgtcgatacccggatcgcagtccaacaagacgggcagggagcgcgtagaagtcgacatgtcgccacactaaggcttcgaccgttgtggcgtcaccctccgatgcgtccaccaaacttgtagcgggattcatgaagaagcgccgcggaacagaccgtgctccatccccgcgatgatgaactcaattttgaga

SEQ ID NO: 37 - DNA, 20 bp, Propionibacterium acnes
cattccgagcagatcagtca

SEQ ID NO: 38 - DNA, 24 bp, Propionibacterium acnes
tctcaaaattgagttcatcatcgc

SEQ ID NO: 39 - DNA, 725 bp, Propionibacterium acnes
gtcatcggcttctggatcatagaaatcttcacctggggtatctattcctgcgacccccagagtaggaatgaaagcttgagccgggccgcgcgcagcggccgcctttatggcgattctgtgaccaagctcggccatttcggtgttgctcgtttgagcgtcaccgctggattgtggatatgcaacgtgcgagaggaatagcgctccggaatagtgtcgacagggccgaaattgaccatgtcaagcgcgccaagactgatgacttcgggtattccaaggtctgctgcggcttccatccggcggggtcctgccgagagaactcctccgcagaggtcgtcggcaagctcggtagtcgtcaggtcacatattcccgcgagtagcccagacgatgcgagcttttctatcgccttcccctcccggaatcagttgcgtggaagacgaggacctcgtacccaaggtctgtcaaggtcttgcgagcttcgtcagcagccggcgttgtcaatccgaacatcgtcaggccgacgaccggtttgttgtgagcggggttgctgacggttctaacgtcttcatactccttggccatggctgtgatagcggcggctgcattggcgaagaccatgagcgacacggaattgatgccggccacgtcaacgacggagtacataagtgtgacgtcggtttgtcccacgtaggggctgacatctccggaagacatcgtggaaaccagg

SEQ ID NO: 40 - DNA, 20 bp, Propionibacterium acnes
gtcatcggcttctggatcat

SEQ ID NO: 41 - DNA, 20 bp, Propionibacterium acnes
cctggtttccacgatgtctt

SEQ ID NO: 42 - DNA, 618 bp, Propionibacterium acnes
gtacgaggcttccgtagcactccctccgcgagaattcgtgaatgtaagggactatcaagcccacttcttgagcgcaaatcaacaacttcaaacgaaatttaagtcatggggaaaaagggaaaaattccctttgcctgacattaatatttcagacctacatatacagattgacgaccttctttttttagctgctgcccttaggtttgggttcggaatagacccggagcggaccacttcaaaaatagcgaagcgctggacggcctctggtggaaacattggctcggtaaacgcattcgtgagccttcctgcgatggtagggcacattcccagtgggatttatggatattcagttagcgatcattccctagccaaagttagtcaggagttgatttcggcgactgacatcatgatcgcggcttctgccgacctacggaagatcgcttctaaatatgggacattcggcttgaggatcgcgattatggacgccgggtgtgccctttcaacggtgcgtcgtgtctgccgggaagtaggccgtacattttcgatgtggagcgctgacctcgacgctgggtctattagcgagatgctgcatctttcaagtgcgcgtgagcctattat

SEQ ID NO: 43 - DNA, 20 bp, Propionibacterium acnes
gtacgaggcttccgtagcac

SEQ ID NO: 44 - DNA, 20 bp, Propionibacterium acnes
ataataggctcacgcgcact

SEQ ID NO: 45 - DNA, 4027 bp, Propionibacterium acnes
ataaacccatcggcggctcgatccatcaaggccgtcggcggacgtgagcgagccggaatcgtcatacccgtaccggaaatccccgtatggtgcgctcacccggagtactcggcccatctcgtcgcgactgatggtcgtggacccgcgggaggaggtgtggctagtgacgaaaccatcagtatacccccactgctgttgatccagaggactacctagcttcgcgagacgaccgaccgcgtcccattccaagctcacctcacccaatcgagagtggcgaatcgaccccaatacaccggtgttggtgtatgcgtaatcgatagaatcaccagtcaacgtgagatgacgtacacgacgcccctcggcgtcgtaccaccattgttcggacgaggtgagtggcccctcgtccacaatcgattcgtctcccggcacgggggcgacgctgtgggtcaagagcagcccacgagggtcgtaccccaacaggtggcgccacgggaaagcaggatcggtgtagtcatcaatagtgaccgtgcgggtggtcggcgtgatgaggatacgactgaccagctggtcatcaatgtattcctcgacgcctttagggccaagacgacggctgacaaccgtgccatccgggcgggtgatggcggccagggttccggacttgtcccaggtatattcggtgactcgccccagtgggtcggtggcggcgatcatctgcccgcactcgttgtactgatacgtggtgaggccaccacccggatcggtgcgggtgacgagtcggccacaccgatcgtagccgtaccgtgagacgccaccagccgggttagtcgctgccaccagttggccggcaaggtcgtaactgaagtgacgctgtccgtacactagatcgcaactgaacacgagtctgccgcatgggtcatatcggtactggctcactccagcgccaggaacatgactgagcacaacacgtcccatggcgtcatactcgtagcgcgcggtctcgccagttggataagttactgtcgccacctggaaatctgaggtgtaggaatagcgcgttacgcggcctgcgggatcggtgacatcaaccatcctcccacacgcgtcatagctgtaactcgtgcgcagcccggtggggctgacgagggccacaatgcggcccgctagatcgcgctccacatgagtcaccgcgccagactcgtcctgaaactcaaccggtcggccacacaaatcacgcgtgacgatctgctgggcgccagtgacatcggtgacggcagtagtaagaccgtagggatcggcatcgacgcggtggagttggtgcccgtgactatccgaggcggtgctggcagtgagaccgtcagtaaagaccctggcctggcgaaaccggctggatcagtgacgccggaaagattgccatcaacgtcataggcatacgtccacctggcctcgtccggtgccaccacctgcgtcaaccgcgatagcgcgtcatggatgaagctccaagcgctgccgtcggggagctgcatgcccgccaggttccccaagtggtcgtaggagtgttcaatgacgcgcccgagaggatcgatgaccctggagatctggccgtctgggtgatactcccactttgtgaccccactatcgggggcaaccaactcgatcagatgaccggccacatcataacgatgctcccatgtcgcgccatccggtgtgaccacggcagccaggcggccagcgtcgtcgtagctgaaccgcgtcgtggcgccacccggcgagatcgtttcgacgatccgtcccgcctcgtcacggatgaggcgggaagcctccccatgggcatcctcgacgcgcaccaattctgcatgatggtcataggaaaacctcaggctcaccccgacaggattcgtggcgcgcagaagcaatccatccttccactccgcaacggtgacgccaccgcacggatccgtgacgcgcactggatcacgctcggtgccgtcatactcgaaggtggtctgggcgccattggccagggttgtgctcacaagccggtccagatcatcccacgaacagtcaatcgtgcctccagtaggaaggtccgtgtgcgtcagtctccctcgctggtcatagcggtgtgaggtaacgtttccagcccgatcgacgcaacgcaccatattgccgaaggaatcgtaggtcatcgaaacctggccaccatgggcgtcgaccacacctgtggtgcgaccgtgaggatcacatatccaggtattggcattggtgccatccgcatcgctggcggcggtgacgcccccaggcagatacctgaaatggacagtacgaccgttagcggcttgttgggacgtgatccggccccggccgtcataactgttgatgcattccgcgttgccacatgcatccaccacggtcgtgatgagtgtgtcatcccactcgtaacgccgtgaaccgtcaggcccgtctacctggacgagtcgattttcgtcatcataggagtaccggacacaacggccatcactggaaatagccgacaccactctggcgccgccatacgacacggtgatcttgtgaccccacgacgtttccatcgcggtcacgcgatcgccttcccgcacggtatgcacgacgtctctttgcgaagatccgctgcacacccatgcgccaccttcagtgaaaacccatcgacctccccggttgtccgagatcacccacacccattttgatgtggccggatcgcgaaggaactccggaagttgggaagcagagacccggctgagccacagattctcaccctgagctcgccactgttcgacaggcttttcgtcttcctctgccggattgggcagctgattggtcgtcggcaggactgctgtcggtgcagcctccaccgcgaaggcgatatgcctgccatcctcacgcacccactcgacgcaccccggcttgacgagcaggcactggtcaaggatgctgacccaccccgggccgaaaaccccaccctgtccccgaacagcctgaatagagttgtacatcctgctgagggcgaggggggatgacgctgccgcgaaggccagatcggtctcgggctcgatgaaattcccggttgccgagttgatgggatcctcaacgtatccagtgcgcggatcaatacccgacagccccggggatgtgatgtcgagatcggtacgccacaaaggggttccagcagctctcaaggcggctcgcagggcagagttaggcaaggtaataacaccagagccagcagcttgaaaggccttggctaccgcgtgcaaccactcgacgtctccgtcattgaggccccgccaccgatccagctgcacgaacagatccccgacctccacggtgccgtacttgcactgggaggtgaagtcgtcgaaccagcccgccaggactgatcccgagggtacctgtgctgcggtttcccccaacacctgcgcggcactgatgagatccttggggtccgccgcagatattccacccgatgagcctcggatcgaccgatcctgctgacgatctcctgtcacaggcggatggagcagctggggggagggaggctcggcaggagtgaggttgggcggaacatcgacgccggtgaagaaatcgcgcacatggtcaccaaaatcgtcatgttgcgcagcgaaatcgcgaacctgttggcgtcgcttattctctaaccgagcttgctcggcaagatagtccaccatgtcggcggcggtggtcaatgcctctgacaacgccgtagcgtccgcggaacatatgtggatgttctgtgtgaagacagtcgcgaagaatccccgaaaccctcgtgaggcgatggactcaacgagcgacgttcgcgcgtcaaaaatgtttatgcgttgcgaggtccaacgcagctggctagagaacccttcggctgttgagaaattgaattctacgggattctggtagtcgccgaaaacactgaaatcgaccactgaatgctcccttgctctttgggcaaagtgcagcgtgatgctccactgcctgg

SEQ ID NO: 46 - DNA, 573 bp, Propionibacterium acnes
  misc_feature 28, 32: n = A,T,C or G
ataaacccatcggcggctcgatccatcnaggncgtcggcggacgtgagcgagctggaatcgaccgatcctgctgacgatctcctgtcacaggcggatggagcagctggggggagggagactcggcaggagtgaggttgggcggaacatcgacgccggtgaagaaatcgcgcacatggtcaccaaaatcgtcatgttgcgcagcgaaatcgcgaacctgttggcgtcgcttattctctaaccgagcttgctcggcaagatagtccaccatgtcggcggcggtggtcaatgcctctgacaacgccatagcgtccgcggaacacacgtggatgttctgtgtgaagacagtcgcgaagaatccccgaaactctcgtgaggcgatggactcaacaagcgacgttcgcgcgtcaaaatgtttatgcgttgtgaggtccaacgcagctggctagagaacccctcggctgttgagaaattgaattctacgggattctggtagtcgccgaaaacactgaaatcgaccactgaatgctcccttgctctttgggcaaagtgcagcgtgatgctccactgtctgg

SEQ ID NO: 47 - DNA, 22 bp, Propionibacterium acnes
ataaacccatcggcggctcgat

SEQ ID NO: 48 - DNA, 20 bp, Propionibacterium acnes
agcaagggagcattcagtgg

SEQ ID NO: 49 - DNA, 23 bp, Unknown
  Bacteria universal 16S rRNA
tgaagagtttgatcctggctcag

SEQ ID NO: 50 - DNA, 24 bp, Unknown
  Bacteria universal 16S rRNA
ggactaccagggtatctaakcctg

SEQ ID NO: 51 - DNA, 19 bp, Unknown
  Bacteria universal 16S rRNA
gtgccagcagccgcggtra

SEQ ID NO: 52 - DNA, 20 bp, Unknown
  Bacteria universal 16S rRNA
agscccgggaacgtattcac